(19) United States
(12) Patent Application Publication
(10) Pub. No.: US 2016/0296506 A1
(43) Pub. Date: Oct. 13, 2016

(54) Methods and Compositions for Treating HIV Infection

(71) Applicant: Vanderbilt University, Nashville, TN (US)

(72) Inventors: H. Alex Brown, Franklin, TN (US); Craig W. Lindsley, Brentwood, TN (US)

(21) Appl. No.: 15/189,920

(22) Filed: Jun. 22, 2016

Related U.S. Application Data
(63) Continuation of application No. 14/103,795, filed on Dec. 11, 2013.
(60) Provisional application No. 61/735,998, filed on Dec. 11, 2012.

(51) Int. Cl. A61K 31/454; A61K 31/435; A61K 9/00

(52) U.S. Cl. CPC A61K 31/454; A61K 9/0053; A61K 9/0019; A61K 31/435

(57) ABSTRACT

Disclosed are methods of treating HIV infections comprising, in one aspect, administering compounds that are phospholipase D inhibitors. This abstract is intended as a scanning tool for purposes of searching in the particular art and is not intended to be limiting of the present invention.

### CROSS-REFERENCE TO RELATED APPLICATIONS

This application is a continuation of U.S. application Ser. No. 14/103,795, filed on Dec. 11, 2013, which claims the benefit of U.S. Provisional Application No. 61/735,998, filed on Dec. 11, 2012, both of which applications are incorporated herein by reference in its entirety.

### STATEMENT REGARDING FEDERALLY SPONSORED RESEARCH

This invention was made with government support under grant numbers U54 MH084659, P01 ESO013125, NIAID HHSN2722008000058C awarded by the National Institutes of Health (NIH). The United States government has certain rights in the invention.

### BACKGROUND

Viruses are minute microorganisms having no cell structure, and they are broadly classified as DNA viruses or RNA viruses. In some sense, viruses are not living organisms in their own right since they completely depend upon host cells for all aspects that characterize living cells. For example, viruses require host cells for protein synthesis and energy production mechanisms, and viruses completely lack their own metabolic pathways. In short, viruses cannot exist without the cellular machinery of a host cell. Thus, viral infection presents a particularly difficult therapeutic challenge, in part due to the significant difficulty of designing therapeutic agents that attack the viruses without significant collateral damage to the host cells and other cells in the body.

Examples of a RNA virus causing a human disease include Japanese encephalitis virus, hepatitis C virus (HCV), and the like of the family Flaviviridae, Rotavirus and the like of the family Reoviridae, mumps virus, measles virus, and the like of the family Paramyxoviridae, influenza virus and the like of the family Orthomyxoviridae, and HIV and the like of the family Retroviridae. There exist three modes of viral infection: acute infection with significant disintegration of host cells; persistent infection with clinical symptoms that remain at relatively minor levels but become chronic; and latent infection with viruses that remain in a state in which no observable viral protein synthesis takes place for a long time period, although cancer is induced in some cases.

As noted above, HIV is caused by a RNA virus of the Retroviridae family. There are two types of these viruses: HIV-1 and HIV-2. HIV-1 is the virus initially discovered. It is more virulent and more infective, making it the cause of the majority of HIV infections worldwide. HIV-2 has a relatively poor capacity for transmission and is largely confined to West Africa.

The total number of people living with HIV in North America and Western and Central Europe continues to grow, reaching an estimated 2.3 million people in 2009 (a 30% increase from 2001). The adult prevalence rate for North America alone was 0.5%. During 2001, 70,000 adults and children in the region became newly infected with HIV (UNAIDS AIDS Epidemic Update 2010).

Significant progress has been made in the last several years towards the development of anti-retroviral therapy to fight HIV, primarily targeting viral replication by interfering with the reverse transcription process and maturation of the virus. To date, a number of approved drugs have been shown to greatly increase patient survival. Indeed, the number of annual AIDS-related deaths worldwide is steadily decreasing (UNAIDS AIDS Epidemic Update 2010).

Despite these advances, new classes of drugs are required to overcome problems of drug tolerability and toxic effects, latent viral reservoirs, and drug resistance. Therefore, there remains a need for methods and compositions that are both potent, efficacious, and selective therapeutic agents for the treatment of HIV.

### SUMMARY

In accordance with the purpose(s) of the invention, as embodied and broadly described herein, the invention, in one aspect, relates to anti-HIV therapies. For example, compounds having phospholipase D activity (e.g., isoform selective Phospholipase D inhibitors) can be useful in anti-HIV therapies.

Disclosed are methods for treating a subject for HIV infection, the method comprising the step of administering to the subject an effective amount of a compound having a structure represented by a formula:

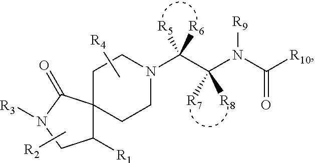

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

Also disclosed are methods for treating a subject for HIV infection, the method comprising the step of administering to the subject an effective amount of a compound having a structure represented by a formula:

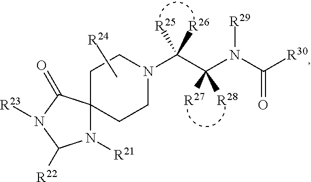

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

Also disclosed are methods for treating a subject for HIV infection, the method comprising the step of administering to the subject an effective amount of a compound having a structure represented by a formula:

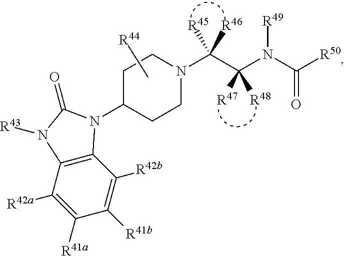

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

Also disclosed are methods for treating a subject for HIV infection, the method comprising the step of administering to the subject an effective amount of a compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof, thereby treating the subject for HIV infection.

Also disclosed are methods for treating a subject for HIV infection, the method comprising the step of administering to the subject an effective amount of a compound selected from:

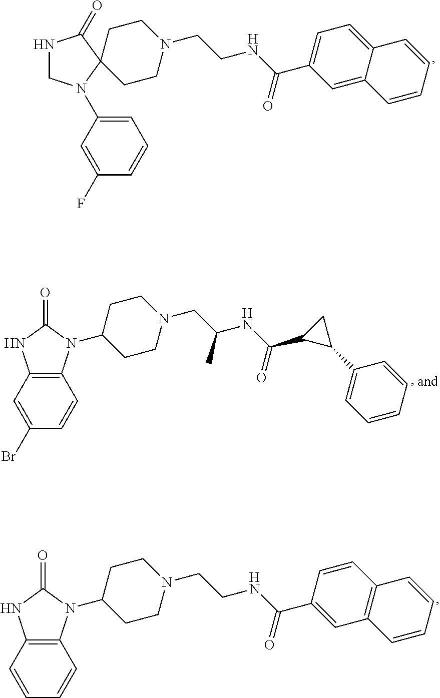

or a pharmaceutically acceptable salt, hydrate, solvate, or polymorph thereof, thereby treating the subject for HIV infection.

Also disclosed are methods for treating a subject for HIV infection, the method comprising the step of administering to the subject an effective amount of a phospholipase D (PLD) inhibitor, thereby treating the subject for HIV infection.

Also disclosed are methods for inhibiting HIV replication within a cell, the method comprising the step of contacting the cell with an effective amount of a phospholipase D (PLD) inhibitor, thereby inhibiting HIV replication within the cell.

Also disclosed are methods for inhibiting HIV integration within a cell, the method comprising the step of contacting the cell with an effective amount of a phospholipase D (PLD) inhibitor, thereby inhibiting HIV integration within the cell.

Also disclosed are methods for treating a subject for HIV infection, the method comprising the step of administering to the subject an effective amount of a binding agent of phospholipase D (PLD), wherein the binding agent binds to at least one amino acid in a non-catalytic domain of PLD, thereby inhibiting HIV replication within the cell.

Also disclosed are methods for treating a subject for HIV infection, the method comprising the step of administering to the subject an effective amount of a binding agent of phospholipase D (PLD), wherein the binding agent binds to at least one amino acid in a catalytic domain of PLD, thereby inhibiting HIV replication within the cell.

Also disclosed are methods for inhibiting HIV replication within a cell, the method comprising the step of contacting the cell an effective amount of a binding agent of phospholipase D (PLD), wherein the binding agent binds to at least one amino acid in a non-catalytic domain of PLD, thereby inhibiting HIV replication within the cell.

Also disclosed are methods for inhibiting HIV replication within a cell, the method comprising the step of contacting the cell an effective amount of a binding agent of phospholipase D (PLD), wherein the binding agent binds to at least one amino acid in a catalytic domain of PLD, thereby inhibiting HIV replication within the cell.

Also disclosed are methods for inhibiting HIV integration within a cell, the method comprising the step of contacting the cell an effective amount of a binding agent of phospholipase D (PLD), wherein the binding agent binds to at least one amino acid in a non-catalytic domain of PLD, thereby inhibiting HIV integration within the cell.

Also disclosed are methods for inhibiting HIV integration within a cell, the method comprising the step of contacting the cell an effective amount of a binding agent of phospholipase D (PLD), wherein the binding agent binds to at least one amino acid in a catalytic domain of PLD, thereby inhibiting HIV integration within the cell.

Also disclosed are methods for treating a subject for HIV infection, the method comprising the step of administering to the subject an effective amount of an allosteric binding agent of phospholipase D (PLD), thereby treating the subject for HIV infection.

Also disclosed are methods for inhibiting HIV replication within a cell, the method comprising the step of contacting the cell with an effective amount of an allosteric binding agent of phospholipase D (PLD), thereby inhibiting HIV replication within the cell.

Also disclosed are methods for inhibiting HIV integration within a cell, the method comprising the step of contacting the cell with an effective amount of an allosteric binding agent of phospholipase D (PLD), thereby inhibiting HIV integration within the cell.

Also disclosed are methods for inhibiting HIV replication within a cell, the method comprising the step of contacting the cell with an effective amount of a binding agent of phospholipase D (PLD), wherein the binding agent binds to at least one amino acid residue in a binding domain comprising amino acids 1-505 of PLD1, or the homologous amino acids of PLD2, thereby inhibiting HIV replication within the cell.

Also disclosed are methods for inhibiting HIV integration within a cell, the method comprising the step of contacting the cell with an effective amount of a binding agent of phospholipase D (PLD), wherein the binding agent binds to at least one amino acid residue in a binding domain comprising amino acids 1-505 of PLD1, or the homologous amino acids of PLD2, thereby inhibiting HIV integration within the cell.

Also disclosed are methods for inhibiting HIV replication within a cell, the method comprising the step of contacting the cell with an effective amount of a binding agent of phospholipase D (PLD), wherein the binding agent binds to at least one amino acid residue in a binding domain comprising at least one amino acid of the full-length PLD1, or the homologous amino acids of PLD2, thereby inhibiting HIV replication within the cell.

Also disclosed are methods for inhibiting HIV integration within a cell, the method comprising the step of contacting the cell with an effective amount of a binding agent of phospholipase D (PLD), wherein the binding agent binds to at least one amino acid residue in a binding domain comprising at least one amino acid of the full-length PLD1, or the homologous amino acids of PLD2, thereby inhibiting HIV integration within the cell.

Also disclosed are methods for treating a subject comprising the step of co-administering an effective amount of two or more therapeutic agents to the subject; wherein the subject has been diagnosed with a need for treatment of an HIV infection prior to the administering step; and wherein the combination of two or more therapeutic agents comprises: a) a phospholipase D inhibitor; and b) one or more therapeutic agents selected from: i) an HIV fusion/lysis inhibitor, or a pharmaceutically acceptable prodrug, salt, solvate, or polymorph thereof; ii) an HIV integrase inhibitor, or a pharmaceutically acceptable prodrug, salt, solvate, or polymorph thereof; iii) an HIV non-nucleoside reverse transcriptase inhibitor, or a pharmaceutically acceptable prodrug, salt, solvate, or polymorph thereof; iv) an HIV nucleoside reverse transcriptase inhibitor, or a pharmaceutically acceptable prodrug, salt, solvate, or polymorph thereof; and v) an HIV protease inhibitor, or a pharmaceutically acceptable prodrug, salt, solvate.

Also disclosed are methods for inhibiting HIV replication in at least one cell, comprising the step of contacting the at least one cell with an effective amount of at least one compound having a structure represented by a formula:

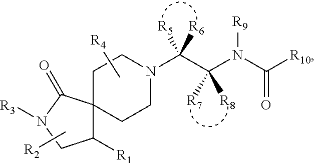

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or ahydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting HIV replication in at least one cell.

Also disclosed are methods for inhibiting HIV replication in at least one cell, comprising the step of contacting the at least one cell with an effective amount of at least one compound having a structure represented by a formula:

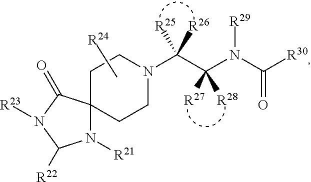

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or ahydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting HIV replication in at least one cell.

Also disclosed are methods for inhibiting HIV replication in at least one cell, comprising the step of contacting the at least one cell with an effective amount of at least one compound having a structure represented by a formula:

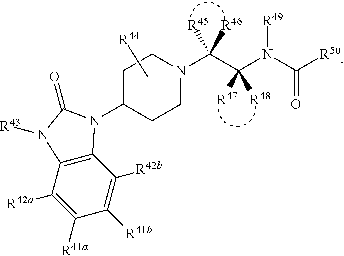

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting HIV replication in at least one cell.

Also disclosed are methods for inhibiting HIV replication in at least one cell, comprising the step of contacting the at least one cell with an effective amount of at least one compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof, thereby inhibiting HIV replication in at least one cell.

Also disclosed are methods for inhibiting HIV replication in at least one cell, comprising the step of contacting the at least one cell with an effective amount of at least one compound having a structure represented by a formula:

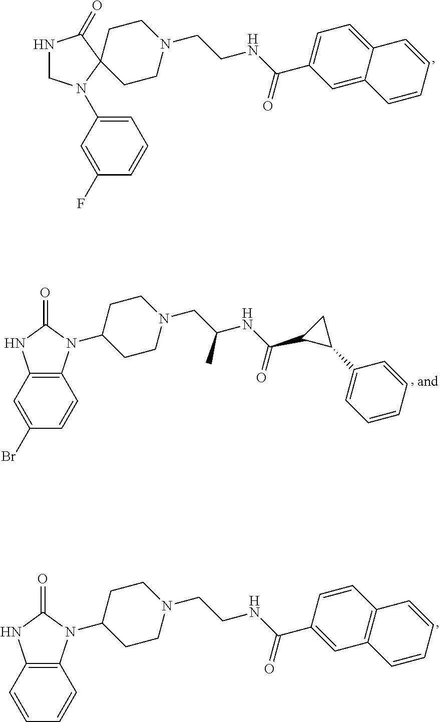

or a pharmaceutically acceptable salt, hydrate, solvate, or polymorph thereof, thereby inhibiting HIV replication in at least one cell.

Also disclosed are methods for decreasing HIV viral load in at least one cell, comprising the step of contacting the at least one cell with an effective amount of at least one compound having a structure represented by a formula:

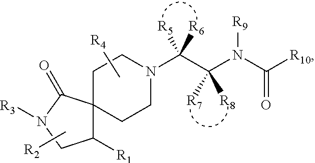

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting HIV replication in at least one cell.

Also disclosed are methods for decreasing HIV viral load in at least one cell, comprising the step of contacting the at least one cell with an effective amount of at least one compound having a structure represented by a formula:

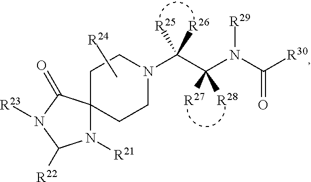

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting HIV replication in at least one cell.

Also disclosed are methods for decreasing HIV viral load in at least one cell, comprising the step of contacting the at least one cell with an effective amount of at least one compound having a structure represented by a formula:

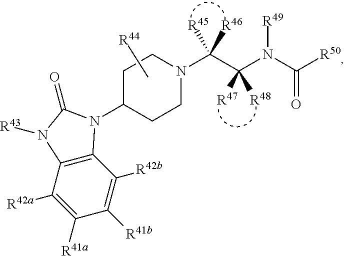

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting HIV replication in at least one cell.

Also disclosed are methods for decreasing HIV viral load in at least one cell, comprising the step of contacting the at least one cell with an effective amount of at least one compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof, thereby inhibiting HIV replication in at least one cell.

Also disclosed are methods for decreasing HIV viral load in at least one cell, comprising the step of contacting the at least one cell with an effective amount of at least one compound having a structure represented by a formula:

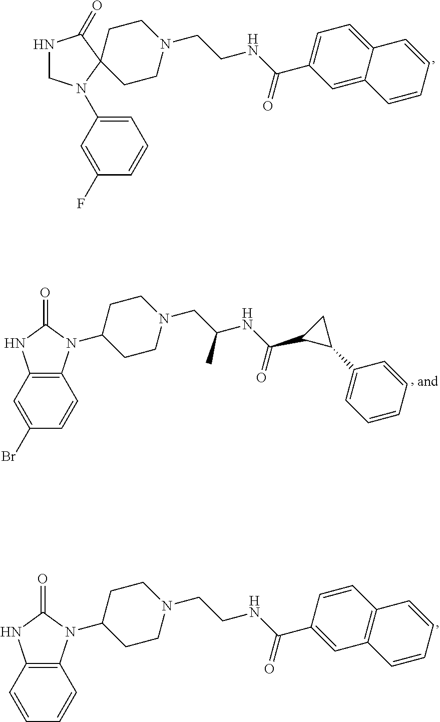

or a pharmaceutically acceptable salt, hydrate, solvate, or polymorph thereof, thereby inhibiting HIV replication in at least one cell.

Also disclosed are methods for decreasing nucleotide pools in at least one cell, comprising the step of contacting the at least one cell with an effective amount of at least one compound having a structure represented by a formula:

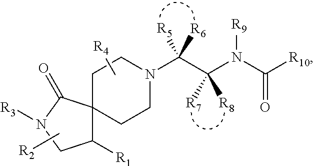

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting HIV replication in at least one cell.

Also disclosed are methods for decreasing nucleotide pools in at least one cell, comprising the step of contacting the at least one cell with an effective amount of at least one compound having a structure represented by a formula:

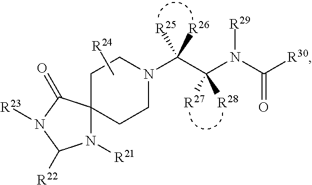

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting HIV replication in at least one cell.

Also disclosed are methods for decreasing nucleotide pools in at least one cell, comprising the step of contacting the at least one cell with an effective amount of at least one compound having a structure represented by a formula:

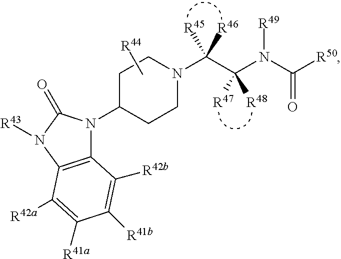

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting HIV replication in at least one cell.

Also disclosed are methods for decreasing nucleotide pools in at least one cell, comprising the step of contacting the at least one cell with an effective amount of at least one compound selected from: a) trans-diethylstilbestrol; b) resveratrol; c) honokiol; d) SCH420789; e) presqualene diphosphate; f) raloxifene; g) 4-hydroxytamoxifen; h) 5-fluoro-2-indoyl des-chlorohalopemide; and i) halopemide, or a pharmaceutically acceptable salt, hydrate, solvate, or polymorph thereof, thereby inhibiting HIV replication in at least one cell.

Also disclosed are methods for decreasing nucleotide pools in at least one cell, comprising the step of contacting the at least one cell with an effective amount of at least one compound having a structure represented by a formula:

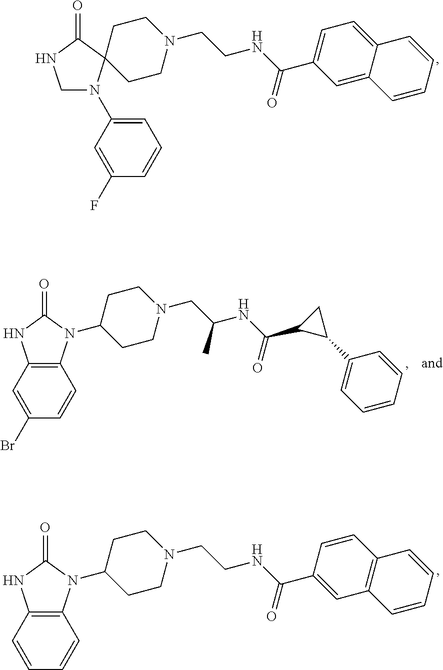

or a pharmaceutically acceptable salt, hydrate, solvate, or polymorph thereof, thereby inhibiting HIV replication in at least one cell.

Also disclosed are pharmaceutical compositions comprising a pharmaceutically acceptable carrier and an effective amount of a disclosed compound, or a pharmaceutically acceptable salt, hydrate, solvate, or polymorph thereof, wherein the pharmaceutical composition is administered for the treatment of an HIV infection.

Also disclosed are kits comprising a phospholipase D inhibitor, or a pharmaceutically acceptable prodrug, salt, solvate, or polymorph thereof, and one or more of: a) at least one agent known to treat an HIV infection; b) at least one agent known to treat an opportunistic infection associated with an HIV infection; c) instructions for treating an HIV infection; d) instructions for treating an opportunistic infection associated with an HIV infection; e) instructions for administering the phospholipase D inhibitor in connection with treating an HIV infection; or f) instructions for administering the phospholipase D inhibitor in connection with reducing the risk of HIV infection.

Also disclosed are methods for manufacturing a medicament comprising combining at least one disclosed compound with a pharmaceutically acceptable carrier or diluent, wherein the medicament is used to treat an HIV infection.

Also disclosed are uses of a disclosed for the treatment of an HIV infection in a mammal.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows representative data pertaining to the effect of representative PLD inhibitors on HIV-1 replication in primary macrophages.

FIG. 2 shows representative data pertaining to the effect of dominant-negative ATG4B on PLD inhibitor-dependent reduction of HIV gag.

FIG. 3 shows representative data pertaining to the effect of SamHD1 on dNTP levels in THP-1 cells.

FIG. 4 shows representative data pertaining to the effect of representative PLD inhibitors on dNTP levels in THP-1 cells.

FIG. 5 shows representative data pertaining to the effect of representative PLD inhibitors on dNTP levels in THP-1 cells.

FIG. 6 shows representative data pertaining to the effect of SamHD1 depletion on dNTP levels in THP-1 cells.

FIG. 7 shows representative data pertaining to the effect of representative PLD inhibitors on HIV-1 infection in THP-1 cells.

FIG. 8 shows representative data pertaining to the effect of representative PLD inhibitors on HIV-1 infection in PMA-stimulated THP-1 cells.

FIG. 9 shows representative data pertaining to the effect of representative PLD inhibitors on HIV-1 infection in PMA-stimulated THP-1 cells.

FIG. 10 shows representative data pertaining to the effect of PLD inhibitors on HIV-2 replication in THP-1 cells.

FIG. 11 shows the role of PLD in HIV-1 replication.

FIG. 12 shows the role of PLD inhibitor treatment on the mTORC1 signaling pathway.

FIG. 13 shows representative data pertaining to PLD-mediated regulation of dNTP levels.

FIG. 14 shows representative data pertaining to the effect of EVJ on HIV-1 replication in activated primary CD4+ T-cells.

FIG. 15 shows that inhibition of the PLD or mTOR pathway reduces HIV-1 infection of PMA-stimulated THP-1 cells.

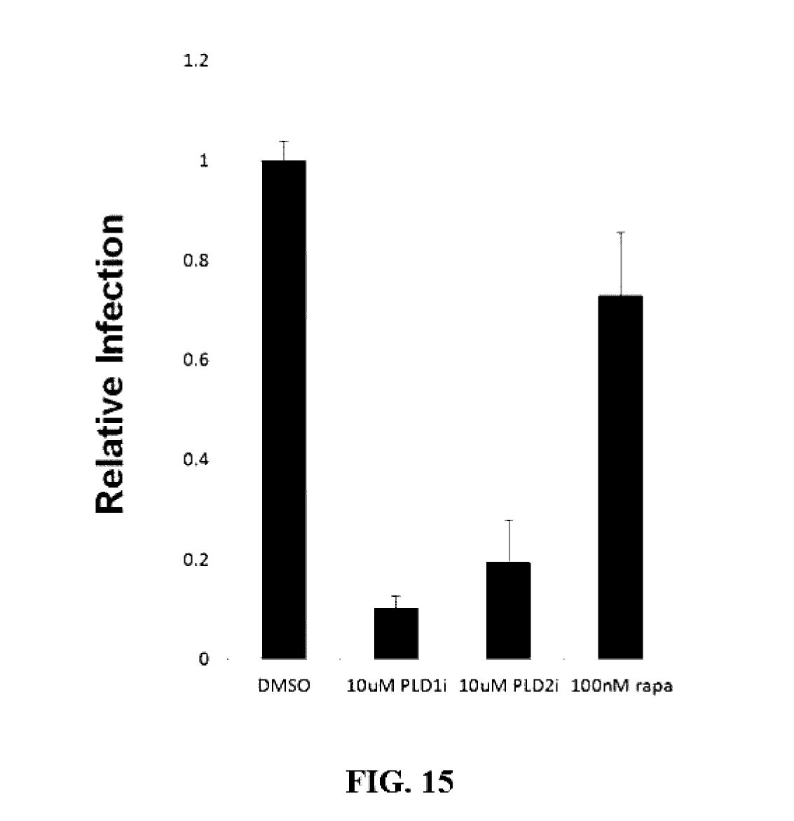

FIG. 16 shows representative data indicating that PLD inhibitors suppress HIV-1 replication in primary macrophages.

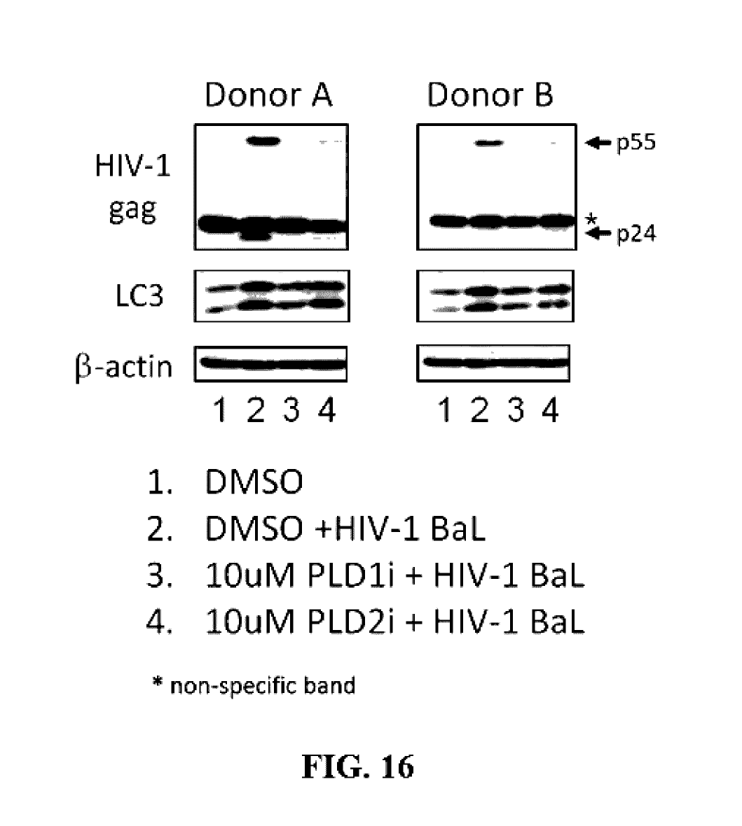

FIG. 17 shows representative data demonstrating that PLD inhibitors synergize with other components of a HIV therapeutic cocktail.

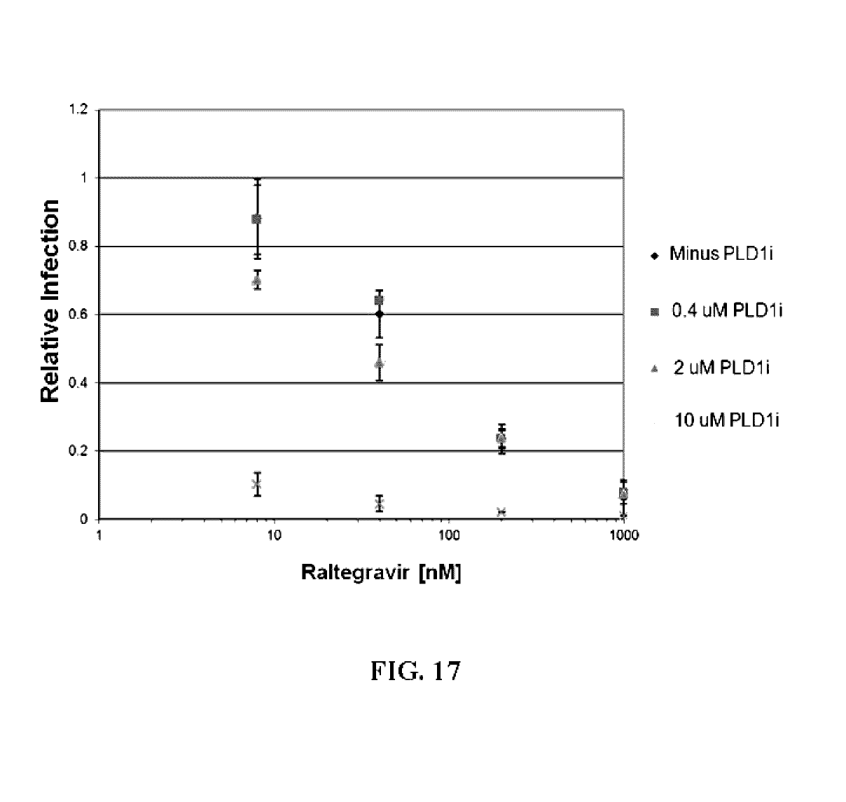

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about,” “approximate,” and “at or about” mean that the amount or value in question can be the exact value designated or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

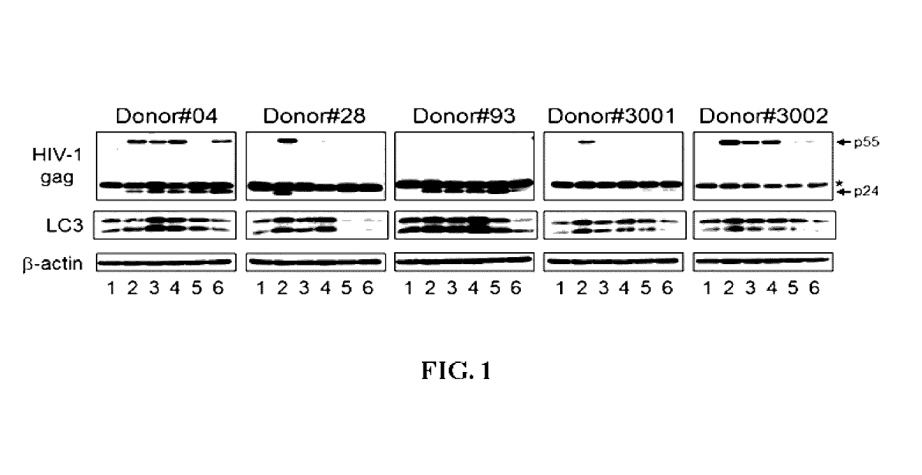

As used herein, the terms “phospholipase D” and “PLD” can be used interchangeably, and refer to a protein family comprising at least the following members: PLD1 and PLD2. Activation of PLDs occurs as a consequence of agonist stimulation of both tyrosine kinase and G protein-coupled receptors. PC-specific PLDs have been proposed to function in regulated secretion, cytoskeletal reorganization, transcriptional regulation, and cell cycle control. PLDs may also be involved in the regulation of perinuclear intravesicular membrane traffic. Several domains are described for the protein, with the overall primary domain structure for PLD1 as given in FIG. 1. PLD2 lacks the “loop” domain, but otherwise has the same domains located at about the same relative positions in the protein.

The PLD protein family catalyzes a variety of reaction. The most well-characterized reaction is the hydrolysis of phosphatidylcholine to produce phosphatidic acid and choline, as follows:

a phosphatidylcholine+H₂O→choline+a phosphatidate

Although the foregoing is the most well-characterized reaction catalyzed by PLD, there are additional reactions which are also catalyzed by PLD. These reactions include:

a lysophosphatidylcholine+H₂O→choline+a lysophosphatidate

a lysophosphotidylcholine→choline+a cyclic lysophosphotidate

a phosphotidylcholine+ROH→choline+a phosphotidylalcohol

The reactions catalyzed by PLD can involve headgroups other than choline. For example, hydrolysis of the headgroup can be generalized as follows:

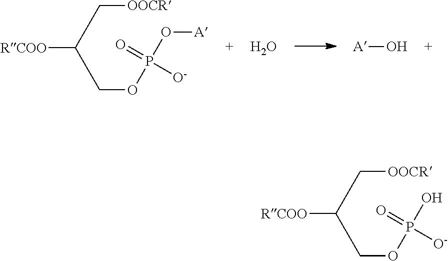

In the foregoing reaction scheme, the R′COO and R″COO moieties derive from fatty acids, e.g. C₁₆-C₂₂ saturated and unsaturated fatty acids (including polyenoic acids). It should be understood that A′ represents an amine containing moiety, e.g. choline.

Alternatively, PLD can also catalyze a transphosphatidylation reaction as follows:

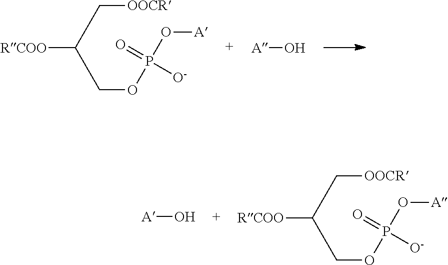

In the foregoing reaction scheme, the R′COO, R″COO, and A′ moieties have the same meaning as in the previous reaction. In addition, the A″-OH moiety represents is a primary alcohol.

As used herein, the terms “phospholipase D1” and “PLD1” refer to the phospholipase D1 protein encoded by a gene designated in human as the PLD1 gene, which has a human gene map locus described by Entrez Gene cytogenetic band: 3q26; Ensembl cytogenetic band: 3q26.31; and, HGNC cytogenetic band: 3q26. The term PLD1 refers to a human protein that has about 1074 amino acids and has a molecular weight of about 124,184 Da. The term is inclusive of splice isoforms or mRNA transcript variants, e.g. the alternative mRNA splicing products that code for the isoforms designated as PLD1A, PLD1B, PLD1C, and PLD1D. The term is also inclusive of that protein referred to by such alternative designations as: “PLD1”, “phospholipase D1, phosphatidylcholine-specific”, “choline phosphatase 1”, “phosphatidylcholine-hydrolyzing phospholipase D1”, PLD1″, “PLD 1”, “EC 3.1.4.4”, “phospholipase D1”, and “phospholipase D1, phosphatidylcholine-specific”, as used by those skilled in the art to refer to that protein encoded by human gene PLD1 or to the gene itself. The term is also inclusive of the non-human orthologs or homologs thereof, as well as splice variants and alternative transcripts of the PLD1 gene.

As used herein, the terms “phospholipase D2” and “PLD2” refer to the phospholipase D2 protein encoded by a gene designated in human as the PLD2 gene, which has a human gene map locus described by Entrez Gene cytogenetic band: 17p13.1; Ensembl cytogenetic band: 17p13.2; and, HGNC cytogenetic band: 17p13.3. The term PLD2 refers to a human protein that has about 933 amino acids and has a molecular weight of about 105,987 Da. The term is inclusive of splice isoforms or mRNA transcript variants, e.g. the alternative mRNA splicing products that code for the isoforms designated as PLD2A, PLD2B, and PLD2C. The term is also inclusive of that protein referred to by such alternative designations as: “PLD2”, “phospholipase D2”, “Choline phosphatase 2”, “Phosphatidylcholine-hydrolyzing phospholipase D2”, “PLD1C”, “hPLD2”, “PLD 2”, and “EC 3.1.4.4”, as used by those skilled in the art to refer to that protein encoded by human gene PLD2 or to the gene itself. The term is also inclusive of the non-human orthologs or homologs thereof, as well as splice variants and alternative transcripts of the PLD2 gene.

As used herein, the term “PLD inhibitor” refers to any exogenously administered compound or agent that directly inhibits the activity of a PLD gene product. In this context, an inhibitor is understood to directly decrease the activity of the target PLD gene product compared to the activity of the gene product in the absence of the exogenously administered compound or agent. Examples of directly acting compounds or agents are allosteric inhibitors, competitive inhibitors, noncompetitive inhibitors, irreversible inhibitors, and uncompetitive inhibitors. In addition, PLD inhibitor is understood to include agents or compounds that decrease the activity of the target PLD gene product compared to the activity of the gene product in the absence of the exogenously administered compound or agent via a indirect mechanisms, e.g. without binding directly to the target PLD gene product. For example, the compound honokiol has the effect of inhibiting the activity of PLD without directly binding to PLD. It is known to one skilled in the art that honokiol acts on the ras-Rhoa complex to inhibit expression of PLD, and thus decrease the level of PLD activity in cells. Resveratrol is another example of a compound that has the effect of inhibiting PLD in cells without directly binding to the target PLD gene product.

As used herein, the term “PLD1 inhibitor” refers to any exogenously administered compound or agent that directly inhibits the activity of a PLD1 gene product. In this context, an inhibitor is understood to directly decrease the activity of the target PLD1 gene product compared to the activity of the gene product in the absence of the exogenously administered compound or agent. Examples of directly acting compounds or agents are allosteric inhibitors, competitive inhibitors, noncompetitive inhibitors, irreversible inhibitors, and uncompetitive inhibitors.

As used herein, the term “PLD2 inhibitor” refers to any exogenously administered compound or agent that directly inhibits the activity of a PLD2 gene product. In this context, an inhibitor is understood to directly decrease the activity of the target PLD2 gene product compared to the activity of the gene product in the absence of the exogenously administered compound or agent. Examples of directly acting compounds or agents are allosteric inhibitors, competitive inhibitors, noncompetitive inhibitors, irreversible inhibitors, and uncompetitive inhibitors.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein.

As used herein, “gene product” refers to transcription or translation products that are derived from a specific gene locus or gene. The “gene locus” or “gene” includes coding sequences as well as regulatory, flanking and intron sequences.

The term “HIV infection” refers to the introduction of HIV into cells or tissues. In general, the introduction of HIV is also associated with replication. HIV infection may be determined by measuring HIV antibody titer in samples of a biological fluid, such as blood, using, e.g., enzyme immunoassay. Other suitable diagnostic methods include molecular based techniques, such as RT-PCR, direct hybrid capture assay, nucleic acid sequence based amplification, and the like. A virus may infect an particular organ, e.g., lung, and cause disease, e.g., localized effects such as respiratory impairment and edema, and systemic effects.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder, e.g. an infection with HIV. The term “patient” includes human and veterinary subjects. In some aspects of the disclosed methods, the subject has been diagnosed with a need for treatment of one or more HIV infections prior to the administering step. In some aspects of the disclosed method, the subject has been diagnosed with a need for inhibition of PLD1, PLD2, or both PLD1 and PLD2 activity prior to the administering step. In some aspects of the disclosed method, the subject has been diagnosed with an HIV infection. In some aspects of the disclosed method, the subject has been identified with a disorder treatable by inhibition of PLD1, PLD2, or both PLD1 and PLD2 activity prior to the administering step. In one aspect, a subject can be treated prophylactically with a compound or composition disclosed herein, as discussed herein elsewhere. It is understood that a subject can be a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; supportive treatment, that is, treatment employed to supplement another specific therapy directed toward the improvement of the associated disease, pathological condition, or disorder; and prophylactic treatment, that is, treatment directed to preventing a disease or disorder in a subject, preventing the occurrence of symptoms in a subject with a disease or disorder, preventing the recurrence of symptoms in a subject with a disease or disorder, and/or decreasing the severity of frequency of outward symptoms of disease or disorder in a subject. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

As used herein, the term “prophylaxis” refers to the complete prevention of infection, the prevention of occurrence of symptoms in an infected subject, the prevention of recurrence of symptoms in an infected subject, or a decrease in severity or frequency of outward symptoms of HIV infection or disease in the subject.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a disorder treatable by selective inhibition of Phospholipase D1” means having been subjected to a physical examination by a person of skill, for example, a physician, and found to have a condition that can be diagnosed or treated by a compound or composition that can inhibit PLD1. As a further example, “diagnosed with a need for selective inhibition of Phospholipase D2” refers to having been subjected to a physical examination by a person of skill, for example, a physician, and found to have a condition characterized by PLD2 activity. Such a diagnosis can be in reference to a disorder, such as a disease of uncontrolled cellular proliferation, and the like, as discussed herein.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to PLD2 activity) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The terms “co-administer(s)”, “co-administering”, and “co-administration” all refer to with respect to compounds or compositions, is meant either simultaneous administration or any manner of separate sequential administration of one or more PLD inhibitor compounds, e.g. a PLD1 selective inhibitor, a PLD2 selective inhibitor, or a non-selective inhibitor of PLD1 and PLD2, with one or more pharmaceutically active agents, such as, but not limited to, those agents included in antiviral therapy. Preferably, if the administration is not simultaneous, the compounds are administered in a close time proximity to each other. Furthermore, it does not matter if the compounds are administered in the same dosage form, e.g. one compound may be administered topically and another compound may be administered orally. “Substantially simultaneously” means that the compound, i.e. a PLD inhibitor compound, is typically administered during or within a reasonably short time either before or after the administration of other compounds, such as a pharmaceutically active agent that treats the disease in question. Additionally, “co-administration”, “co-administer(s)”, and “co-administering” include administering more than one dose of the pharmaceutically active agent within 24 hours after a dose of a PLD inhibitor compound. In other words, PLD inhibitors need not be administered again before or with every administration of a pharmaceutically active agent, but may be administered intermittently during the course of treatment. “Co-administration”, “co-administer(s)”, and “co-administering” also includes administering a pharmaceutically active agent and a PLD inhibitor compound as a part of one or more pharmaceutical compositions, and such one or more pharmaceutical compositions may contain a co-formulation of a PLD inhibitor compound and a pharmaceutically active agent or individual formulations of a pharmaceutically active agent and a PLD inhibitor compound.

It is understood that co-administration a PLD inhibitor compound and an anti-HIV agent or other therapeutic agent can be independently co-administered by any appropriate route of administration. The active agents, i.e. a PLD inhibitor compound and an anti-HIV agent or other therapeutic agent, can be administered by the same or different routes of administration, as appropriate. For example, one of the active ingredients can be administered orally and the other administered orally or by some other appropriate route of administration. Alternatively, the combination of active ingredients can be concurrently orally administered. In a further example, consistent with this understanding, one of the active ingredients can be administered parenterally, for example, intravenously, intramuscularly, subcutaneously, topically, intravaginally, rectally, intranasally, inhalationally, intrathecally, intraocularly, and one or more of the other active ingredients administrated by a similar or distinct route of administration. Moreover, it is understood, that a PLD inhibitor compound and an anti-HIV agent or other therapeutic agent can be co-administered or independently administered by distinct routes of administration such as parenterally, orally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, or intrathecally.

As used herein, “combination therapy” (or “co-therapy”) refers to the administration of a PLD inhibitor compound and an anti-HIV agent or other therapeutic agent during the course of therapy or treatment for an HIV infection. Such combination therapy may involve the administration of the PLD inhibitor compound before, during, and/or after the administration of the anti-HIV agent or other therapeutic agent administered to ameliorate, treat, reverse, or cure the HIV infection or symptoms associated with the HIV infection. The administration of the PLD inhibitor compound may be separated in time from the administration of anti-HIV agent or other therapeutic agent by up to several weeks, and may precede it or follow it, but more commonly the administration of the PLD inhibitor compound will accompany at least one aspect of the administration of the anti-HIV agent or other therapeutic agent.

As used herein, “concurrently” means (1) simultaneously in time, or (2) at different times during the course of a common treatment schedule.

The term “contacting” as used herein refers to bringing a disclosed compound and a cell, target histamine receptor, or other biological entity together in such a manner that the compound can affect the activity of the target (e.g., spliceosome, cell, etc.), either directly; i.e., by interacting with the target itself, or indirectly; i.e., by interacting with another molecule, co-factor, factor, or protein on which the activity of the target is dependent.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount or dosage that can effectively prevent a disease or disorder in a subject, prevent the occurrence of symptoms in a subject with a disease or disorder, prevent the recurrence of symptoms in a subject with a disease or disorder, and/or decrease the severity of frequency of outward symptoms of a disease or disorder in a subject.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug,” as used herein means a compound that is metabolized, for example hydrolyzed or oxidized, in the host to form the compound of the present invention without forming fragments with toxicological liabilities. Typical examples of prodrugs include compounds that have biologically labile protecting groups linked to a functional moiety of the active compound. For example, a prodrug can comprise alkylation, acylation or other lipophilic modification of one or more hydroxy group(s) present in a compound of the invention, e.g. a PLD inhibitor compound. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

The term “excipient” as used herein refers to a compound that is used to prepare a pharmaceutical composition, and is generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

The term “immune modulator” refers to any substance meant to alter the working of the humoral or cellular immune system of a subject. Such immune modulators include inhibitors of mast cell-mediated inflammation, interferons, interleukins, prostaglandins, steroids, corticosteroids, colony-stimulating factors, chemotactic factors, etc.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the terms “optional” or “optionally” means that the subsequently described event or circumstance may or may not occur, and that the description includes instances where said event or circumstance occurs and instances where it does not.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “aliphatic” refers to a non-aromatic carbon-based moiety. Aliphatic can include both acyclic or cyclic moieties (e.g., alkyl and cycloalkyl) and can include both saturated and unsaturated moieties (e.g., alkyl, alkenyl, and alkynyl).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of from 1 to 24 carbon atoms, for example from 1 to 12 carbons, from 1 to 8 carbons, from 1 to 6 carbons, or from 1 to 4 carbons, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The cycloalkyl group can be substituted or unsubstituted. The cycloalkyl group can be substituted with one or more groups including, but not limited to, alkyl, cycloalkyl, alkoxy, amino, ether, halide, hydroxy, nitro, silyl, sulfo-oxo, or thiol as described herein.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein. A specific example of amino is —NH₂.

The term “carboxylic acid” as used herein is represented by the formula —C(O)OH.

The term “ester” as used herein is represented by the formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, pyrazolopyrimidinyl, 1,2-oxazol-4-yl, 1,2-oxazol-5-yl, 1,3-oxazolyl, 1,2,4-oxadiazol-5-yl, 1,2,3-triazolyl, 1,3-thiazol-4-yl, pyridinyl, and pyrimidin-5-yl.

The term “heterocycle” as used herein refers to single and multi-cyclic aromatic or non-aromatic ring systems in which at least one of the ring members is other than carbon. Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “heterocycloalkyl” as used herein is a non-aromatic carbon-based ring composed of at least two carbon atoms and at least one non-carbon heteroatom. For example, the non-carbon heteroatom can include, but is not limited to, oxygen, nitrogen, sulphur, phosphorus and the like. Examples of heterocycloalkyl groups include, aziridine, oxirane, thiirane, azetidine, oxetane, thietane, pyrrolidine, tetrahydrofuran, tetrahydrothiophene, piperidine, tetrahydro-2H-pyran, tetrahydro-2H-thipyran, azepane, oxepane, thiepane, azocane, oxocane, thiocane, pyrazolidine, imidazolidine, diazetidine, hexahydropyridazine, piperazine, diazepane, oxazinane, oxazepane, oxazolidine, oxazetine, and the like. The heterocycloalkyl group can be substituted or unsubstituted. The heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “hydroxyl” as used herein is represented by the formula —OH.

The term “ketone” as used herein is represented by the formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification, “S(O)” is a short hand notation for S═O. The term “sulfonyl” is used herein to refer to the sulfo-oxo group represented by the formula —S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by the formula —SH.

The term “organic residue” defines a carbon containing residue, i.e., a residue comprising at least one carbon atom, and includes but is not limited to the carbon-containing groups, residues, or radicals defined herein above. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure

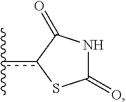

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

In some aspects, a structure of a compound can be represented by a formula:

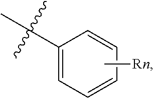

which is understood to be equivalent to a formula:

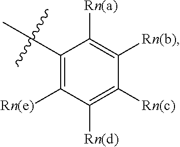

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain instances of the above defined terms may occur more than once in the structural formulae, and upon such occurrence each term shall be defined independently of the other.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “hydrolysable residue” is meant to refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (ee). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### B. PHOSPHOLIPASE D INHIBITORS

In one aspect, the invention relates to compounds, or pharmaceutically acceptable derivatives thereof, useful as isoform selective phospholipase D inhibitors. In general, it is contemplated that each disclosed compound or derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

In one aspect, the compounds of the invention are useful in the treatment of HIV infection. In a further aspect, the compounds are useful in the treatment of disease associated with an HIV infection.

1. Compounds Useful in the Disclosed Methods, Uses, and Pharmaceutical Compositions

In one aspect, the invention relates to phospholipase D inhibitors comprising a compound with a structure represented by a formula:

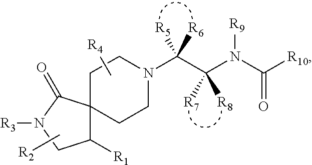

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, trifluoromethyl, carboxamido, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof.

In a further aspect, the compound has a structure represented by a formula:

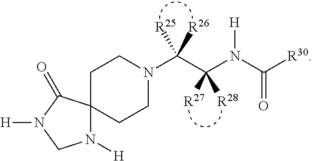

In one aspect, the invention relates to phospholipase D inhibitors comprising a compound with a structure represented by a formula:

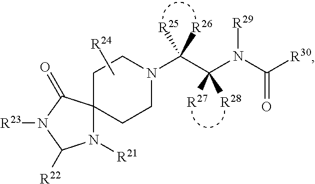

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, trifluoromethyl, carboxamido, alkylsulfonyl, an optionally substituted C1 to C6 alkyl, or an optionally substituted C3 to C6 cycloalkyl or R²⁵ and R²⁶, together with the intermediate carbon, comprise an optionally substituted C3 to C6 cycloalkyl; wherein each of R²⁷ and R²⁸ independently comprises hydrogen, trifluoromethyl, carboxamido, alkylsulfonyl, an optionally substituted C1 to C6 alkyl, or an optionally substituted C3 to C6 cycloalkyl or R²⁷ and R²⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof.

In a further aspect, the compound has a structure represented by a formula:

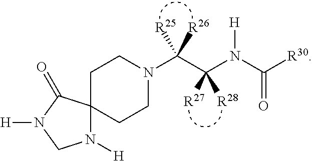

In one aspect, the invention relates to phospholipase D inhibitors comprising a compound with a structure represented by a formula:

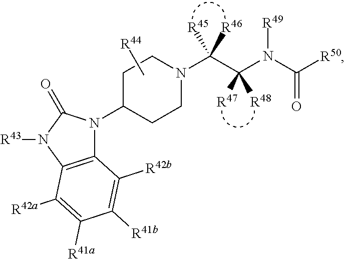

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, trifluoromethyl, carboxamido, alkylsulfonyl, an optionally substituted C1 to C6 alkyl, or an optionally substituted C3 to C6 cycloalkyl or R⁴⁵ and R⁴⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁴⁷ and R⁴⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the compound has a structure represented by a formula:

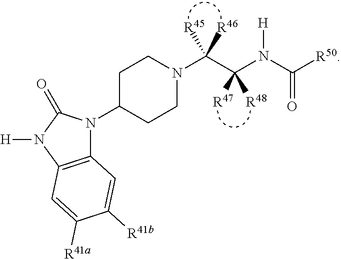

It is understood that the disclosed compounds can be used in connection with the disclosed methods, compositions, kits, and uses.

2. R¹ Groups

In one aspect, R¹ is an optionally substituted C3 to C9 organic residue selected from aryl, heteroaryl, cycloalkyl, heterocycloalkyl, cycloalkenyl, and heterocycloalkenyl.

In a further aspect, R¹ is optionally substituted aryl selected from phenyl and naphthyl.

In a further aspect, R¹ is optionally substituted heteroaryl selected from furanyl, pyranyl, imidazolyl, thiophenyl, pyridinyl, pyridazinyl, pyrimidinyl, pyrazinyl, triazinyl, tetrazinyl, benzofuranyl, benzothiophenyl, indolyl, indazolyl, quinolinyl, naphthyridinyl, benzothiazolyl, benzooxazolyl, benzoimidazolyl, and benzotriazolyl.

In a further aspect, R¹ is optionally substituted cycloalkyl selected from cyclopropyl, cyclobutyl, cyclopentyl, cyclohexyl, cycloheptyl, cyclooctyl, cyclononyl, bicyclo[3.1.0]hexyl, bicyclo[4.1.0]heptyl, bicyclo[5.1.0]octyl, bicyclo[6.1.0]nonyl, bicyclo[3.2.0]heptyl, bicyclo[4.2.0]octyl, bicyclo[5.2.0]nonyl, bicyclo[3.3.0]octyl, bicyclo[4.3.0]nonyl, bicyclo[2.2.1]heptyl, bicyclo[3.2.1]octyl, bicyclo[4.2.1]nonyl, bicyclo[2.2.2]octyl, bicyclo[3.2.2]nonyl, and bicyclo[3.3.1]nonyl.

In a further aspect, R¹ is optionally substituted heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R¹ is optionally substituted cycloalkenyl selected from cyclobutenyl, cyclopentenyl, cyclopentadienyl, cyclohexenyl, cyclohexadienyl, cycloheptenyl, cycloheptadienyl, cyclooctenyl, cyclooctadienyl, cyclononenyl, and cyclononadienyl.

In a further aspect, R¹ is optionally substituted heterocycloalkenyl comprising a mono-, di- or tri-unsaturated analog of a heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R¹ is halophenyl, for example 4-fluorophenyl.

3. R² Groups

In one aspect, R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each R² is hydrogen. In a further aspect, each R² is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each R² is independently selected from halide, hydroxyl, trifluoromethyl, amino, cyano, nitro, azide, carboxamido, alkoxy, thiol, and alkylsulfonyl. In a further aspect, at least one R² is methyl, ethyl, n-propyl, i-propyl, cyclopropyl, n-butyl, butyl, s-butyl, cyclobutyl, n-pentyl, i-pentyl, s-pentyl, neopentyl, cyclopentyl, n-hexyl, hexyl, s-hexyl, dimethylbutyl, or cyclohexyl.

4. R³ Groups

In one aspect, R³ comprises hydrogen, an optionally substituted C1 to C6 alkyl, an optionally substituted C3 to C6 cycloalkyl, or a hydrolysable residue.

In a further aspect, R³ is hydrogen. In a further aspect, R³ is an optionally substituted C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl. In a further aspect, R³ is an optionally substituted C3 to C6 cycloalkyl selected from cyclopropyl, cyclobutyl, cyclopentyl, cyclohexyl, and bicyclo[3.1.0]hexyl. In a further aspect, R³ is a hydrolysable residue.

5. R⁴ Groups

In one aspect, R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each R⁴ is hydrogen. In a further aspect, each R⁴ is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each R⁴ is independently selected from halide, hydroxyl, trifluoromethyl, amino, cyano, nitro, azide, carboxamido, alkoxy, thiol, and alkylsulfonyl. In a further aspect, at least one R⁴ is methyl, ethyl, n-propyl, i-propyl, cyclopropyl, n-butyl, butyl, s-butyl, cyclobutyl, n-pentyl, i-pentyl, s-pentyl, neopentyl, cyclopentyl, n-hexyl, hexyl, s-hexyl, dimethylbutyl, or cyclohexyl.

6. R⁵ and R⁶ Groups

In one aspect, each of R⁵ and R⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl.

In a further aspect, R⁵ is hydrogen. In a further aspect, R⁵ is selected from trifluoromethyl, carboxamido, alkylsulfonyl, and an optionally substituted C1 to C6 organic residue. In a further aspect, R⁵ is selected from trifluoromethyl, carboxamido, and alkylsulfonyl. In a further aspect, R⁵ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁶ is hydrogen. In a further aspect, R⁶ is selected from trifluoromethyl, carboxamido, alkylsulfonyl, and an optionally substituted C1 to C6 organic residue. In a further aspect, R⁶ is selected from trifluoromethyl, carboxamido, and alkylsulfonyl. In a further aspect, R⁶ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁶ is hydrogen and wherein R⁵ is selected from trifluoromethyl, carboxamido, alkylsulfonyl, and an optionally substituted C1 to C6 organic residue. In a further aspect, R⁶ is hydrogen and wherein R⁵ is selected from trifluoromethyl, carboxamido, and alkylsulfonyl. In a further aspect, R⁶ is hydrogen and wherein R⁵ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁵ is hydrogen and wherein R⁶ is selected from trifluoromethyl, carboxamido, alkylsulfonyl, and an optionally substituted C1 to C6 organic residue. In a further aspect, R⁵ is hydrogen and wherein R⁶ is selected from trifluoromethyl, carboxamido, and alkylsulfonyl. In a further aspect, R⁵ is hydrogen and wherein R⁶ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁵ and R⁶, together with the intermediate carbon, comprise an optionally substituted C3 to C6 cycloalkyl. In a further aspect, wherein R⁵ and R⁶, together with the intermediate carbon, comprise cyclopropyl, cyclobutyl, cyclopentyl, or cyclohexyl.

7. R⁷ and R⁸ Groups

In one aspect, each of R⁷ and R⁸ independently comprises hydrogen, trifluoromethyl, carboxamido, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl.

In a further aspect, R⁷ is hydrogen. In a further aspect, R⁷ is selected from trifluoromethyl, carboxamido, alkylsulfonyl, and an optionally substituted C1 to C6 organic residue. In a further aspect, R⁷ is selected from trifluoromethyl, carboxamido, and alkylsulfonyl. In a further aspect, R⁷ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl. In a further aspect, R⁷ is methyl.

In a further aspect, R⁸ is hydrogen. In a further aspect, R⁸ is selected from trifluoromethyl, carboxamido, alkylsulfonyl, and an optionally substituted C1 to C6 organic residue. In a further aspect, R⁸ is selected from trifluoromethyl, carboxamido, and alkylsulfonyl. In a further aspect, R⁸ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl. In a further aspect, R⁸ is methyl.

In a further aspect, R⁸ is hydrogen and wherein R⁷ is selected from trifluoromethyl, carboxamido, alkylsulfonyl, and an optionally substituted C1 to C6 organic residue. In a further aspect, R⁸ is hydrogen and wherein R⁷ is selected from trifluoromethyl, carboxamido, and alkylsulfonyl. In a further aspect, R⁸ is hydrogen and wherein R⁷ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁷ is hydrogen and wherein R⁸ is selected from trifluoromethyl, carboxamido, alkylsulfonyl, and an optionally substituted C1 to C6 organic residue. In a further aspect, R⁷ is hydrogen and wherein R⁸ is selected from trifluoromethyl, carboxamido, and alkylsulfonyl. In a further aspect, R⁷ is hydrogen and wherein R⁸ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁷ and R⁸, together with the intermediate carbon, comprise an optionally substituted C3 to C6 cycloalkyl. In a further aspect, R⁷ and R⁸, together with the intermediate carbon, comprise cyclopropyl, cyclobutyl, cyclopentyl, or cyclohexyl.

8. R⁹ Groups

In one aspect, R⁹ comprises hydrogen, an optionally substituted C1 to C6 alkyl, an optionally substituted C3 to C6 cycloalkyl, or a hydrolysable residue.

In a further aspect, R⁹ is hydrogen. In a further aspect, R⁹ is an optionally substituted C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl. In a further aspect, R⁹ is an optionally substituted C3 to C6 cycloalkyl selected from cyclopropyl, cyclobutyl, cyclopentyl, and cyclohexyl. In a further aspect, R⁹ is a hydrolysable residue.

9. R¹⁰ Groups

In one aspect, R¹⁰ comprises an optionally substituted C1 to C12 organic residue selected from alkyl, aryl, heteroaryl, cycloalkyl, heterocycloalkyl, cycloalkenyl, and heterocycloalkenyl.

In a further aspect, R¹⁰ is an optionally substituted alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, cyclohexyl, heptyl, cycloheptyl, octyl, cyclooctyl, nonyl, cyclononyl, decyl, cyclodecyl, undecyl, cycloundecyl, dodecyl, or cyclododecyl.

In a further aspect, R¹⁰ is an optionally substituted aryl selected from phenyl and naphthyl.

In a further aspect, R¹⁰ is an optionally substituted heteroaryl selected from furanyl, pyranyl, imidazolyl, thiophenyl, pyridinyl, pyridazinyl, pyrimidinyl, pyrazinyl, triazinyl, tetrazinyl, benzofuranyl, benzothiophene, indolyl, indazolyl, quinolinyl, naphthyridinyl, benzothiazolyl, benzooxazolyl, benzoimidazolyl, and benzotriazolyl.

In a further aspect, R¹⁰ is an optionally substituted cycloalkyl selected from cyclopropyl, cyclobutyl, cyclopentyl, cyclohexyl, cycloheptyl, cyclooctyl, cyclononyl, bicyclo[3.1.0]hexyl, bicyclo[4.1.0]heptyl, bicyclo[5.1.0]octyl, bicyclo[6.1.0]nonyl, bicyclo[3.2.0]heptyl, bicyclo[4.2.0]octyl, bicyclo[5.2.0]nonyl, bicyclo[3.3.0]octyl, bicyclo[4.3.0]nonyl, bicyclo[2.2.1]heptyl, bicyclo[3.2.1]octyl, bicyclo[4.2.1]nonyl, bicyclo[2.2.2]octyl, bicyclo[3.2.2]nonyl, and bicyclo[3.3.1]nonyl.

In a further aspect, R¹⁰ is an optionally substituted heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R¹⁰ is optionally substituted cycloalkenyl selected from cyclobutenyl, cyclopentenyl, cyclopentadienyl, cyclohexenyl, cyclohexadienyl, cycloheptenyl, cycloheptadienyl, cyclooctenyl, cyclooctadienyl, cyclononenyl, and cyclononadienyl.

In a further aspect, R¹⁰ is optionally substituted heterocycloalkenyl comprising a mono-, di- or tri-unsaturated analog of a heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R¹⁰ is phenylethynyl, indolyl, quinolinyl, naphthyl, phenylcyclopropyl, or fluorophenyl.

10. R²¹ Groups

In one aspect, R²¹ is an optionally substituted C3 to C9 organic residue selected from aryl, heteroaryl, cycloalkyl, heterocycloalkyl, cycloalkenyl, and heterocycloalkenyl.

In a further aspect, R²¹ is optionally substituted aryl selected from phenyl and naphthyl.

In a further aspect, R²¹ is optionally substituted heteroaryl selected from furanyl, pyranyl, imidazolyl, thiophenyl, pyridinyl, pyridazinyl, pyrimidinyl, pyrazinyl, triazinyl, tetrazinyl, benzofuranyl, benzothiophene, indolyl, indazolyl, quinolinyl, naphthyridinyl, benzothiazolyl, benzooxazolyl, benzoimidazolyl, and benzotriazolyl.

In a further aspect, R²¹ is optionally substituted cycloalkyl selected from cyclopropyl, cyclobutyl, cyclopentyl, cyclohexyl, cycloheptyl, cyclooctyl, cyclononyl, bicyclo[3.1.0]hexyl, bicyclo[4.1.0]heptyl, bicyclo[5.1.0]octyl, bicyclo[6.1.0]nonyl, bicyclo[3.2.0]heptyl, bicyclo[4.2.0]octyl, bicyclo[5.2.0]nonyl, bicyclo[3.3.0]octyl, bicyclo[4.3.0]nonyl, bicyclo[2.2.1]heptyl, bicyclo[3.2.1]octyl, bicyclo[4.2.1]nonyl, bicyclo[2.2.2]octyl, bicyclo[3.2.2]nonyl, and bicyclo[3.3.1]nonyl.

In a further aspect, R²¹ is optionally substituted heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R²¹ is optionally substituted cycloalkenyl selected from cyclobutenyl, cyclopentenyl, cyclopentadienyl, cyclohexenyl, cyclohexadienyl, cycloheptenyl, cycloheptadienyl, cyclooctenyl, cyclooctadienyl, cyclononenyl, and cyclononadienyl.

In a further aspect, R²¹ is optionally substituted heterocycloalkenyl comprising a mono-, di- or tri-unsaturated analog of a heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R²¹ is halophenyl, for example 4-fluorophenyl.

11. R²² Groups

In one aspect, R²² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each R²² is hydrogen. In a further aspect, each R²² is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each R²² is independently selected from halide, hydroxyl, trifluoromethyl, amino, cyano, nitro, azide, carboxamido, alkoxy, thiol, and alkylsulfonyl. In a further aspect, at least one R²² is methyl, ethyl, n-propyl, i-propyl, cyclopropyl, n-butyl, butyl, s-butyl, cyclobutyl, n-pentyl, i-pentyl, s-pentyl, neopentyl, cyclopentyl, n-hexyl, hexyl, s-hexyl, dimethylbutyl, or cyclohexyl.

12. R²³ Groups

In one aspect, R²³ comprises hydrogen, an optionally substituted C1 to C6 alkyl, an optionally substituted C3 to C6 cycloalkyl, or a hydrolysable residue.

In a further aspect, R²³ is hydrogen. In a further aspect, R²³ is an optionally substituted C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl. In a further aspect, R²³ is an optionally substituted C3 to C6 cycloalkyl selected from cyclopropyl, cyclobutyl, cyclopentyl, cyclohexyl, and bicyclo[3.1.0]hexyl. In a further aspect, R²³ is a hydrolysable residue.

13. R²⁴ Groups

In one aspect, R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each R²⁴ is hydrogen. In a further aspect, each R²⁴ is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each R²⁴ is independently selected from halide, hydroxyl, trifluoromethyl, amino, cyano, nitro, azide, carboxamido, alkoxy, thiol, and alkylsulfonyl. In a further aspect, at least one R²⁴ is methyl, ethyl, n-propyl, i-propyl, cyclopropyl, n-butyl, butyl, s-butyl, cyclobutyl, n-pentyl, i-pentyl, s-pentyl, neopentyl, cyclopentyl, n-hexyl, hexyl, s-hexyl, dimethylbutyl, or cyclohexyl.

14. R²⁵ and R²⁶ Groups

In one aspect, each of R²⁵ and R²⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl.

In a further aspect, R²⁵ is hydrogen. In a further aspect, R²⁵ is selected from trifluoromethyl, carboxamido, alkylsulfonyl, and an optionally substituted C1 to C6 organic residue. In a further aspect, R²⁵ is selected from trifluoromethyl, carboxamido, and alkylsulfonyl. In a further aspect, R²⁵ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R²⁶ is hydrogen. In a further aspect, R²⁶ is selected from trifluoromethyl, carboxamido, alkylsulfonyl, and an optionally substituted C1 to C6 organic residue. In a further aspect, R²⁶ is selected from trifluoromethyl, carboxamido, and alkylsulfonyl. In a further aspect, R²⁶ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R²⁶ is hydrogen and wherein R²⁵ is selected from trifluoromethyl, carboxamido, alkylsulfonyl, and an optionally substituted C1 to C6 organic residue. In a further aspect, R²⁶ is hydrogen and wherein R²⁵ is selected from trifluoromethyl, carboxamido, and alkylsulfonyl. In a further aspect, R²⁶ is hydrogen and wherein R²⁵ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R²⁵ is hydrogen and wherein R²⁶ is selected from trifluoromethyl, carboxamido, thiol, alkylsulfonyl, and an optionally substituted C1 to C6 organic residue. In a further aspect, R²⁵ is hydrogen and wherein R²⁶ is selected from trifluoromethyl, carboxamido, and alkylsulfonyl. In a further aspect, R²⁵ is hydrogen and wherein R²⁶ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R²⁵ and R²⁶, together with the intermediate carbon, comprise an optionally substituted C3 to C6 cycloalkyl. In a further aspect, wherein R²⁵ and R²⁶, together with the intermediate carbon, comprise cyclopropyl, cyclobutyl, cyclopentyl, or cyclohexyl.

15. R²⁷ and R²⁸ Groups

In one aspect, each of R²⁷ and R²⁸ independently comprises hydrogen, trifluoromethyl, carboxamido, alkylsulfonyl, an optionally substituted C1 to C6 alkyl, or an optionally substituted C3 to C6 cycloalkyl or R²⁷ and R²⁸, together with the intermediate carbon, comprise an optionally substituted C3 to C6 cycloalkyl.

In a further aspect, R²⁷ is hydrogen. In a further aspect, R²⁷ is selected from trifluoromethyl, carboxamido, alkylsulfonyl, and an optionally substituted C1 to C6 organic residue. In a further aspect, R²⁷ is selected from trifluoromethyl, carboxamido, and alkylsulfonyl. In a further aspect, R²⁷ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl. In a further aspect, R²⁷ is methyl.

In a further aspect, R²⁸ is hydrogen. In a further aspect, R²⁸ is selected from trifluoromethyl, carboxamido, alkylsulfonyl, and an optionally substituted C1 to C6 organic residue. In a further aspect, R²⁸ is selected from trifluoromethyl, carboxamido, and alkylsulfonyl. In a further aspect, R²⁸ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl. In a further aspect, R²⁸ is methyl.

In a further aspect, R²⁸ is hydrogen and wherein R²⁷ is selected from trifluoromethyl, carboxamido, alkylsulfonyl, and an optionally substituted C1 to C6 organic residue. In a further aspect, R²⁸ is hydrogen and wherein R²⁷ is selected from trifluoromethyl, carboxamido, and alkylsulfonyl. In a further aspect, R²⁸ is hydrogen and wherein R²⁷ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R²⁷ is hydrogen and wherein R²⁸ is selected from trifluoromethyl, carboxamido, alkylsulfonyl, and an optionally substituted C1 to C6 organic residue. In a further aspect, R²⁷ is hydrogen and wherein R²⁸ is selected from trifluoromethyl, carboxamido, and alkylsulfonyl. In a further aspect, R²⁷ is hydrogen and wherein R²⁸ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R²⁷ and R²⁸, together with the intermediate carbon, comprise an optionally substituted C3 to C6 cycloalkyl. In a further aspect, R²⁷ and R²⁸, together with the intermediate carbon, comprise cyclopropyl, cyclobutyl, cyclopentyl, or cyclohexyl.

16. R²⁹ Groups

In one aspect, R²⁹ comprises hydrogen, an optionally substituted C1 to C6 alkyl, an optionally substituted C3 to C6 cycloalkyl, or a hydrolysable residue.

In a further aspect, R²⁹ is hydrogen. In a further aspect, R²⁹ is an optionally substituted C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl. In a further aspect, R²⁹ is an optionally substituted C3 to C6 cycloalkyl selected from cyclopropyl, cyclobutyl, cyclopentyl, and cyclohexyl. In a further aspect, R⁹ is a hydrolysable residue.

17. R³⁰ Groups

In one aspect, R³⁰ comprises an optionally substituted C1 to C12 organic residue selected from alkyl, aryl, heteroaryl, cycloalkyl, heterocycloalkyl, cycloalkenyl, and heterocycloalkenyl.

In a further aspect, R³⁰ is an optionally substituted alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, cyclohexyl, heptyl, cycloheptyl, octyl, cyclooctyl, nonyl, cyclononyl, decyl, cyclodecyl, undecyl, cycloundecyl, dodecyl, or cyclododecyl.

In a further aspect, R³⁰ is an optionally substituted aryl selected from phenyl and naphthyl.

In a further aspect, R³⁰ is an optionally substituted heteroaryl selected from furanyl, pyranyl, imidazolyl, thiophenyl, pyridinyl, pyridazinyl, pyrimidinyl, pyrazinyl, triazinyl, tetrazinyl, benzofuranyl, benzothiophene, indolyl, indazolyl, quinolinyl, naphthyridinyl, benzothiazolyl, benzooxazolyl, benzoimidazolyl, and benzotriazolyl.

In a further aspect, R³⁰ is an optionally substituted cycloalkyl selected from cyclopropyl, cyclobutyl, cyclopentyl, cyclohexyl, cycloheptyl, cyclooctyl, cyclononyl, bicyclo[3.1.0]hexyl, bicyclo[4.1.0]heptyl, bicyclo[5.1.0]octyl, bicyclo[6.1.0]nonyl, bicyclo[3.2.0]heptyl, bicyclo[4.2.0]octyl, bicyclo[5.2.0]nonyl, bicyclo[3.3.0]octyl, bicyclo[4.3.0]nonyl, bicyclo[2.2.1]heptyl, bicyclo[3.2.1]octyl, bicyclo[4.2.1]nonyl, bicyclo[2.2.2]octyl, bicyclo[3.2.2]nonyl, and bicyclo[3.3.1]nonyl.

In a further aspect, R³⁰ is an optionally substituted heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R³⁰ is optionally substituted cycloalkenyl selected from cyclobutenyl, cyclopentenyl, cyclopentadienyl, cyclohexenyl, cyclohexadienyl, cycloheptenyl, cycloheptadienyl, cyclooctenyl, cyclooctadienyl, cyclononenyl, and cyclononadienyl.

In a further aspect, R³⁰ is optionally substituted heterocycloalkenyl comprising a mono-, di- or tri-unsaturated analog of a heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R³⁰ is phenylethynyl, indolyl, quinolinyl, naphthyl, phenylcyclopropyl, or fluorophenyl.

18. R^{43a} and R^{41b} Groups

In one aspect, each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each of R^{41a} and R^{41b} is hydrogen. In a further aspect, each of R^{41a} and R^{41b} is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each of R^{41a} and R^{41b} is independently selected from halide, hydroxyl, trifluoromethyl, amino, cyano, nitro, azide, carboxamido, alkoxy, thiol, and alkylsulfonyl. In a further aspect, at least one of R^{41a} and R^{41b} is methyl, ethyl, n-propyl, propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

19. R^{42a} and R^{42b} Groups

In one aspect, each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each of R^{42a} and R^{42b} is hydrogen. In a further aspect, each of R^{42a} and R^{42b} is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each of R^{42a} and R^{42b} is independently selected from halide, hydroxyl, trifluoromethyl, amino, cyano, nitro, azide, carboxamido, alkoxy, thiol, and alkylsulfonyl. In a further aspect, at least one of R^{42a} and R^{42b} is methyl, ethyl, n-propyl, propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

20. R⁴³ Groups

In one aspect, R⁴³ comprises hydrogen, an optionally substituted C1 to C6 alkyl, an optionally substituted C3 to C6 cycloalkyl, or a hydrolysable residue.

In a further aspect, R⁴³ is hydrogen. In a further aspect, R⁴³ is an optionally substituted C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl. In a further aspect, R⁴³ is an optionally substituted C3 to C6 cycloalkyl selected from cyclopropyl, cyclobutyl, cyclopentyl, cyclohexyl, and bicyclo[3.1.0]hexyl. In a further aspect, R⁴³ is a hydrolysable residue.

21. R⁴⁴ Groups

In one aspect, R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue.

In a further aspect, each R⁴⁴ is hydrogen. In a further aspect, each R⁴⁴ is independently selected from halide, hydroxyl, trifluoromethyl, amino, cyano, nitro, azide, carboxamido, alkoxy, thiol, alkylsulfonyl, and an optionally substituted C1 to C6 organic residue. In a further aspect, each R⁴⁴ is independently selected from halide, hydroxyl, trifluoromethyl, amino, cyano, nitro, azide, carboxamido, alkoxy, thiol, and alkylsulfonyl. In a further aspect, at least one R⁴⁴ is methyl, ethyl, n-propyl, i-propyl, cyclopropyl, n-butyl, butyl, s-butyl, cyclobutyl, n-pentyl, i-pentyl, s-pentyl, neopentyl, cyclopentyl, n-hexyl, hexyl, s-hexyl, dimethylbutyl, or cyclohexyl.

22. R⁴⁵ and R⁴⁶ Groups

In one aspect, each of R⁴⁵ and R⁴⁶ independently comprises hydrogen, trifluoromethyl, carboxamido, alkylsulfonyl, an optionally substituted C1 to C6 alkyl, or an optionally substituted C3 to C6 cycloalkyl or R⁴⁵ and R⁴⁶, together with the intermediate carbon, comprise an optionally substituted C3 to C6 cycloalkyl.

In a further aspect, R⁴⁵ is hydrogen. In a further aspect, R⁴⁵ is selected from trifluoromethyl, carboxamido, alkylsulfonyl, and an optionally substituted C1 to C6 organic residue. In a further aspect, R⁴⁵ is selected from trifluoromethyl, carboxamido, and alkylsulfonyl. In a further aspect, R⁴⁵ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁴⁶ is hydrogen. In a further aspect, R⁴⁶ is selected from trifluoromethyl, alkoxy, thiol, alkylsulfonyl, and an optionally substituted C1 to C6 organic residue. In a further aspect, R⁴⁶ is selected from trifluoromethyl, carboxamido, and alkylsulfonyl. In a further aspect, R⁴⁶ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁴⁶ is hydrogen and wherein R⁴⁵ is selected from trifluoromethyl, carboxamido, alkylsulfonyl, and an optionally substituted C1 to C6 organic residue. In a further aspect, R⁴⁶ is hydrogen and wherein R⁴⁵ is selected from trifluoromethyl, carboxamido, and alkylsulfonyl. In a further aspect, R⁴⁶ is hydrogen and wherein R⁴⁵ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁴⁵ is hydrogen and wherein R⁴⁶ is selected from trifluoromethyl, carboxamido, alkylsulfonyl, and an optionally substituted C1 to C6 organic residue. In a further aspect, R⁴⁵ is hydrogen and wherein R⁴⁶ is selected from trifluoromethyl, carboxamido, and alkylsulfonyl. In a further aspect, R⁴⁵ is hydrogen and wherein R⁴⁶ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁴⁵ and R⁴⁶, together with the intermediate carbon, comprise an optionally substituted C3 to C6 cycloalkyl. In a further aspect, wherein R⁴⁵ and R⁴⁶, together with the intermediate carbon, comprise cyclopropyl, cyclobutyl, cyclopentyl, or cyclohexyl.

23. R⁴⁷ and R⁴⁸ Groups

In one aspect, each of R⁴⁷ and R⁴⁸ independently comprises hydrogen, trifluoromethyl, carboxamido, alkylsulfonyl, an optionally substituted C1 to C6 alkyl, or an optionally substituted C3 to C6 cycloalkyl or R⁴⁷ and R⁴⁸, together with the intermediate carbon, comprise an optionally substituted C3 to C6 cycloalkyl.

In a further aspect, R⁴⁷ is hydrogen. In a further aspect, R⁴⁷ is selected from trifluoromethyl, carboxamido, alkylsulfonyl, and an optionally substituted C1 to C6 organic residue. In a further aspect, R⁴⁷ is selected from trifluoromethyl, carboxamido, and alkylsulfonyl. In a further aspect, R⁴⁷ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl. In a further aspect, R⁴⁷ is methyl.

In a further aspect, R⁴⁸ is hydrogen. In a further aspect, R⁴⁸ is selected from trifluoromethyl, carboxamido, alkylsulfonyl, and an optionally substituted C1 to C6 organic residue. In a further aspect, R⁴⁸ is selected from trifluoromethyl, carboxamido, and alkylsulfonyl. In a further aspect, R⁴⁸ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl. In a further aspect, R⁴⁸ is methyl.

In a further aspect, R⁴⁸ is hydrogen and wherein R⁴⁷ is selected from trifluoromethyl, carboxamido, alkoxy, thiol, alkylsulfonyl, and an optionally substituted C1 to C6 organic residue. In a further aspect, R⁴⁸ is hydrogen and wherein R⁴⁷ is selected from trifluoromethyl, carboxamido, and alkylsulfonyl. In a further aspect, R⁴⁸ is hydrogen and wherein R⁴⁷ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁴⁷ is hydrogen and wherein R⁴⁸ is selected from trifluoromethyl, carboxamido, alkylsulfonyl, and an optionally substituted C1 to C6 organic residue. In a further aspect, R⁴⁷ is hydrogen and wherein R⁴⁸ is selected from trifluoromethyl, carboxamido, and alkylsulfonyl. In a further aspect, R⁴⁷ is hydrogen and wherein R⁴⁸ is methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, or cyclohexyl.

In a further aspect, R⁴⁷ and R⁴⁸, together with the intermediate carbon, comprise an optionally substituted C3 to C6 cycloalkyl. In a further aspect, R⁴⁷ and R⁴⁸, together with the intermediate carbon, comprise cyclopropyl, cyclobutyl, cyclopentyl, or cyclohexyl.

24. R⁴⁹ Groups

In one aspect, R⁴⁹ comprises hydrogen, an optionally substituted C1 to C6 alkyl, an optionally substituted C3 to C6 cycloalkyl, or a hydrolysable residue.

In a further aspect, R⁴⁹ is hydrogen. In a further aspect, R⁴⁹ is an optionally substituted C1 to C6 alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, and cyclohexyl. In a further aspect, R⁴⁹ is an optionally substituted C3 to C6 cycloalkyl selected from cyclopropyl, cyclobutyl, cyclopentyl, and cyclohexyl. In a further aspect, R⁹ is a hydrolysable residue.

25. R⁵⁰ Groups

In one aspect, R⁵⁰ comprises an optionally substituted C1 to C16 organic residue selected from alkyl, aryl, heteroaryl, cycloalkyl, heterocycloalkyl, cycloalkenyl, and heterocycloalkenyl.

In a further aspect, R⁵⁰ is an optionally substituted alkyl selected from methyl, ethyl, n-propyl, i-propyl, cyclopropyl, n-butyl, i-butyl, s-butyl, cyclobutyl, n-pentyl, i-pentyl, s-pentyl, neopentyl, cyclopentyl, n-hexyl, i-hexyl, s-hexyl, dimethylbutyl, cyclohexyl, heptyl, cycloheptyl, octyl, cyclooctyl, nonyl, cyclononyl, decyl, cyclodecyl, undecyl, cycloundecyl, dodecyl, or cyclododecyl.

In a further aspect, R⁵⁰ is an optionally substituted aryl selected from phenyl and naphthyl.

In a further aspect, R⁵⁰ is an optionally substituted heteroaryl selected from furanyl, pyranyl, imidazolyl, thiophenyl, pyridinyl, pyridazinyl, pyrimidinyl, pyrazinyl, triazinyl, tetrazinyl, benzofuranyl, benzothiophene, indolyl, indazolyl, quinolinyl, naphthyridinyl, benzothiazolyl, benzooxazolyl, benzoimidazolyl, and benzotriazolyl.

In a further aspect, R⁵⁰ is an optionally substituted cycloalkyl selected from cyclopropyl, cyclobutyl, cyclopentyl, cyclohexyl, cycloheptyl, cyclooctyl, cyclononyl, bicyclo[3.1.0]hexyl, bicyclo[4.1.0]heptyl, bicyclo[5.1.0]octyl, bicyclo[6.1.0]nonyl, bicyclo[3.2.0]heptyl, bicyclo[4.2.0]octyl, bicyclo[5.2.0]nonyl, bicyclo[3.3.0]octyl, bicyclo[4.3.0]nonyl, bicyclo[2.2.1]heptyl, bicyclo[3.2.1]octyl, bicyclo[4.2.1]nonyl, bicyclo[2.2.2]octyl, bicyclo[3.2.2]nonyl, and bicyclo[3.3.1]nonyl.

In a further aspect, R⁵⁰ is an optionally substituted heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R⁵⁰ is optionally substituted cycloalkenyl selected from cyclobutenyl, cyclopentenyl, cyclopentadienyl, cyclohexenyl, cyclohexadienyl, cycloheptenyl, cycloheptadienyl, cyclooctenyl, cyclooctadienyl, cyclononenyl, and cyclononadienyl.

In a further aspect, R⁵⁰ is optionally substituted heterocycloalkenyl comprising a mono-, di- or tri-unsaturated analog of a heterocycloalkyl selected from oxirane, oxetane, tetrahydrofuran, tetrahydro-2H-pyran, oxepane, oxocane, dioxirane, dioxetane, dioxolane, dioxane, dioxepane, dioxocane, thiirane, thietane, tetrahydrothiophene, tetrahydro-2H-thiopyran, thiepane, thiocane, dithiirane, dithietane, dithiolane, dithiane, dithiepane, dithiocane, oxathiirane, oxathietane, oxathiolane, oxathiane, oxathiepane, oxathiocane, aziridine, azetidine, pyrrolidone, piperidine, azepane, azocane, diaziridine, diazetidine, imidazolidine, piperazine, diazepane, diazocane, hexahydropyrimidine, triazinane, oxaziridine, oxazetidine, oxazolidine, morpholine, oxazepane, oxazocane, thiaziridine, thiazetidine, thiazolidine, thiomorpholine, thiazepane, and thiazocane.

In a further aspect, R⁵⁰ is phenylethynyl, indolyl, quinolinyl, naphthyl, phenylcyclopropyl, or fluorophenyl.

26. Exemplary Compounds

In one aspect, the invention relates to phospholipase D inhibitors comprising one or more compounds selected from:

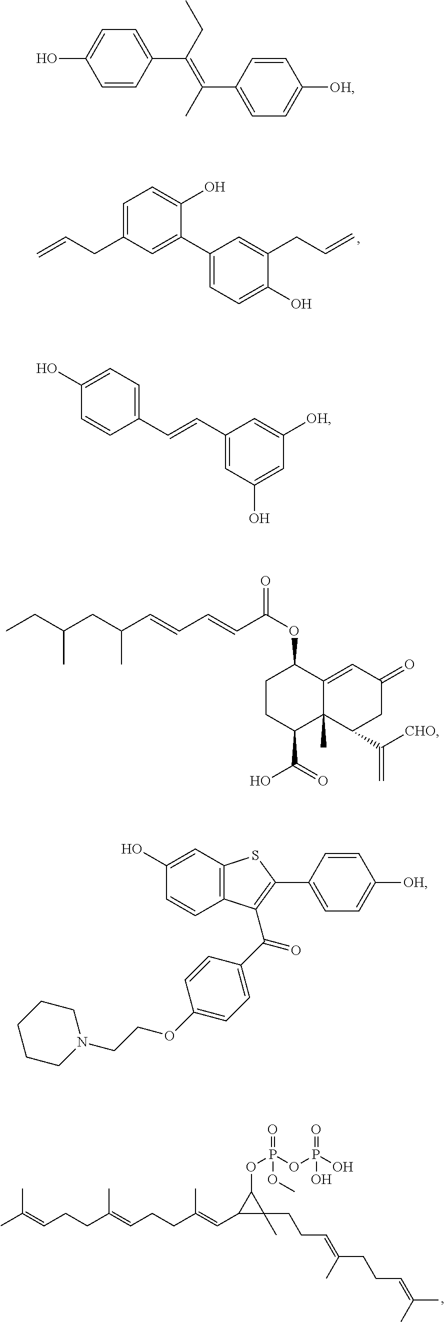

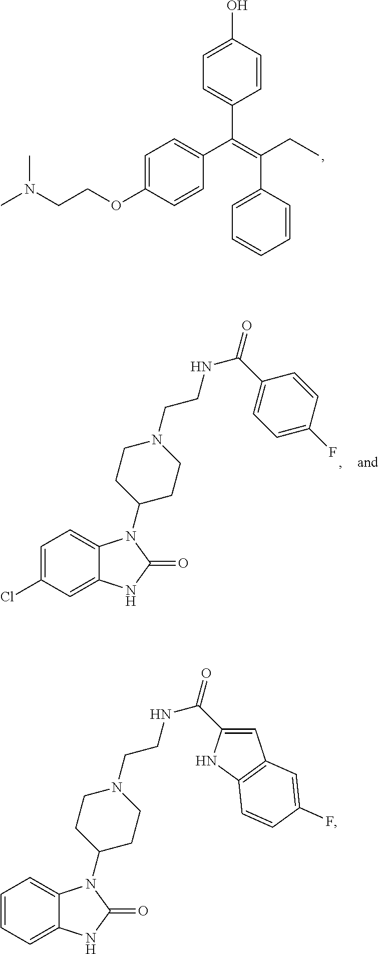 [SMILES: CC/C(=C(\c1ccc(O)cc1)c1ccc(OCCN(C)C)cc1)c1ccccc1 ; O=C(NCCN1CCC(n2c(=O)[nH]c3cc(Cl)ccc32)CC1)c1ccc(F)cc1 ; O=C(NCCN1CCC(n2c(=O)[nH]c3ccccc32)CC1)c1cc2cc(F)ccc2[nH]1]

or a subgroup thereof.

In a further aspect, the invention relates to phospholipase D inhibitors comprising a compound selected from trans-diethylstilbestrol ((E)-4,4′-(hex-3-ene-3,4-diyl)diphenol); resveratrol (5-[2-(4-hydroxyphenyl)ethenyl]benzene-1,3-diol); honokiol (3′,5-diallyl-[1,1′-biphenyl]-2,4′-diol); SCH420789 ((1S,4R,8S,8aR)-4-(((2E,4E)-6,8-dimethyldeca-2,4-dienoyl)oxy)-8a-methyl-6-oxo-8-(3-oxoprop-1-en-2-yl)-1,2,3,4,6,7,8,8a-octahydronaphthalene-1-carboxylic acid); presqualene diphosphate ([[2-(4,8-dimethylnona-3,7-dienyl)-2-methyl-3-(2,6,10-trimethylundeca-1,5,9-trienyl)cyclopropyl]methoxy-hydroxy-phosphoryl]oxyphosphonic acid); raloxifene ((6-hydroxy-2-(4-hydroxyphenyl)benzo[b]thiophen-3-yl)(4-(2-(piperidin-1-yl)ethoxy)phenyl)methanone); 4-hydroxytamoxifen (4-[(Z)-1-[4-[2-(dimethylamino)ethoxy]phenyl]-2-phenylbut-1-enyl]phenol); 5-fluoro-2-indoyl des-chlorohalopemide (N-[2-[4-(2,3-dihydro-2-oxo-1H-benzimidazol-1-yl)-1-piperidinyl]ethyl]-5-fluoro-1H-indole-2-carboxamide), and halopemide (N-[2-[4-(5-Chloro-2,3-dihydro-2-oxo-1H-benzimidazol-1-yl)piperidino]ethyl]-4-fluorobenzamide).

In various aspects, a phospholipase D inhibitor compound can be present as:

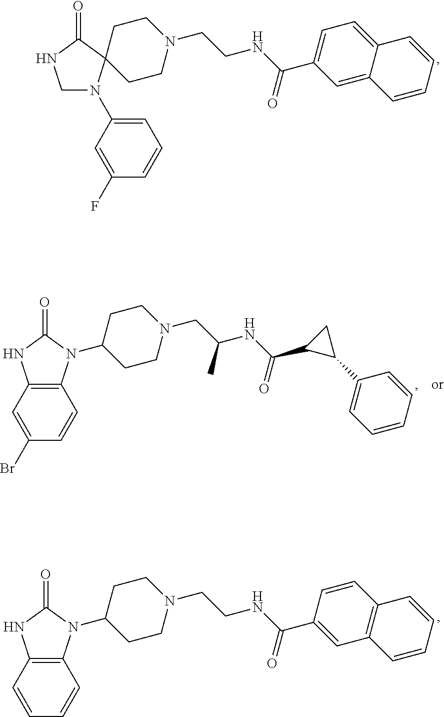

or a subgroup thereof.

In various aspects, a phospholipase D inhibitor compound can be present as:

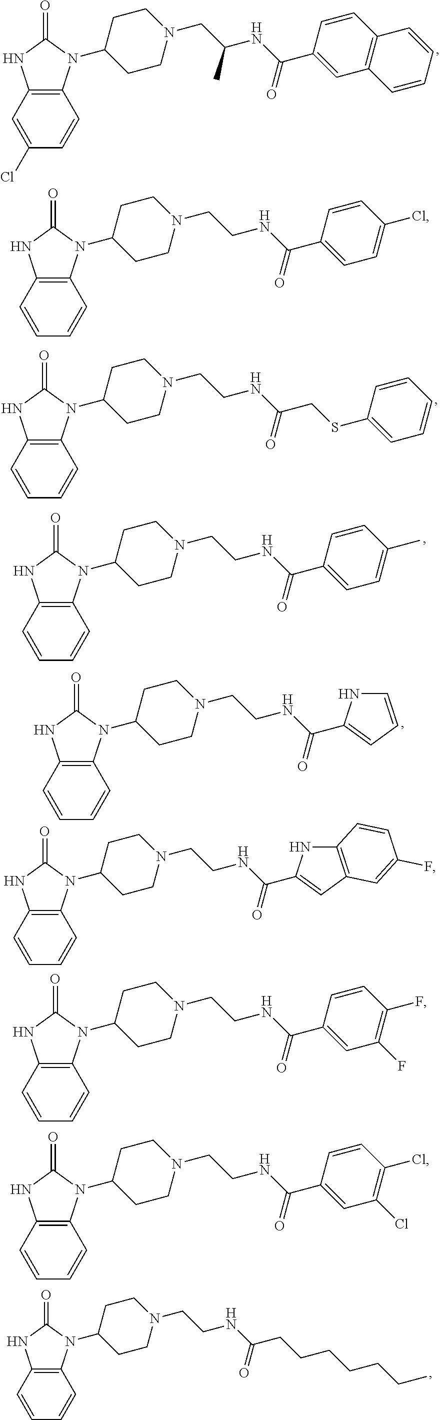

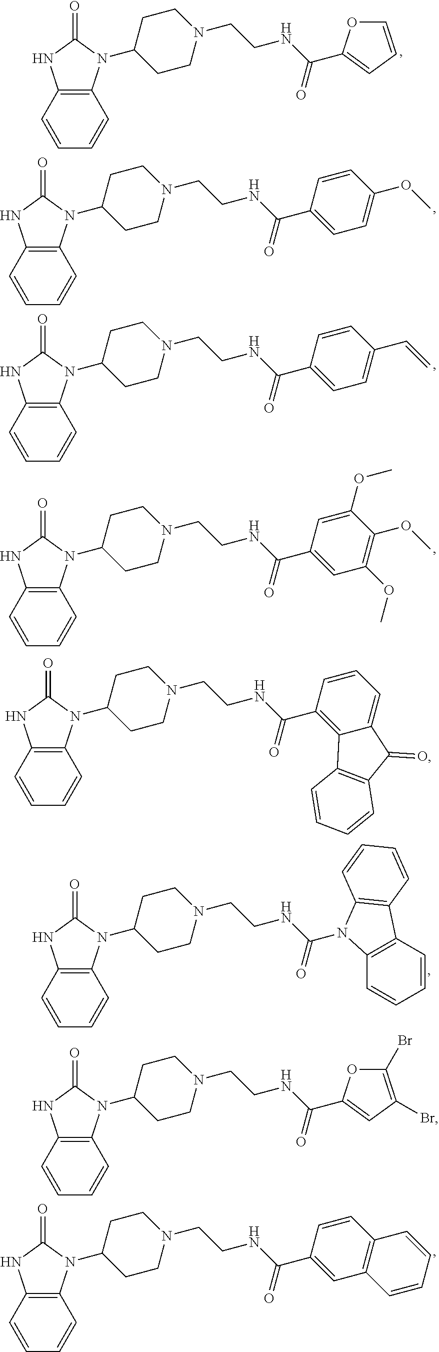

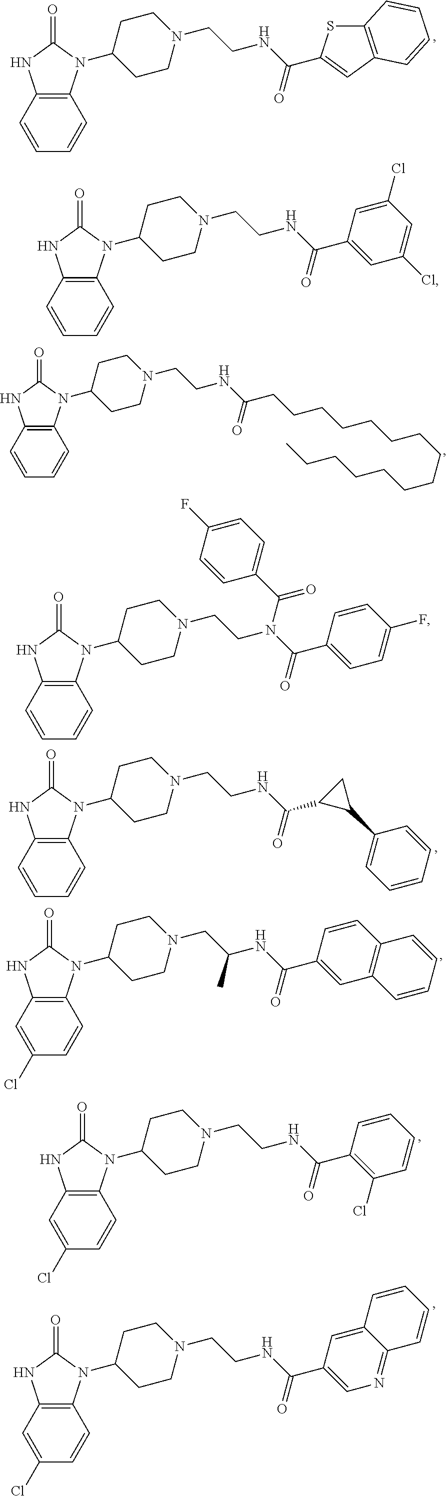

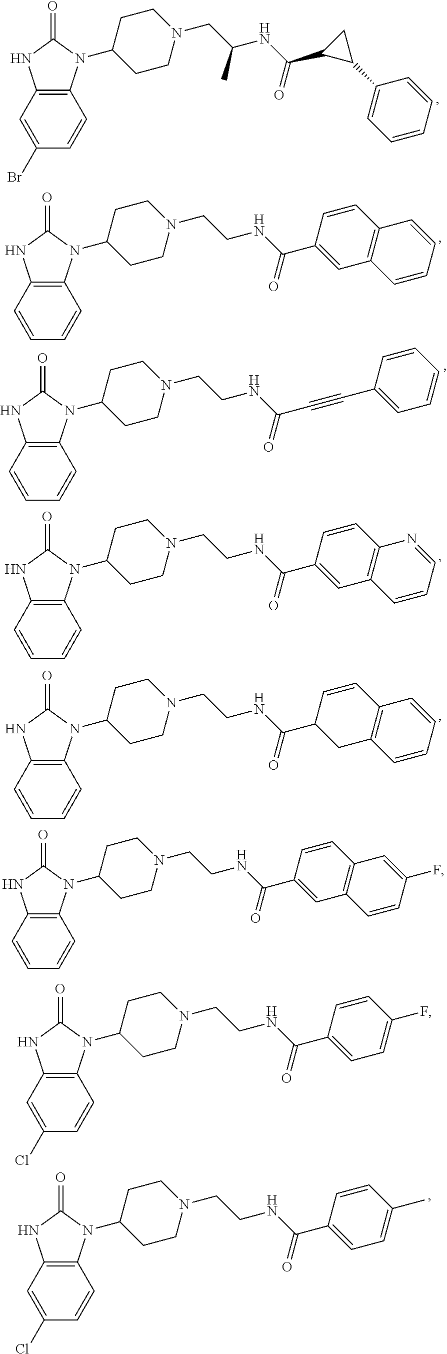

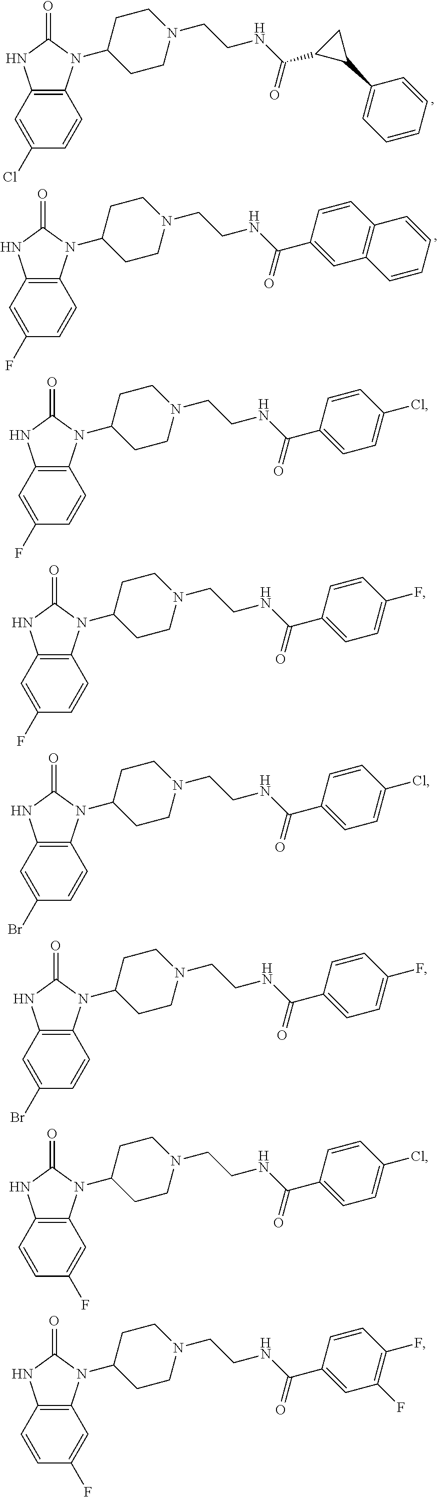

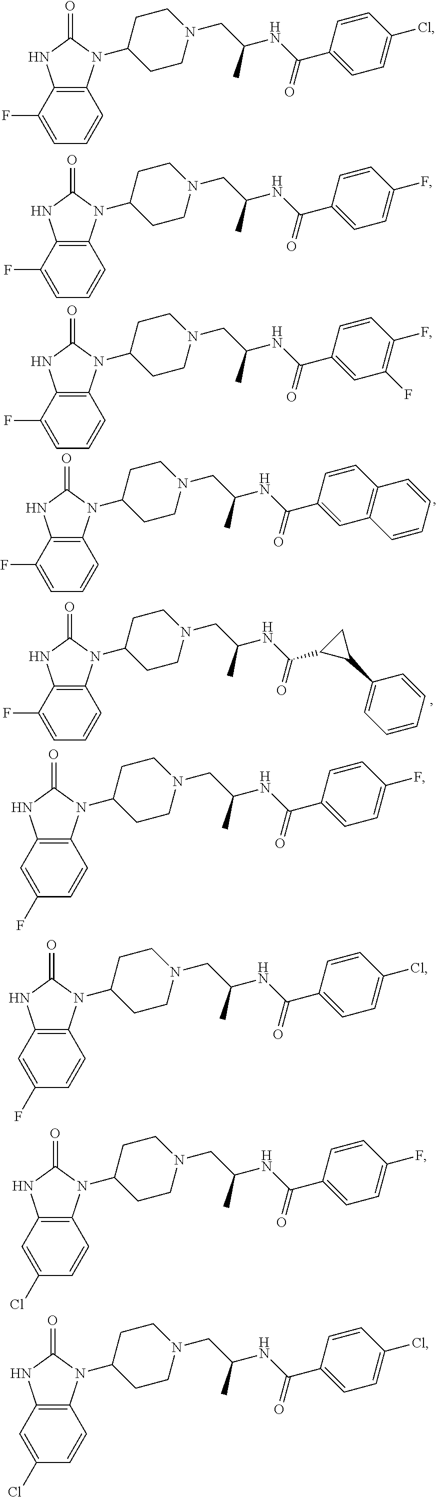

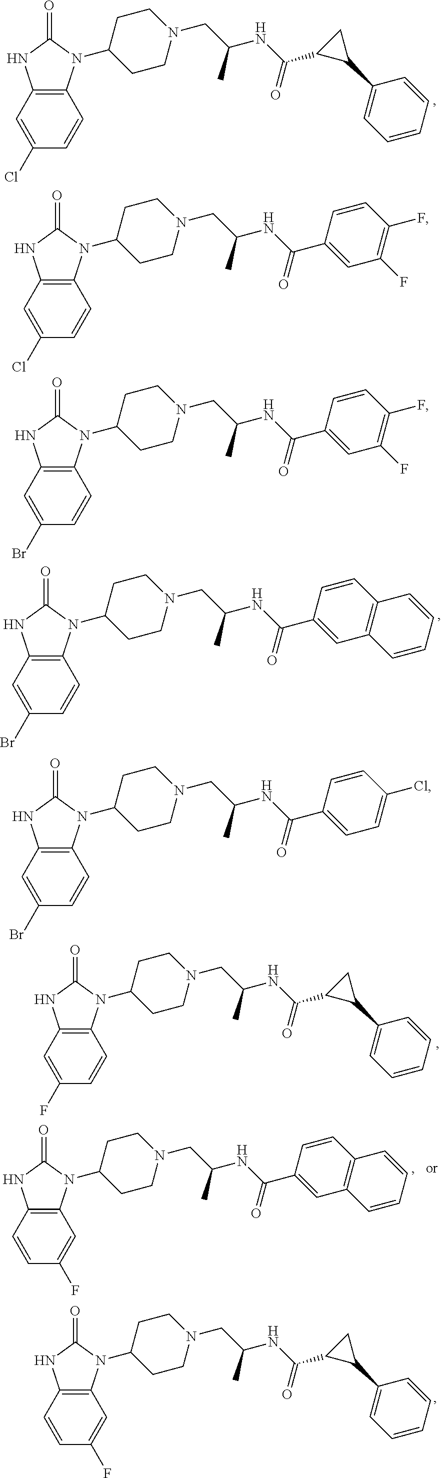

a subgroup thereof.

In various aspects, a phospholipase D inhibitor compound can be present as:

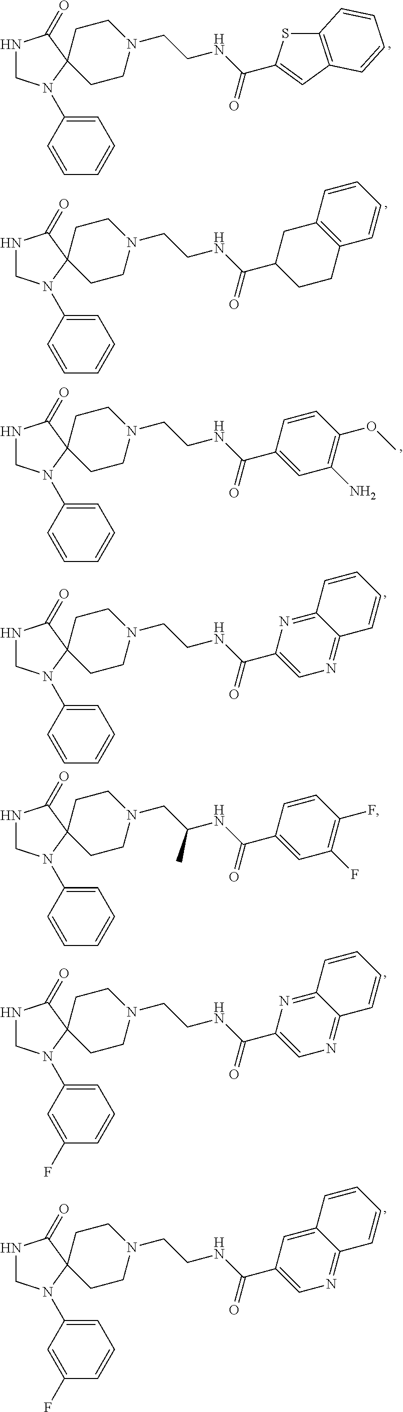

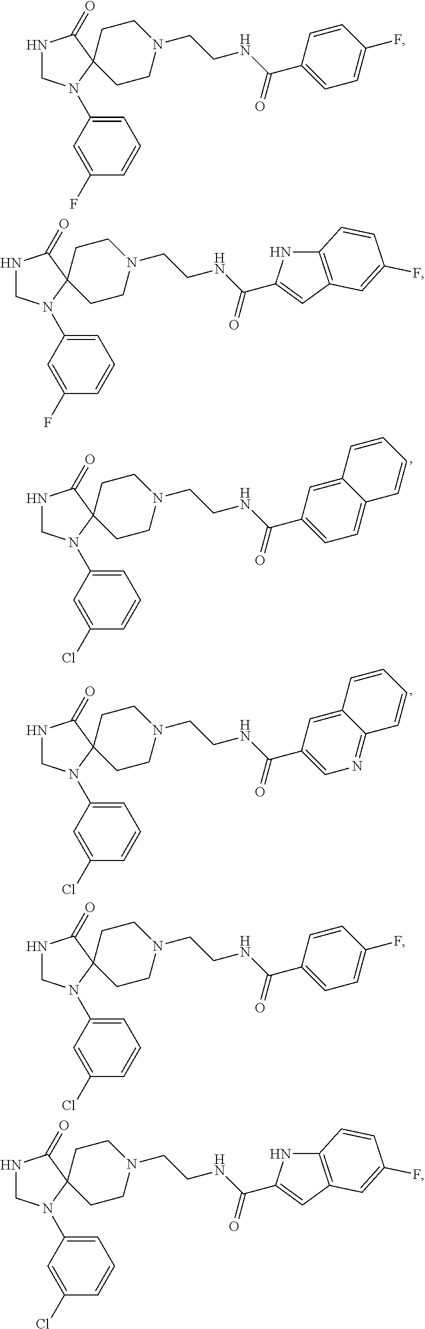

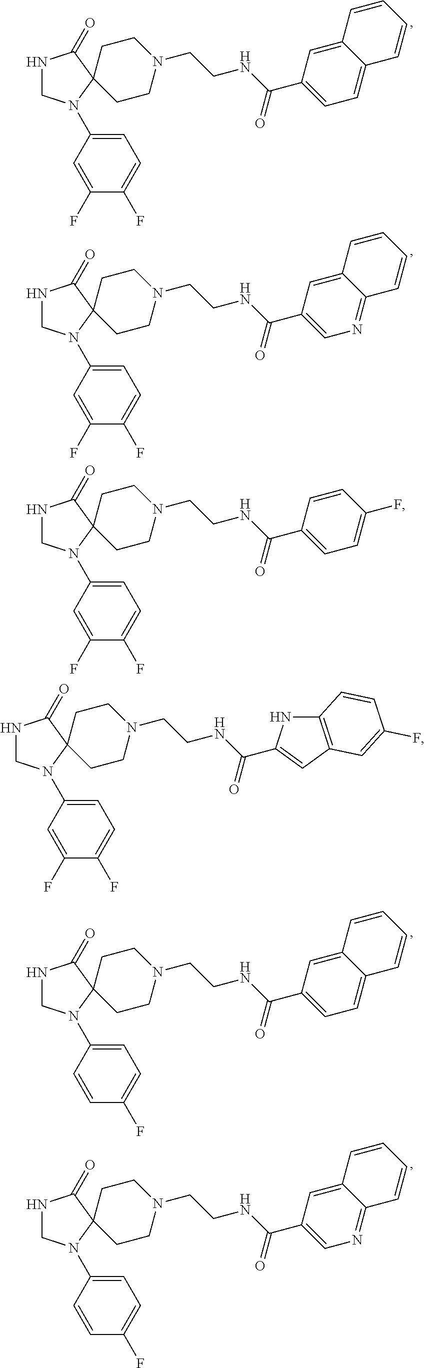

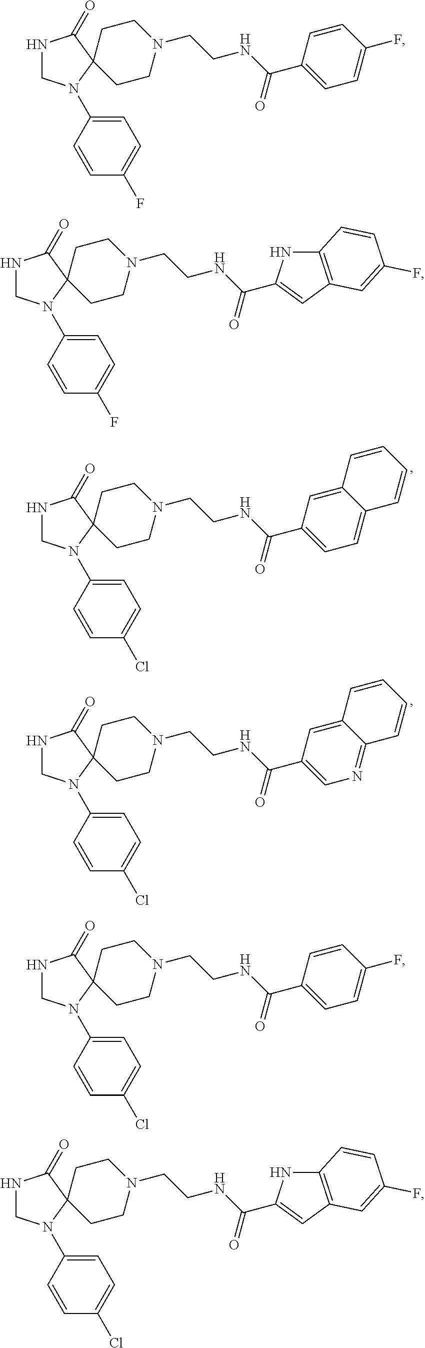

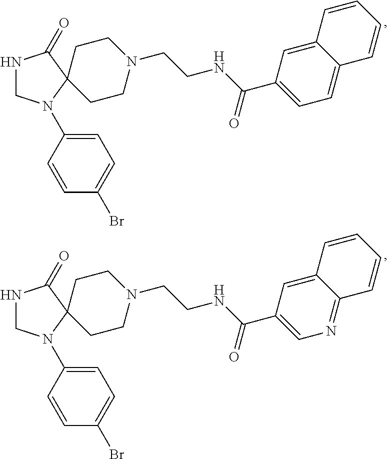 [SMILES: O=C(NCCN1CCC2(CC1)C(=O)NCN2c1ccc(Br)cc1)c1ccc2ccccc2c1 ; O=C(NCCN1CCC2(CC1)C(=O)NCN2c1ccc(Br)cc1)c1cnc2ccccc2c1]

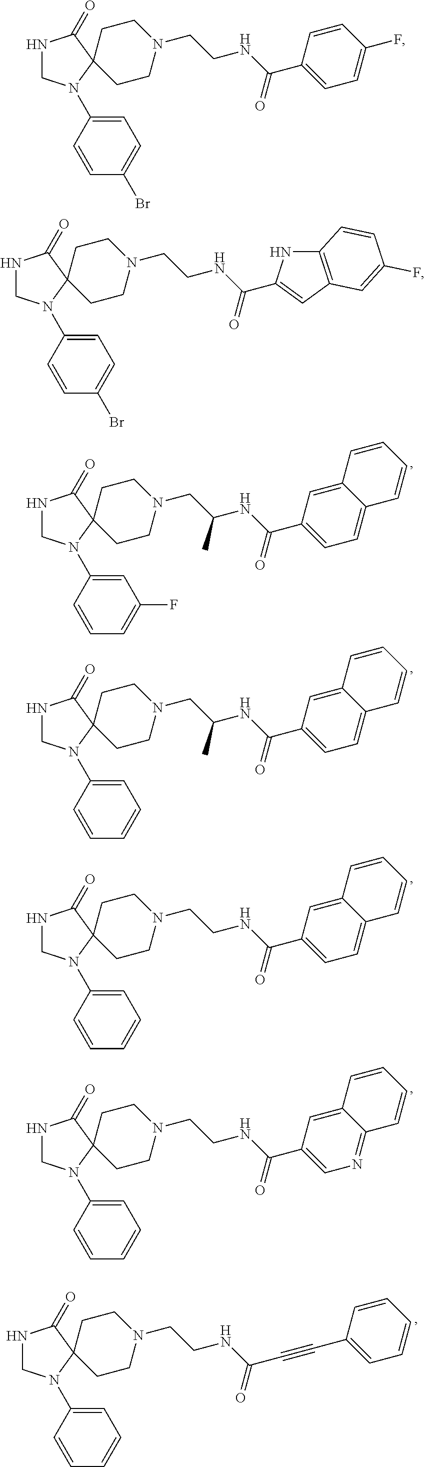

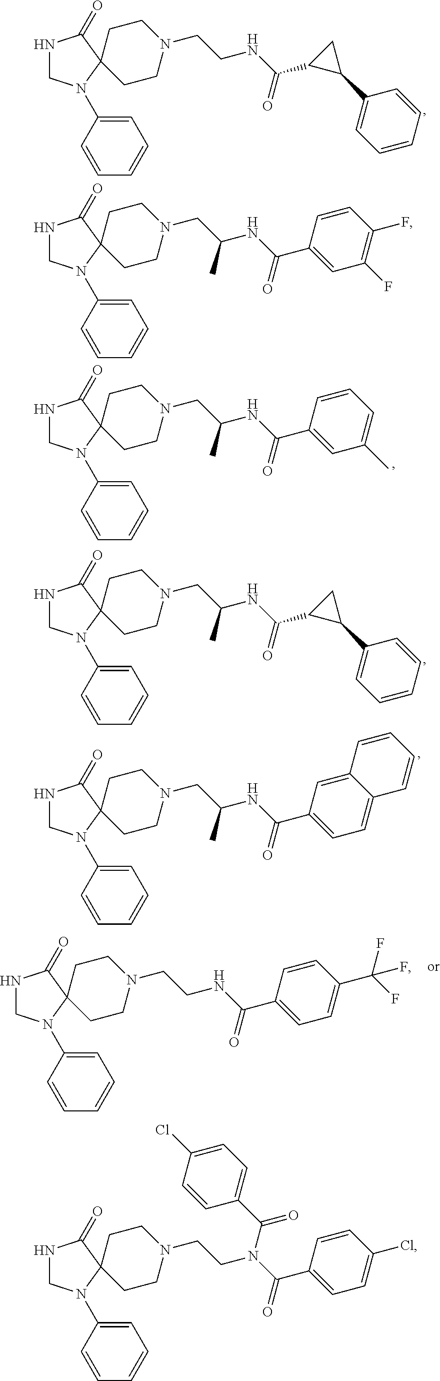

or a subgroup thereof.

In various aspects, a phospholipase D inhibitor compound can be present as:

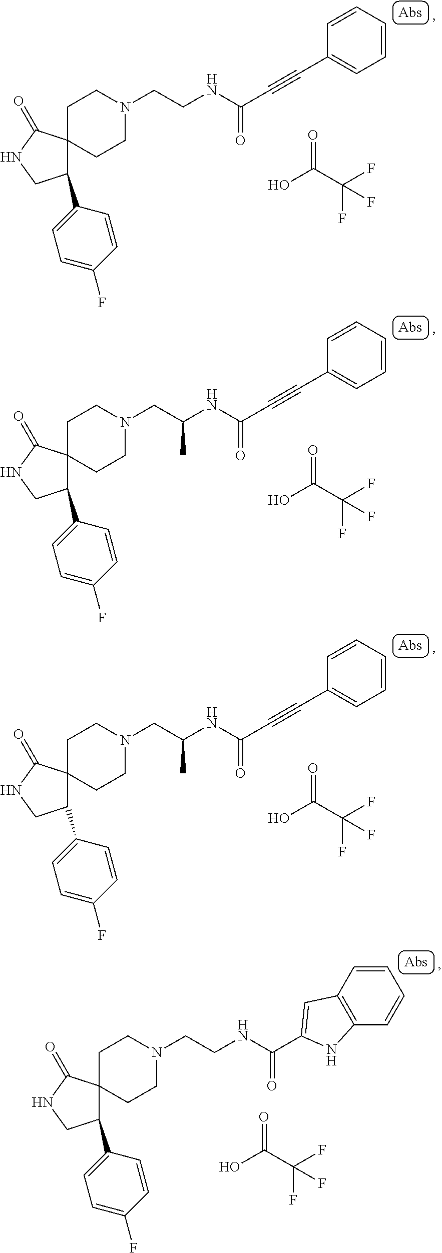

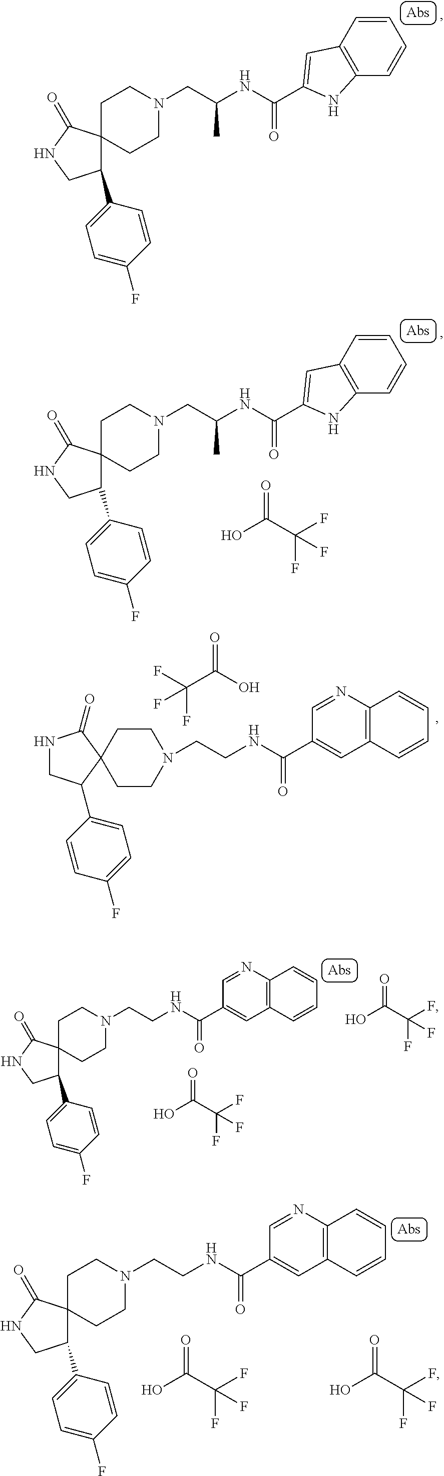

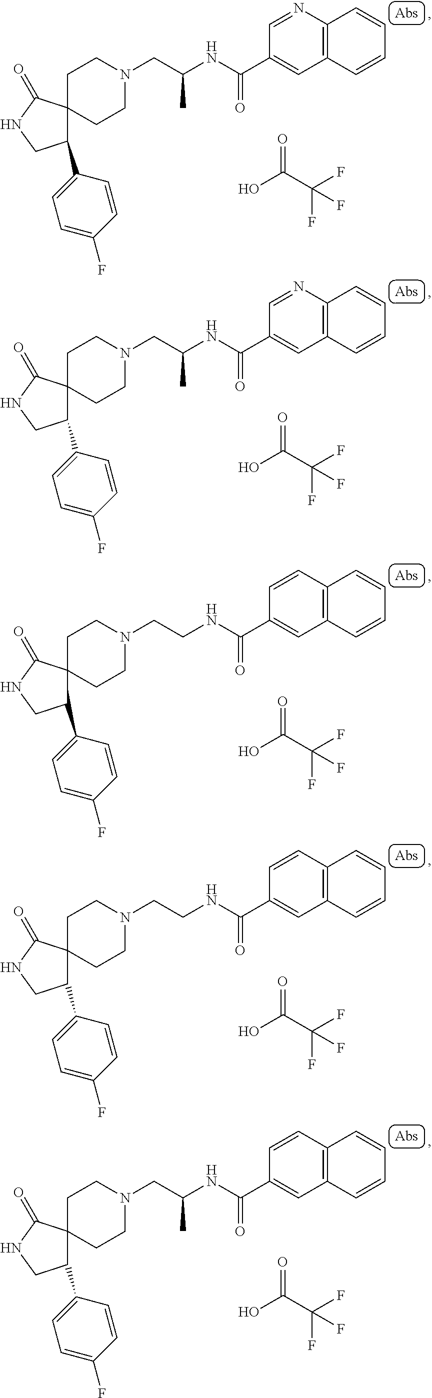

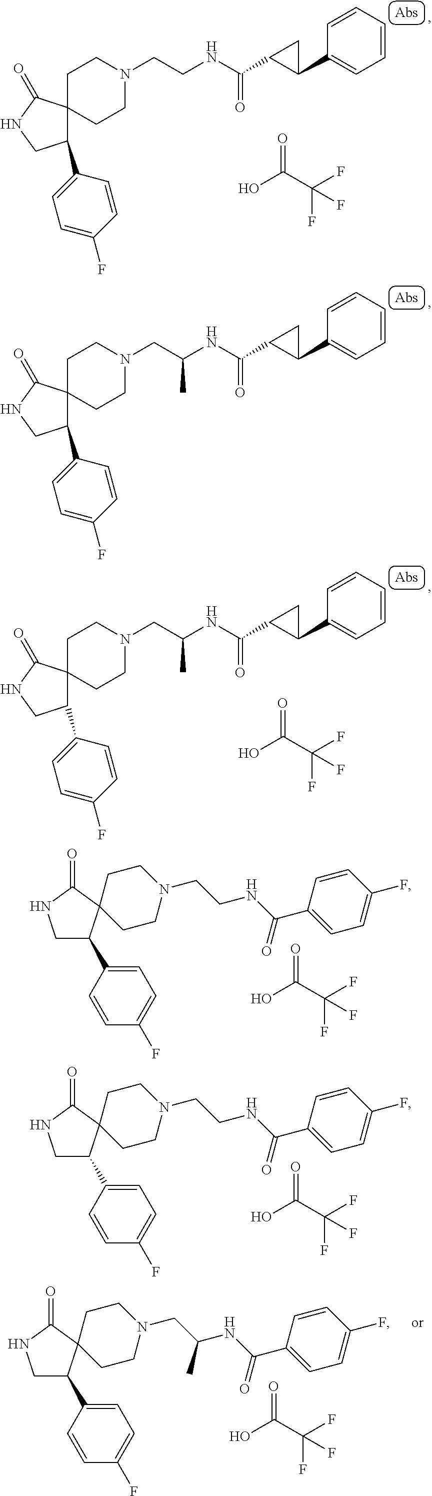

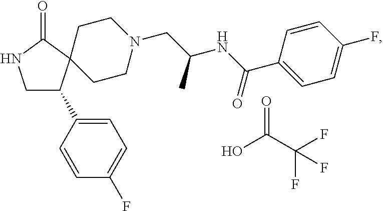

or a subgroup thereof.

### C. PHOSPHOLIPASE D INHIBITION ACTIVITY

In a further aspect, the invention relates to compounds that inhibit a phospholipase D selected from PLD1 and PLD2. In a still further aspect, the compounds inhibit PLD1. In a yet further aspect, the compounds inhibit PLD2. In an even further aspect, the compounds inhibit one or more PLD1 proteins selected from PLD1A, PLD1B, PLD1C, and PLD1D. In a yet further aspect, the compounds inhibit one or more PLD2 selected from PLD2A, PLD2B, and PLD2C.

In one aspect, the compound inhibits PLD activity, i.e. a compound can inhibit PLD1 activity and/or PLD2 activity. In a further aspect, the compound inhibits PLD1 response in an in vitro assay comprising a cultured cell-line. In a further aspect, the compound inhibits PLD1 response in Calu-1 cells. In a further aspect, the compound inhibits PLD2 response in HEK293gfpPLD2 cells. In a further aspect, the compound inhibits in vitro PLD1 response. In a further aspect, the compound inhibits in vitro PLD2 response. For example, the compound can have a PLD1 IC₅₀ of less than about 10 μM, of less than about 5 μM, of less than about 1 μM, of less than about 500 nM, of less than about 100 nM, or of less than about 50 nM. As further examples, the compound can have a PLD2 IC₅₀ of less than about 10 μM, of less than about 5 μM, of less than about 1 μM, of less than about 500 nM, of less than about 100 nM, or of less than about 50 nM.

In a further aspect, the compound can have a PLD1 IC₅₀ of less than about 10 μM, of less than about 1 μM, of less than about 500 nM, of less than about 100 nM, of less than about 60 nM, or of less than about 20 nM. In a further aspect, the compound can have a PLD2 IC₅₀ of less than about 10 μM, of less than about 1 μM, of less than about 500 nM, of less than about 100 nM, of less than about 60 nM, or of less than about 20 nM.

### D. METHODS OF MAKING THE COMPOUNDS

The compounds of this invention can be prepared by employing reactions as shown in the disclosed schemes below, in addition to other standard manipulations that are known in the literature, exemplified in the experimental sections or clear to one skilled in the art. For clarity, examples having a fewer substituent can be shown where multiple substituents are allowed under the definitions disclosed herein. The compounds of this invention can be prepared by employing reactions as disclosed in the references cited herein. For example, suitable methods for synthesizing the disclosed compounds are provided in WO/2011/011680; Scott, S., et al. (2009) Nat. Chem. Biol. 5(2):108-117; Lewis, J. A., et al. (2009) Bioorg. Med. Chem. 19:1916-1920; Lavieri, R., et al. (2009) Bioorg. Med. Chem. 19:2240-2243; and Lavieri, R. R., et al. (2010) J. Med. Chem. 53:6706-6719.

1. Route I

In one aspect, substituted 1-oxo-2,8-diazaspiro[4.5]decanyl analogs of the present invention can be prepared generically by the synthetic scheme as shown below.

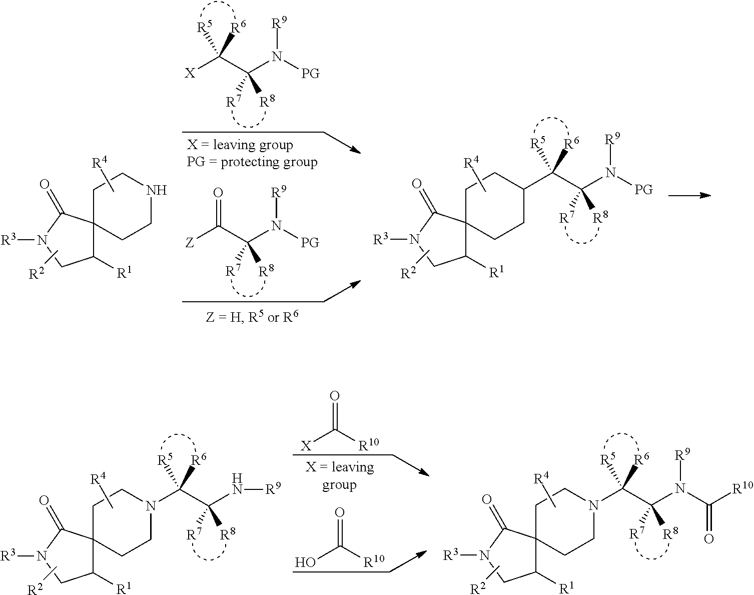

Compounds are represented in generic form, with substituents as noted in compound descriptions elsewhere herein. A more specific example is set forth below.

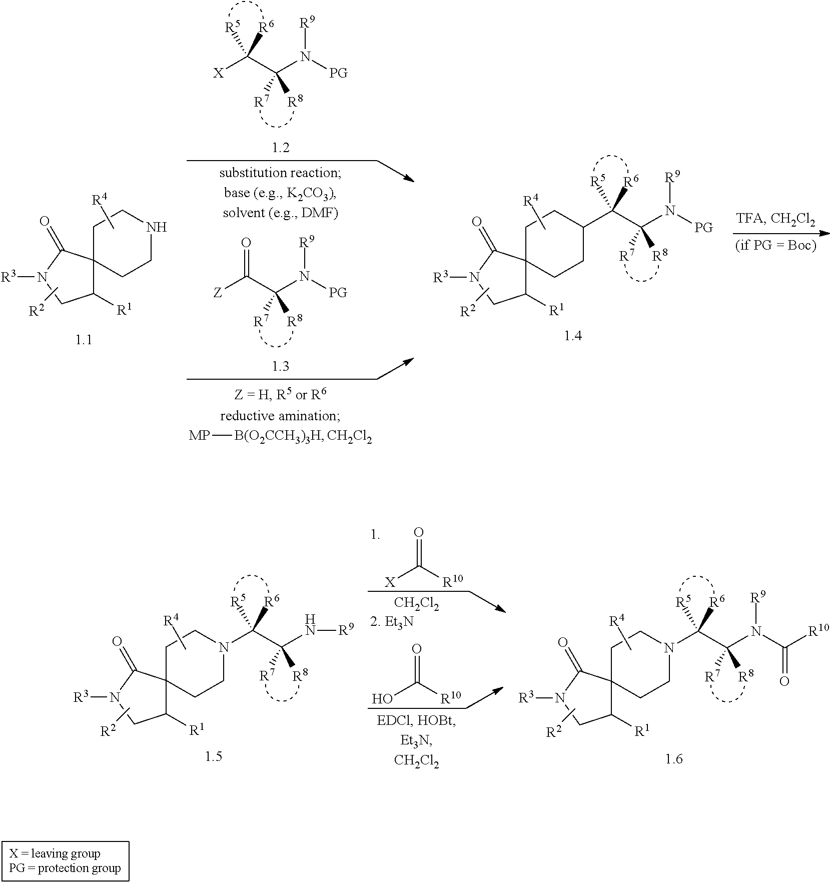

In one aspect, Route I begins with a suitable substituted 2,8-diazaspiro[4.5]decan-1-one (1.1). A suitable 2,8-diazaspiro[4.5]decan-1-one (1.1) is commercially available or can be readily prepared by one skilled in the art. The first reaction of 1.1 and a suitable substituted N-protected amino derivative (1.2) involves a nucleophilic substitution reaction resulting in a N-protected product (1.4). Alternately, the reaction of 1.1 and compound 1.3 [e.g., where R⁵ or R⁶=H, alkyl group, or aryl group] is a reductive amination reaction resulting in a N-protected product (1.4).

In one aspect, the reaction of 1.1 and 1.2 is typically carried out under a suitable reaction atmosphere and in a suitable solvent that supports substitution reactions such as DMF in the presence of an appropriate base such as K₂CO₃. The reaction is conducted at a suitable temperature and for a time sufficient to complete the reaction and to provide compounds of type 1.4 as shown above. The product, a compound of type 1.4, is isolated by methods known to one skilled in the art (e.g., extraction, washing, drying, and concentration under a vacuum; followed by purification, e.g., chromatography, if necessary).

In one aspect, the reaction of 1.1 and 1.3 is typically carried out under a suitable reaction condition that supports reductive amination of carbonyl compounds known to one skilled in the art to give products of type 1.4. Reaction components 1.1 and 1.3 are dissolved in a suitable solvent, e.g., dichloromethane, and stirred at ambient temperature (about 15-30° C.) for about 15 min. Then, the reducing agent, [e.g., macroporous polystyrene triacetoxyborohydride, MP-B(O₂CCH₃)₃H. or other suitable reducing agent] is added to the reaction mixture. The reaction is carried out for a time sufficient to complete the reaction, e.g., overnight (about 8-18 h), to provide compounds of type 1.4 as shown above. The product, a compound of type 1.4, is isolated by methods known to one skilled in the art (e.g., filtered, and concentration under a vacuum; followed by purification, e.g., chromatography, if necessary).

In one aspect, compounds of type 1.5 can be prepared by the conversion of the N-protected compound (e.g., N-Boc compound type 1.4) to the corresponding amine derivative (1.5). For example, a reaction of this type is commonly carried out by dissolving the N-Boc derivative (1.4) in a suitable solvent, e.g., CH₂Cl₂, and then TFA is added. The mixture is stirred for a time sufficient, e.g., about overnight (8-18 h), at ambient room temperature (about 15-30° C.) to complete the reaction. The product (1.8) is isolated by methods known to one skilled in the art (e.g., concentration under a vacuum; followed by purification, e.g., chromatography, if necessary).

In one aspect, compounds of type 1.6 can be prepared by the acylation of 1.5 with an appropriate acid halide of type R¹⁰C(O)X under a standard amine acylation procedure known to one skilled in the art. In an example, R¹⁰C(O)X and the appropriate amine of type 1.5, dissolved in a suitable solvent such as dichloromethane, then an appropriate base, e.g., triethylamine, is added. The reaction is stirred at an appropriate temperature (about 0-30° C.) for about 24-36 h. The product (1.6) is isolated by methods known to one skilled in the art (e.g., concentration under a vacuum; followed by purification, e.g., chromatography, if necessary).

In one aspect, compounds of type 1.6 can be prepared by the acylation of 1.5 with an appropriate carboxylic acid of type R¹⁰CO₂H under a standard carboxylic acid and amine coupling procedure known to one skilled in the art. In an example, R¹⁰ CO₂H, EDCI, HOBt, triethylamine are dissolved in a suitable solvent such as dichloromethane, and allowed to stir for a period of time, e.g., about 15 min. Then, a solution of 1.5, in a solvent, e.g., dichloromethane, is added to the reaction mixture, and the reaction is stirred at ambient temperature (about 15-30° C.) for about 24-36 h. The product (1.6) is isolated by methods known to one skilled in the art (e.g., concentration under a vacuum; followed by purification, e.g., chromatography, if necessary).

2. Route II

In one aspect, substituted 4-oxo-1,3,8-triazaspiro[4.5]decanyl analogs of the present invention can be prepared generically by the synthetic scheme as shown below.

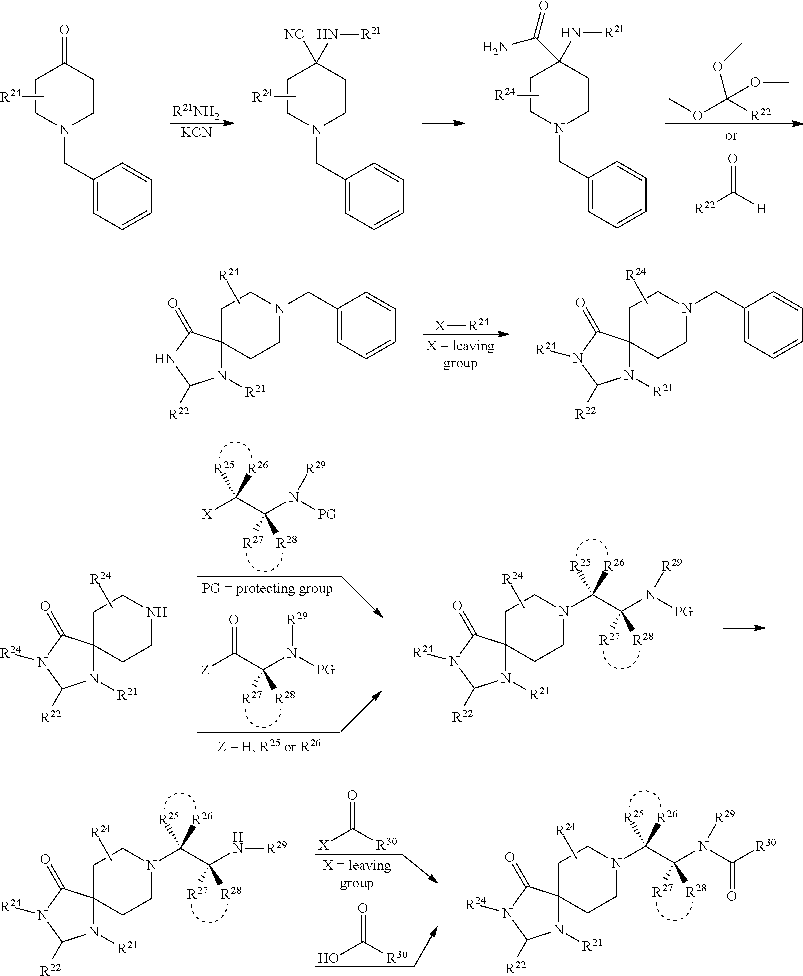

Compounds are represented in generic form, with substituents as noted in compound descriptions elsewhere herein. A more specific example is set forth below.

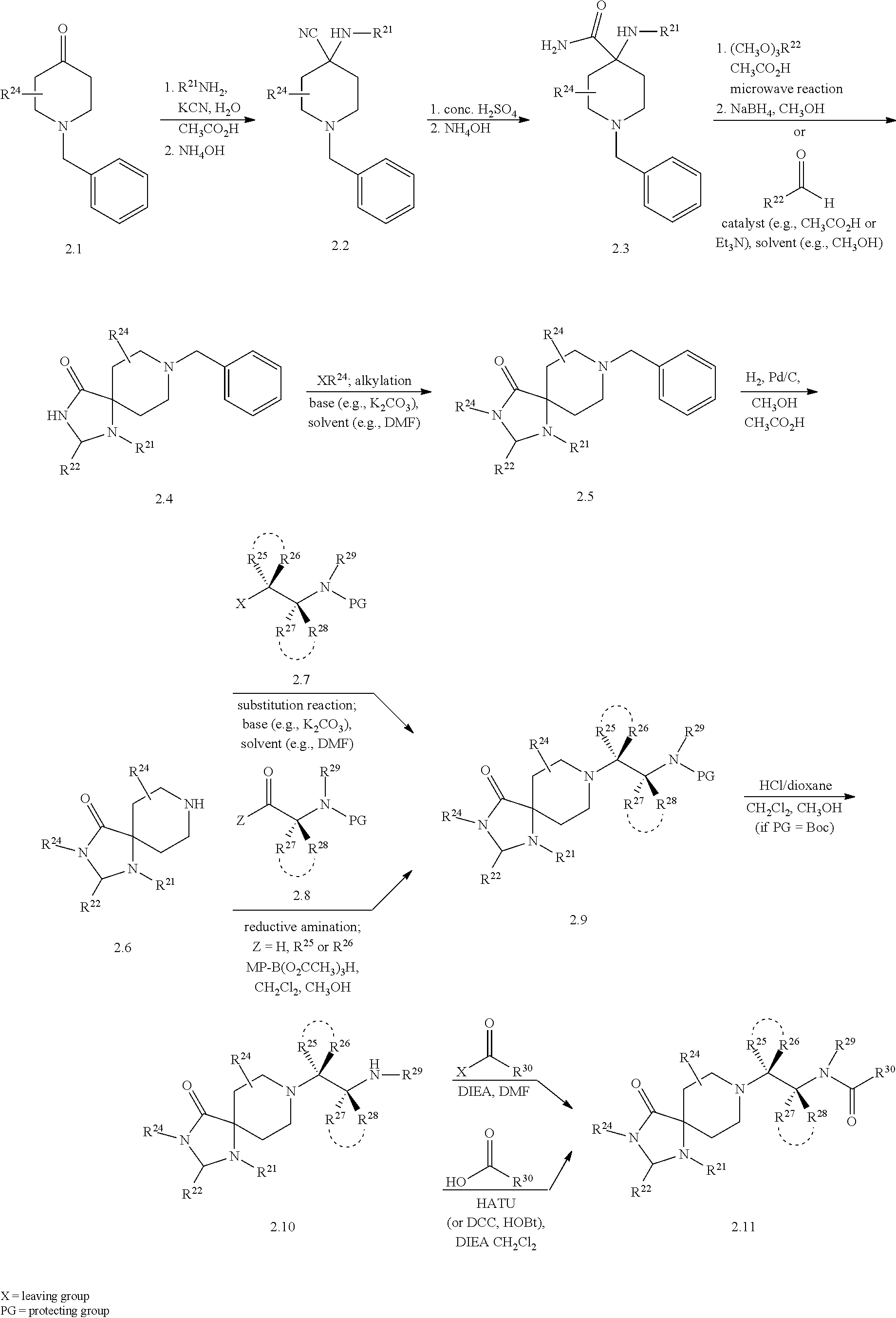

In one aspect, Route II begins with a suitable substituted 1-benzylpiperidine-4-one. A suitable 1-benzylpiperidine-4-one derivatives (2.1) are commercially available or can be readily prepared by one skilled in the art. To a solution of 2.1 in acetic acid and water at about 0° C. is added the amine, R²¹NH₂, and potassium cyanide. The reaction is allowed to warm to about ambient temperature (about 15-30° C.) and agitated/stirred for sufficient time to allow complete reaction to occur (e.g., about 12 h). The reaction is mixture is cooled to about 0° C. and concentrated ammonium hydroxide is added until about pH≧11 is reached. The product (2.2) is isolated by methods known to one skilled in the art (e.g., extraction, and concentration under a vacuum). Immediately following, the unpurified 2.2 is cooled to about 0° C. and concentrated sulfuric acid is added slowly. The reaction is allowed to warm to ambient temperature (about 15-30° C.) with stirring for about 12 h. The reaction is mixture is cooled to about 0° C. and concentrated ammonium hydroxide is added until about pH≧11 is reached. The product (2.3) is isolated by methods known to one skilled in the art (e.g., extraction, and concentration under a vacuum, followed by purification, e.g., chromatography, if necessary).

In one aspect, compounds of type 2.4 can be prepared by the reaction of an appropriate orthoformate derivative [e.g., (CH₃O)₃R²²] and 2.3. Compound 2.3, (CH₃O)₃R²², and acetic acid are combined and subjected to microwave irradiation at an appropriate temperature to effect reaction, e.g., about 150° C., for about 15 min or sufficient time to complete the reaction. Then ammonium hydroxide is added until about pH=12 and extracted with dichloromethane and concentrated under vacuum. The resulting material is added to a suspension of sodium borohydride in methanol and stirred for about 3 h or sufficient time to complete the reaction The reaction is quenched with water. The product (2.4) is isolated by methods known to one skilled in the art (e.g., extraction, and concentration under a vacuum, followed by purification, e.g., chromatography, if necessary).

In one aspect, compounds of type 2.4 can be prepared by the reaction of an appropriate aldehyde (R²²CHO) under in the presence of a suitable acid (e.g., acetic acid) or base (e.g., triethylamine) catalyst in a suitable solvent (e.g., methanol) at suitable reaction temperature and sufficient time to complete the reaction. The product (2.4) is isolated by methods known to one skilled in the art (e.g., extraction, washing, drying, filtering, and concentration under a vacuum, followed by purification, e.g., chromatography, if necessary).

In one aspect, compounds of type 2.5 can be prepared from 2.4 (where R²⁴=H) by alkylation with an appropriate alkyl halide (or similar XR²⁴ where X is an appropriate leaving group or other electrophile to afford the substituent, R²⁴). Compound 2.4 is reacted with an appropriate base (e.g., K₂CO₃) in an appropriate solvent (e.g., DMF) at a sufficient reaction temperature and for sufficient time to allow for complete reaction to afford a product (2.5). The product (2.5) is isolated by methods known to one skilled in the art (e.g., extraction, washing, drying, filtering, and concentration under a vacuum, followed by purification, e.g., chromatography, if necessary).

In one aspect, compounds of type 2.6 can be prepared from 2.5 by hydrogenation. Compound 2.5 is dissolved in a appropriate solvent(s) (e.g., methanol, acetic acid) and treated with an appropriate metal catalyst (e.g., Pd/C) under an atmosphere of hydrogen gas. The reaction is allowed to stir at an appropriate temperature and sufficient time (e.g., about 36 h) to allow for complete reaction to occur. The product (2.6) is isolated by methods known to one skilled in the art (e.g., filtering, adjusting the pH, washing, extraction, drying, filtering, and concentration under a vacuum, followed by purification, e.g., chromatography, if necessary).

In one aspect, the reaction of 2.6 and 2.7 is typically carried out under a suitable reaction atmosphere and in a suitable solvent that supports substitution reactions such as DMF in the presence of an appropriate base such as K₂CO₃. The reaction is conducted at a suitable temperature and for a time sufficient to complete the reaction, to provide compounds of type 2.9 as shown above. The product, a compound of type 2.9, is isolated by methods known to one skilled in the art (e.g., extraction, washing, drying, and concentration under a vacuum; followed by purification, e.g., chromatography, if necessary).

In one aspect, the reaction of 2.6 and 2.8 is typically carried out under a suitable reaction condition that supports reductive amination of carbonyl compounds known to one skilled in the art to give products of type 2.9. Reaction components 2.6 and 2.8 are dissolved in a suitable solvent, e.g., dichloromethane and stirred at ambient temperature (about 15 to 30° C.) for about 15 min. Then, the reducing agent, [e.g., macroporous polystyrene triacetoxyborohydride, MP-B(O₂CCH₃)₃H. or other suitable reducing agent] is added to the reaction mixture. The reaction is carried out for a time sufficient to complete the reaction, e.g., overnight (about 8-18 h), to provide compounds of type 2.9 as shown above. The product, a compound of type 2.9, is isolated by methods known to one skilled in the art (e.g., filtered, and concentration under a vacuum; followed by purification, e.g., chromatography, if necessary).

In one aspect, compounds of type 2.10 can be prepared by the conversion of the N-protected compound (e.g., N-Boc compound type 2.9) to the corresponding amine derivative (2.10). For example, a reaction of this type is commonly carried out by dissolving the N-Boc derivative (2.9) in a suitable solvent(s) (e.g., CH₂Cl₂, CH₃OH) and then HCl (e.g., 4 M HCl in dioxane) is added. The mixture is stirred for a time sufficient, e.g., about 36 h, at ambient room temperature (about 15 to 30° C.) to complete the reaction. The product (2.10) is isolated by methods known to one skilled in the art (e.g., concentration under a vacuum; followed by purification, e.g., chromatography, if necessary).

In one aspect, compounds of type 2.11 can be prepared by the acylation of 2.10 with an appropriate acid halide of type R³⁰C(O)X under a standard amine acylation procedure known to one skilled in the art. In an example, R³⁰C(O)X and the appropriate amine of type 2.10, dissolved in a suitable solvent such as DMF, then an appropriate base, e.g., N,N-diisopropylamine (DIEA), is added at an appropriate temperature (about 0° C.). The mixture is allowed to stir for about 12 h or sufficient time to complete the reaction while slowly warming to ambient temperature (about 15-30° C.). The product (2.11) is isolated by methods known to one skilled in the art (e.g., concentration under a vacuum; followed by purification, e.g., chromatography, if necessary).

In one aspect, compounds of type 2.11 can be prepared by the acylation of 2.10 with an appropriate carboxylic acid of type R³⁰CO₂H under a standard carboxylic acid and amine coupling procedure known to one skilled in the art. In an example, compound 2.10, R³⁰CO₂H, HATU (or other appropriate amine-carboxylic acid coupling agent, e.g., DCC or PS-DCC in the presence of HOBt) are combined, and then DIEA is added. The mixture is diluted with an appropriate solvent(s) (e.g., 2:1 CH₂Cl₂: DMF) to an appropriate solution concentration, and allowed to stir at ambient temperature (about 15-30° C.) for a period of time sufficient to complete the reaction, e.g., about 4 h. The product (2.11) is isolated by methods known to one skilled in the art (e.g., filtering by vacuum to collect the precipitated product; followed by purification, e.g., chromatography, if necessary).

3. Route III

In one aspect, substituted 2-oxo-2,3-dihydro-1H-benzo[d]imidazol-1-yl analogs of the present invention can be prepared generically by the synthetic scheme as shown below.

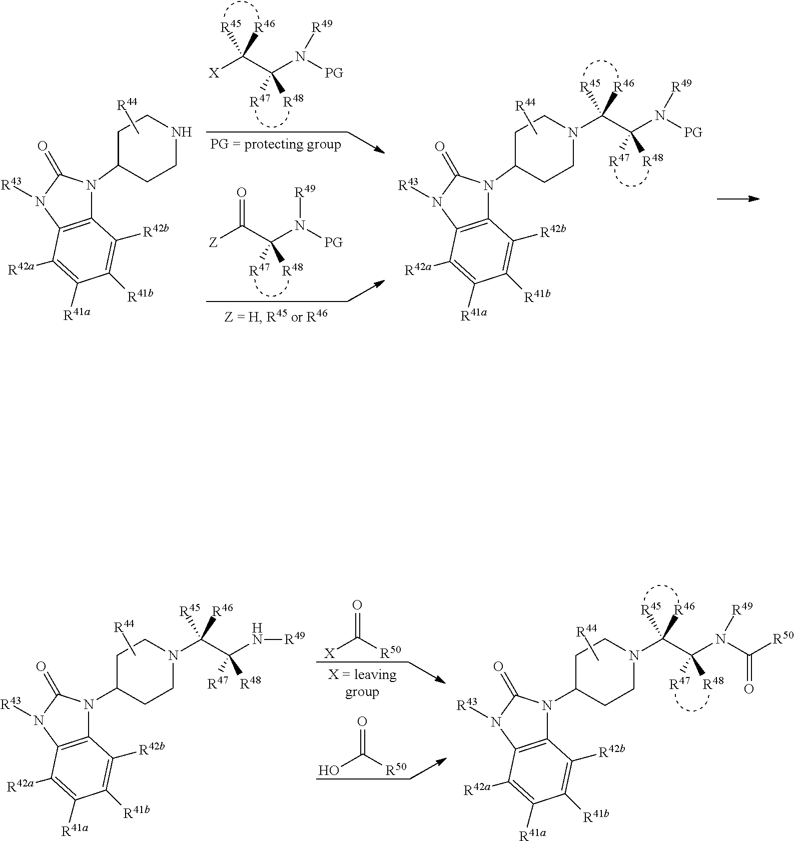

Compounds are represented in generic form, with substituents as noted in compound descriptions elsewhere herein. A more specific example is set forth below.

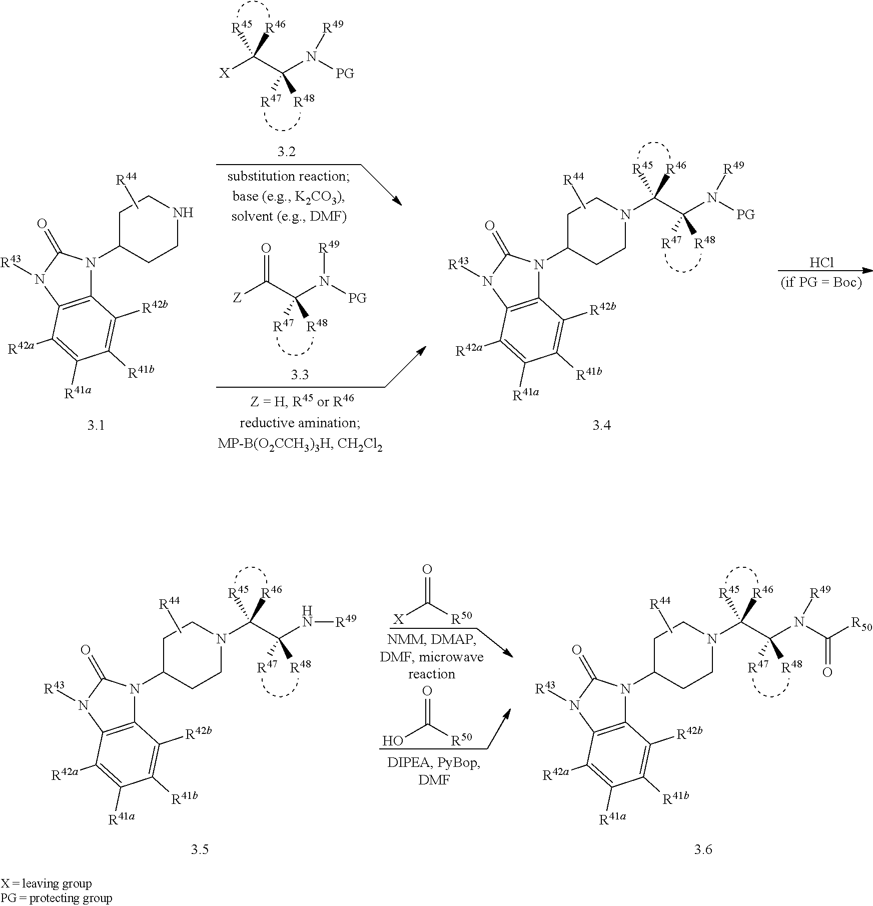

In one aspect, Route III begins with a suitable substituted compound of type 3.1. A suitable 1-(piperidin-4-yl)-1H-benzo[d]imidazol-2(3H)-one derivative (3.1) is commercially available or can be readily prepared by one skilled in the art. In one aspect, the reaction of 3.1 and 3.2 is typically carried out under a suitable reaction atmosphere and in a suitable solvent that supports substitution reactions such as DMF in the presence of an appropriate base such as K₂CO₃. The reaction is conducted at a suitable temperature and for a time sufficient to complete the reaction, to provide compounds of type 3.4 as shown above. The product, a compound of type 3.4, is isolated by methods known to one skilled in the art (e.g., extraction, washing, drying, and concentration under a vacuum; followed by purification, e.g., chromatography, if necessary).

In one aspect, the reaction of 3.1 and 3.3 is typically carried out under a suitable reaction condition that supports reductive amination of carbonyl compounds known to one skilled in the art to give products of type 3.4. Reaction components 3.1 and 3.3 are dissolved in a suitable solvent, e.g., dichloromethane and stirred Then, the reducing agent, [e.g., macroporous polystyrene triacetoxyborohydride, MP-B(O₂CCH₃)₃H. or other suitable reducing agent] is added to the reaction mixture. The reaction is carried out for a time sufficient to complete the reaction, e.g., 16 h, to provide compounds of type 3.4 as shown above. The product, a compound of type 3.4, is isolated by methods known to one skilled in the art (e.g., filtered, extracted, and concentration under a vacuum; followed by purification, e.g., chromatography, if necessary).

In one aspect, compounds of type 3.5 can be prepared by the conversion of the N-protected compound (e.g., N-Boc compound type 3.4) to the corresponding amine derivative (3.5). For example, a reaction of this type is commonly carried out by dissolving the N-Boc derivative (2.9) in a suitable solvent(s) (e.g., 1,2-dichloroethane/methanol) and then HCl (e.g., 4 M HCl in dioxane) is added. The mixture is stirred for a time sufficient, e.g., about 16 h, at ambient room temperature (about 15 to 30° C.) to complete the reaction. The product (3.5) is isolated by methods known to one skilled in the art (e.g., concentration under a vacuum; followed by purification, e.g., chromatography).

In one aspect, compounds of type 3.6 can be prepared by the acylation of 3.5 with an appropriate acid halide of type R⁵⁰C(O)X under a standard amine acylation procedure known to one skilled in the art. In an example, compound 3.5 is dissolved in a suitable solvent such as DMF; N-methylmorpholine is added, R⁵⁰C(O)X is added; and a catalytic amount of DMAP is added. The mixture is reacted under microwave irradiation for about 17 min or sufficient time and at an appropriate temperature (about 155° C.) to complete the reaction. The product (3.6) is isolated by methods known to one skilled in the art (e.g., concentration under a vacuum; followed by purification, e.g., chromatography).

In one aspect, compounds of type 3.6 can be prepared by the acylation of 3.5 with an appropriate carboxylic acid of type R⁵⁰CO₂H under a standard amine acylation procedure known to one skilled in the art. In an example, compound 3.5 is dissolved in a suitable solvent such as DMF; R⁵⁰CO₂H is added; an appropriate base, e.g., N,N-diisopropylamine (DIEA), is added; and (benzotriazol-1-lyoxy)tripyrrolidinophosphonium hexafluorophosphate (PyBOP) is added. The mixture is allowed to stir/rotate for about 16 h. or sufficient time and at ambient temperature (about 15-30° C.) to complete the reaction. The product (3.6) is isolated by methods known to one skilled in the art (e.g., concentration under a vacuum; followed by purification, e.g., chromatography).

It is understood that the disclosed methods of making can be used in connection with the disclosed compounds, compositions, kits, and uses.

### E. PHARMACEUTICAL COMPOSITIONS

In one aspect, the invention relates to pharmaceutical compositions comprising the disclosed compounds. That is, a pharmaceutical composition can be provided comprising a therapeutically effective amount of at least one disclosed compound or at least one product of a disclosed method and a pharmaceutically acceptable carrier.

In one aspect, the invention relates to a pharmaceutical composition comprising an effective amount of a phospholipase D inhibitor, or a pharmaceutically acceptable prodrug, salt, solvate, or polymorph thereof; an effective amount of at least one compound selected from: a) an HIV fusion/lysis inhibitor, or a pharmaceutically acceptable prodrug, salt, solvate, or polymorph thereof; b) an HIV integrase inhibitor, or a pharmaceutically acceptable prodrug, salt, solvate, or polymorph thereof; c) an HIV non-nucleoside reverse transcriptase inhibitor, or a pharmaceutically acceptable prodrug, salt, solvate, or polymorph thereof; d) an HIV nucleoside reverse transcriptase inhibitor, or a pharmaceutically acceptable prodrug, salt, solvate, or polymorph thereof; and e) an HIV protease inhibitor, or a pharmaceutically acceptable prodrug, salt, solvate; and a pharmaceutically acceptable carrier.

In a further aspect, the effective amount is a therapeutically effective amount. In a still further aspect, the effective amount is a prophylactically effective amount.

In a further aspect, the effective amount of the phospholipase D inhibitor inhibits HIV replication. In a still further aspect, the effective amount of the phospholipase D inhibitor inhibits HIV integration.

In a further aspect, the phospholipase D inhibitor is a disclosed phospholipase D inhibitor. In a still further aspect, the phospholipase D inhibitor inhibits PLD1 and/or PLD2. In yet a further aspect, the phospholipase D inhibitor inhibits PLD1. In an even further aspect, the phospholipase D inhibitor inhibits PLD2.

In a further aspect, the phospholipase D inhibitor is selected from:

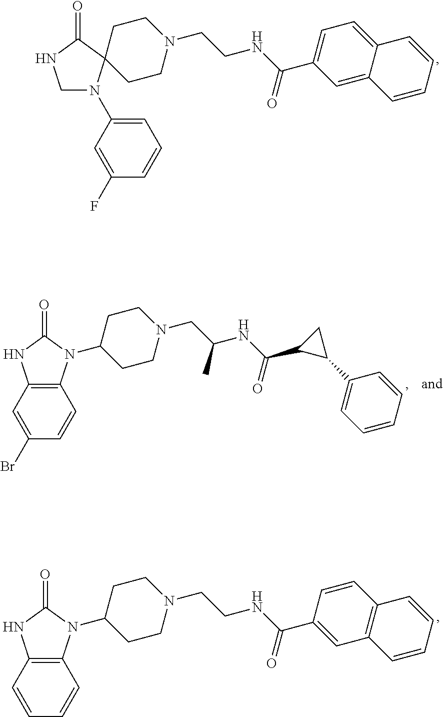

or a pharmaceutically acceptable salt, hydrate, solvate, or polymorph thereof.

In a further aspect, the HIV fusion/lysis inhibitor of the composition is selected from enfuvirtide, maraviroc, cenicriviroc, ibalizumab, BMS-663068, and PRO-140, or a pharmaceutically acceptable prodrug, salt, solvate, or polymorph thereof. In a still further aspect, the HIV fusion/lysis inhibitor is selected from enfuvirtide, maraviroc, cenicriviroc, and ibalizumab, or a pharmaceutically acceptable prodrug, salt, solvate, or polymorph thereof. In yet a further aspect, the HIV fusion/lysis inhibitor is enfuvirtide, or a pharmaceutically acceptable prodrug, salt, solvate, or polymorph thereof. In an even further aspect, the HIV fusion/lysis inhibitor is maraviroc, or a pharmaceutically acceptable prodrug, salt, solvate, or polymorph thereof. In a still further aspect, the HIV fusion/lysis inhibitor is cenicriviroc, or a pharmaceutically acceptable prodrug, salt, solvate, or polymorph thereof. In yet a further aspect, the HIV fusion/lysis inhibitor is ibalizumab, or a pharmaceutically acceptable prodrug, salt, solvate, or polymorph thereof.

In a further aspect, the HIV integrase inhibitor of the composition is selected from raltegravir, dolutegravir, elvitegravir, and S/GSK1265744, or a pharmaceutically acceptable prodrug, salt, solvate, or polymorph thereof. In a still further aspect, the HIV integrase inhibitor is selected from raltegravir, dolutegravir, and elvitegravir, or a pharmaceutically acceptable prodrug, salt, solvate, or polymorph thereof. In yet a further aspect, the HIV integrase inhibitor is raltegravir, or a pharmaceutically acceptable prodrug, salt, solvate, or polymorph thereof. In an even further aspect, the HIV integrase inhibitor is dolutegravir, or a pharmaceutically acceptable prodrug, salt, solvate, or polymorph thereof. In a still further aspect, the HIV integrase inhibitor is elvitegravir, or a pharmaceutically acceptable prodrug, salt, solvate, or polymorph thereof.

In a further aspect, the non-nucleoside reverse transcriptase inhibitor of the composition is selected from delavirdine, efavirenz, etravirine, nevirapine, rilpivirine, and lersivirine, or a pharmaceutically acceptable prodrug, salt, solvate, or polymorph thereof. In a still further aspect, the non-nucleoside reverse transcriptase inhibitor is delavirdine, or a pharmaceutically acceptable prodrug, salt, solvate, or polymorph thereof. In yet a further aspect, the non-nucleoside reverse transcriptase inhibitor is efavirenz, or a pharmaceutically acceptable prodrug, salt, solvate, or polymorph thereof. In an even further aspect, the non-nucleoside reverse transcriptase inhibitor is etravirine, or a pharmaceutically acceptable prodrug, salt, solvate, or polymorph thereof. In a still further aspect, the non-nucleoside reverse transcriptase inhibitor is nevirapine, or a pharmaceutically acceptable prodrug, salt, solvate, or polymorph thereof. In yet a further aspect, the non-nucleoside reverse transcriptase inhibitor is rilpivirine, or a pharmaceutically acceptable prodrug, salt, solvate, or polymorph thereof.

In a further aspect, the nucleoside reverse transcriptase inhibitor of the composition is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In a still further aspect, the nucleoside reverse transcriptase inhibitor is selected from abacavir, didansine, emtricitabine, lamivudine, stavudine, tenofovir, zidovudine, and elvucitabine, or a pharmaceutically acceptable prodrug, salt, solvate, or polymorph thereof. In yet a further aspect, the nucleoside reverse transcriptase inhibitor is abacavir, or a pharmaceutically acceptable prodrug, salt, solvate, or polymorph thereof. In an even further aspect, the nucleoside reverse transcriptase inhibitor is didansine, or a pharmaceutically acceptable prodrug, salt, solvate, or polymorph thereof. In a still further aspect, the nucleoside reverse transcriptase inhibitor is elvucitabine, or a pharmaceutically acceptable prodrug, salt, solvate, or polymorph thereof. In yet a further aspect, the nucleoside reverse transcriptase inhibitor is emtricitabine, or a pharmaceutically acceptable prodrug, salt, solvate, or polymorph thereof. In an even further aspect, the nucleoside reverse transcriptase inhibitor is lamivudine, or a pharmaceutically acceptable prodrug, salt, solvate, or polymorph thereof. In a still further aspect, the nucleoside reverse transcriptase inhibitor is stavudine, or a pharmaceutically acceptable prodrug, salt, solvate, or polymorph thereof. In yet a further aspect, the nucleoside reverse transcriptase inhibitor is tenofovir, or a pharmaceutically acceptable prodrug, salt, solvate, or polymorph thereof. In an even further aspect, the nucleoside reverse transcriptase inhibitor is zidovudine, or a pharmaceutically acceptable prodrug, salt, solvate, or polymorph thereof.

In a further aspect, the protease inhibitor of the composition is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In a still further aspect, the protease inhibitor is atazanir, or a pharmaceutically acceptable prodrug, salt, solvate, or polymorph thereof. In yet a further aspect, the protease inhibitor is darunavir, or a pharmaceutically acceptable prodrug, salt, solvate, or polymorph thereof. In an even further aspect, the protease inhibitor is fosamprenavir, or a pharmaceutically acceptable prodrug, salt, solvate, or polymorph thereof. In a still further aspect, the protease inhibitor is indinavir, or a pharmaceutically acceptable prodrug, salt, solvate, or polymorph thereof. In yet a further aspect, the protease inhibitor is lopinavir, or a pharmaceutically acceptable prodrug, salt, solvate, or polymorph thereof. In an even further aspect, the protease inhibitor is nelfinavir, or a pharmaceutically acceptable prodrug, salt, solvate, or polymorph thereof. In a still further aspect, the protease inhibitor is ritonavir, or a pharmaceutically acceptable prodrug, salt, solvate, or polymorph thereof. In yet a further aspect, the protease inhibitor is saquinavir, or a pharmaceutically acceptable prodrug, salt, solvate, or polymorph thereof. In an even further aspect, the protease inhibitor is tipranavir, or a pharmaceutically acceptable prodrug, salt, solvate, or polymorph thereof.

In one aspect, the invention relates to a pharmaceutical composition comprising: a) a first antiviral agent comprising an effective amount of a phospholipase D inhibitor, or a pharmaceutically acceptable prodrug, salt, solvate, or polymorph thereof; b) a second antiviral agent comprising an HIV non-nucleoside reverse transcriptase inhibitor, or a pharmaceutically acceptable prodrug, salt, solvate, or polymorph thereof; and c) a third antiviral agent comprising an HIV nucleoside reverse transcriptase inhibitor, or a pharmaceutically acceptable prodrug, salt, solvate, or polymorph thereof.

In a further aspect, the first antiviral agent of the composition is a disclosed phospholipase D inhibitor, or a pharmaceutically acceptable prodrug, salt, solvate, or polymorph thereof.

In a further aspect, the second antiviral agent of the composition is selected from delavirdine, efavirenz, etravirine, nevirapine, rilpivirine, and lersivirine, or a pharmaceutically acceptable salt, solvate, or polymorph thereof.

In a further aspect, the third antiviral agent of the composition is selected from abacavir, didansine, emtricitabine, lamivudine, stavudine, tenofovir, zidovudine, elvucitabine, and GS-7340, or a pharmaceutically acceptable salt, solvate, or polymorph thereof.

In a further aspect, the composition further comprises a fourth antiviral agent, wherein the fifth antiviral agent is a nucleoside reverse transcriptase inhibitor, or a pharmaceutically acceptable prodrug, salt, solvate, or polymorph thereof, that is distinct from the third antiviral agent.

In a further aspect, the first antiviral agent is a disclosed phospholipase D inhibitor; wherein the second antiviral agent is selected from delavirdine, efavirenz, etravirine, nevirapine, rilpivirine, and lersivirine, or a pharmaceutically acceptable prodrug, salt, solvate, or polymorph thereof; and wherein third antiviral agent is selected from abacavir, didansine, emtricitabine, lamivudine, stavudine, tenofovir, zidovudine, elvucitabine, or a pharmaceutically acceptable prodrug, salt, solvate, or polymorph thereof.

In a further aspect, the composition is formulated for oral administration. In a still further aspect, the composition is formulated for intravenous administration.

In one aspect, the invention relates to a pharmaceutical composition comprising: a) a first antiviral agent comprising an effective amount of a phospholipase D inhibitor, or a pharmaceutically acceptable prodrug, salt, solvate, or polymorph thereof; b) a second antiviral agent comprising an HIV fusion/lysis inhibitor, or a pharmaceutically acceptable prodrug, salt, solvate, or polymorph thereof; and c) a third antiviral agent comprising an HIV integrase inhibitor, or a pharmaceutically acceptable prodrug, salt, solvate, or polymorph thereof; and a pharmaceutically acceptable carrier.

In a further aspect, the first antiviral agent is a disclosed phospholipase D inhibitor, or a pharmaceutically acceptable prodrug, salt, solvate, or polymorph thereof.

In a further aspect, the second antiviral agent is selected from enfuvirtide, maraviroc, cenicriviroc, ibalizumab, BMS-663068, and PRO-140, or a pharmaceutically acceptable salt, solvate, or polymorph thereof.

In a further aspect, the third antiviral agent is selected from raltegravir, dolutegravir, elvitegravir, and S/GSK1265744, or a pharmaceutically acceptable salt, solvate, or polymorph thereof.

In a further aspect, the first antiviral agent is a disclosed phospholipase D inhibitor; wherein the second antiviral agent is selected from enfuvirtide, maraviroc, cenicriviroc, and ibalizumab, or a pharmaceutically acceptable prodrug, salt, solvate, or polymorph thereof; and wherein third antiviral agent is selected from raltegravir, dolutegravir, and elvitegravir, or a pharmaceutically acceptable prodrug, salt, solvate, or polymorph thereof.

In a further aspect, the composition is formulated for oral administration. In a still further aspect, the composition is formulated for intravenous administration.

In one aspect, the invention relates to a pharmaceutical composition comprising: a) a first antiviral agent comprising an effective amount of a phospholipase D inhibitor, or a pharmaceutically acceptable prodrug, salt, solvate, or polymorph thereof; b) a second antiviral agent comprising an HIV fusion/lysis inhibitor, or a pharmaceutically acceptable prodrug, salt, solvate, or polymorph thereof; and c) a third antiviral agent comprising an HIV protease inhibitor, or a pharmaceutically acceptable prodrug, salt, solvate, or polymorph thereof; and a pharmaceutically acceptable carrier.

In a further aspect, the first antiviral agent is a disclosed phospholipase D inhibitor, or a pharmaceutically acceptable prodrug, salt, solvate, or polymorph thereof.

In a further aspect, the second antiviral agent is selected from enfuvirtide, maraviroc, cenicriviroc, ibalizumab, BMS-663068, and PRO-140, or a pharmaceutically acceptable salt, solvate, or polymorph thereof.

In a further aspect, the third antiviral agent is selected from atazanavir, darunavir, fosamprenavir, indinavir, lopinavir/ritonavir, nelfinavir, ritonavir, saquinavir, and tipranavir, or a pharmaceutically acceptable salt, solvate, or polymorph thereof.

In a further aspect, the first antiviral agent is a disclosed phospholipase D inhibitor; wherein the second antiviral agent is selected from enfuvirtide, maraviroc, cenicriviroc, and ibalizumab, or a pharmaceutically acceptable prodrug, salt, solvate, or polymorph thereof; and wherein third antiviral agent is selected from atazanavir, darunavir, fosamprenavir, indinavir, lopinavir/ritonavir, nelfinavir, ritonavir, saquinavir, and tipranavir, or a pharmaceutically acceptable prodrug, salt, solvate, or polymorph thereof.

In a further aspect, the composition is formulated for oral administration. In a still further aspect, the composition is formulated for intravenous administration.

In one aspect, the invention relates to a pharmaceutical composition comprising: a) a first antiviral agent comprising an effective amount of a phospholipase D inhibitor, or a pharmaceutically acceptable prodrug, salt, solvate, or polymorph thereof; and b) a second antiviral agent comprising an HIV fusion/lysis inhibitor, or a pharmaceutically acceptable prodrug, salt, solvate, or polymorph thereof.

In a further aspect, the first antiviral agent is a disclosed phospholipase D inhibitor, or a pharmaceutically acceptable prodrug, salt, solvate, or polymorph thereof. In a still further aspect, the first antiviral agent is a disclosed phospholipase D inhibitor; wherein the second antiviral agent is selected from enfuvirtide, maraviroc, cenicriviroc, and ibalizumab, or a pharmaceutically acceptable prodrug, salt, solvate, or polymorph thereof. In yet a further aspect, the first antiviral agent is a disclosed phospholipase D inhibitor; and wherein the second antiviral agent is enfuvirtide, or a pharmaceutically acceptable prodrug, salt, solvate, or polymorph thereof. In an even further aspect, the first antiviral agent is a disclosed phospholipase D inhibitor; and wherein the second antiviral agent is maraviroc, or a pharmaceutically acceptable prodrug, salt, solvate, or polymorph thereof. In a still further aspect, the first antiviral agent is a disclosed phospholipase D inhibitor; and wherein the second antiviral agent is cenicriviroc, or a pharmaceutically acceptable prodrug, salt, solvate, or polymorph thereof. In yet a further aspect, the first antiviral agent is a disclosed phospholipase D inhibitor; and wherein the second antiviral agent is ibalizumab, or a pharmaceutically acceptable prodrug, salt, solvate, or polymorph thereof.

In a further aspect, the composition is formulated for oral administration. In a still further aspect, the composition is formulated for intravenous administration.

In one aspect, the invention relates to a pharmaceutical composition comprising: a) a first antiviral agent comprising an effective amount of a phospholipase D inhibitor, or a pharmaceutically acceptable prodrug, salt, solvate, or polymorph thereof; and b) a second antiviral agent comprising an HIV integrase inhibitor, or a pharmaceutically acceptable prodrug, salt, solvate, or polymorph thereof.

In a further aspect, the first antiviral agent is a disclosed phospholipase D inhibitor, or a pharmaceutically acceptable prodrug, salt, solvate, or polymorph thereof. In a still further aspect, the first antiviral agent is a disclosed phospholipase D inhibitor; wherein the second antiviral agent is selected from raltegravir, dolutegravir, and elvitegravir, or a pharmaceutically acceptable prodrug, salt, solvate, or polymorph thereof. In yet a further aspect, the first antiviral agent is a disclosed phospholipase D inhibitor; and wherein the second antiviral agent is raltegravir, or a pharmaceutically acceptable prodrug, salt, solvate, or polymorph thereof. In an even further aspect, the first antiviral agent is a disclosed phospholipase D inhibitor; and wherein the second antiviral agent is dolutegravir, or a pharmaceutically acceptable prodrug, salt, solvate, or polymorph thereof. In a still further aspect, the first antiviral agent is a disclosed phospholipase D inhibitor; and wherein the second antiviral agent is elvitegravir, or a pharmaceutically acceptable prodrug, salt, solvate, or polymorph thereof.

In a further aspect, the composition is formulated for oral administration. In a still further aspect, the composition is formulated for intravenous administration.

In one aspect, the invention relates to a pharmaceutical composition comprising: a) a first antiviral agent comprising an effective amount of a phospholipase D inhibitor, or a pharmaceutically acceptable prodrug, salt, solvate, or polymorph thereof; and b) a second antiviral agent comprising an HIV non-nucleoside reverse transcriptase inhibitor, or a pharmaceutically acceptable prodrug, salt, solvate, or polymorph thereof.

In a further aspect, the first antiviral agent is a disclosed phospholipase D inhibitor, or a pharmaceutically acceptable prodrug, salt, solvate, or polymorph thereof. In a still further aspect, the first antiviral agent is a disclosed phospholipase D inhibitor; wherein the second antiviral agent is selected from delavirdine, efavirenz, etravirine, nevirapine, rilpivirine, and lersivirine, or a pharmaceutically acceptable prodrug, salt, solvate, or polymorph thereof. In yet a further aspect, the first antiviral agent is a disclosed phospholipase D inhibitor; and wherein the second antiviral agent is delavirdine, or a pharmaceutically acceptable prodrug, salt, solvate, or polymorph thereof. In an even further aspect, the first antiviral agent is a disclosed phospholipase D inhibitor; and wherein the second antiviral agent is efavirenz, or a pharmaceutically acceptable prodrug, salt, solvate, or polymorph thereof. In a still further aspect, the first antiviral agent is a disclosed phospholipase D inhibitor; and wherein the second antiviral agent is etravirine, or a pharmaceutically acceptable prodrug, salt, solvate, or polymorph thereof. In yet a further aspect, the first antiviral agent is a disclosed phospholipase D inhibitor; and wherein the second antiviral agent is nevirapine, or a pharmaceutically acceptable prodrug, salt, solvate, or polymorph thereof. In an even further aspect, the first antiviral agent is a disclosed phospholipase D inhibitor; and wherein the second antiviral agent is rilpivirine, or a pharmaceutically acceptable prodrug, salt, solvate, or polymorph thereof. In a still further aspect, the first antiviral agent is a disclosed phospholipase D inhibitor; and wherein the second antiviral agent is lersivirine, or a pharmaceutically acceptable prodrug, salt, solvate, or polymorph thereof.

In a further aspect, the composition is formulated for oral administration. In a still further aspect, the composition is formulated for intravenous administration.

In one aspect, the invention relates to a pharmaceutical composition comprising: a) a first antiviral agent comprising an effective amount of a phospholipase D inhibitor, or a pharmaceutically acceptable prodrug, salt, solvate, or polymorph thereof; and b) a second antiviral agent comprising an HIV nucleoside reverse transcriptase inhibitor, or a pharmaceutically acceptable prodrug, salt, solvate, or polymorph thereof.

In a further aspect, the first antiviral agent is a disclosed phospholipase D inhibitor, or a pharmaceutically acceptable prodrug, salt, solvate, or polymorph thereof. In a still further aspect, the first antiviral agent is a disclosed phospholipase D inhibitor; wherein the second antiviral agent is selected from abacavir, didansine, emtricitabine, lamivudine, stavudine, tenofovir, zidovudine, and elvucitabine, or a pharmaceutically acceptable prodrug, salt, solvate, or polymorph thereof. In yet a further aspect, the first antiviral agent is a disclosed phospholipase D inhibitor; and wherein the second antiviral agent is abacavir, or a pharmaceutically acceptable prodrug, salt, solvate, or polymorph thereof. In an even further aspect, the first antiviral agent is a disclosed phospholipase D inhibitor; and wherein the second antiviral agent is didansine, or a pharmaceutically acceptable prodrug, salt, solvate, or polymorph thereof. In a still further aspect, the first antiviral agent is a disclosed phospholipase D inhibitor; and wherein the second antiviral agent is emtricitabine, or a pharmaceutically acceptable prodrug, salt, solvate, or polymorph thereof. In yet a further aspect, the first antiviral agent is a disclosed phospholipase D inhibitor; and wherein the second antiviral agent is lamivudine, or a pharmaceutically acceptable prodrug, salt, solvate, or polymorph thereof. In an even further aspect, the first antiviral agent is a disclosed phospholipase D inhibitor; and wherein the second antiviral agent is stavudine, or a pharmaceutically acceptable prodrug, salt, solvate, or polymorph thereof. In an even further aspect, the first antiviral agent is a disclosed phospholipase D inhibitor; and wherein the second antiviral agent is tenofovir, or a pharmaceutically acceptable prodrug, salt, solvate, or polymorph thereof. In a still further aspect, the first antiviral agent is a disclosed phospholipase D inhibitor; and wherein the second antiviral agent is zidovudine, or a pharmaceutically acceptable prodrug, salt, solvate, or polymorph thereof. In yet a further aspect, the first antiviral agent is a disclosed phospholipase D inhibitor; and wherein the second antiviral agent is elvucitabine, or a pharmaceutically acceptable prodrug, salt, solvate, or polymorph thereof.

In a further aspect, the composition is formulated for oral administration. In a still further aspect, the composition is formulated for intravenous administration.

In one aspect, the invention relates to a pharmaceutical composition comprising: a) a first antiviral agent comprising an effective amount of a phospholipase D inhibitor, or a pharmaceutically acceptable prodrug, salt, solvate, or polymorph thereof; and b) a second antiviral agent comprising an HIV protease inhibitor, or a pharmaceutically acceptable prodrug, salt, solvate, or polymorph thereof.

In a further aspect, the first antiviral agent is a disclosed phospholipase D inhibitor, or a pharmaceutically acceptable prodrug, salt, solvate, or polymorph thereof. In a still further aspect, the first antiviral agent is a disclosed phospholipase D inhibitor; wherein the second antiviral agent is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In yet a further aspect, the first antiviral agent is a disclosed phospholipase D inhibitor; and wherein the second antiviral agent is atazanavir, or a pharmaceutically acceptable prodrug, salt, solvate, or polymorph thereof. In an even further aspect, the first antiviral agent is a disclosed phospholipase D inhibitor; and wherein the second antiviral agent is darunavir, or a pharmaceutically acceptable prodrug, salt, solvate, or polymorph thereof. In a still further aspect, the first antiviral agent is a disclosed phospholipase D inhibitor; and wherein the second antiviral agent is fosamprenavir, or a pharmaceutically acceptable prodrug, salt, solvate, or polymorph thereof. In yet a further aspect, the first antiviral agent is a disclosed phospholipase D inhibitor; and wherein the second antiviral agent is indinavir, or a pharmaceutically acceptable prodrug, salt, solvate, or polymorph thereof. In an even further aspect, the first antiviral agent is a disclosed phospholipase D inhibitor; and wherein the second antiviral agent is lopinavir, or a pharmaceutically acceptable prodrug, salt, solvate, or polymorph thereof. In a still further aspect, the first antiviral agent is a disclosed phospholipase D inhibitor; and wherein the second antiviral agent is nelfinavir, or a pharmaceutically acceptable prodrug, salt, solvate, or polymorph thereof. In yet a further aspect, the first antiviral agent is a disclosed phospholipase D inhibitor; and wherein the second antiviral agent is ritonavir, or a pharmaceutically acceptable prodrug, salt, solvate, or polymorph thereof. In an even further aspect, the first antiviral agent is a disclosed phospholipase D inhibitor; and wherein the second antiviral agent is saquinavir, or a pharmaceutically acceptable prodrug, salt, solvate, or polymorph thereof. In a still further aspect, the first antiviral agent is a disclosed phospholipase D inhibitor; and wherein the second antiviral agent is tipranavir, or a pharmaceutically acceptable prodrug, salt, solvate, or polymorph thereof. In yet a further aspect, the first antiviral agent is a disclosed phospholipase D inhibitor; and wherein the second antiviral agent is lopinavir/ritonavir, or a pharmaceutically acceptable prodrug, salt, solvate, or polymorph thereof.

In a further aspect, the composition is formulated for oral administration. In a still further aspect, the composition is formulated for intravenous administration.

The disclosed compounds can be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and can be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans. The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions encompass any composition made by admixing a compound of the present invention and a pharmaceutically acceptable carrier.

As used herein, the term “pharmaceutically acceptable salts” refers to salts prepared from pharmaceutically acceptable non-toxic bases or acids. When a disclosed compound is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids” includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable derivatives thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

Pharmaceutical compositions suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In the treatment of the disclosed conditions, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level can be about 0.01 to 250 mg/kg per day, about 0.05 to 100 mg/kg per day, or about 0.1 to 50 mg/kg per day. Within this range the dosage can be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The disclosed pharmaceutical compositions can further comprise other therapeutically active compounds, as discussed further herein, which are usually applied in the treatment of the above mentioned pathological conditions.

In a further aspect, a pharmaceutical composition can comprise a therapeutically effective amount of any one or more disclosed compound and a pharmaceutically acceptable carrier. In a further aspect, a pharmaceutical composition can comprise a therapeutically effective amount of one or more product of any disclosed method and a pharmaceutically acceptable carrier. In one aspect, the invention relates to a method for manufacturing a medicament comprising combining at least one disclosed compound or at least one product of a disclosed method with a pharmaceutically acceptable carrier or diluent.

It is understood that the disclosed compositions can be prepared from the disclosed compounds. It is also understood that the disclosed compositions can be employed in the disclosed methods of using.

### F. KITS

In one aspect, the invention relates to a kit comprising at least one disclosed compound or at least one product of a disclosed method and at least one agent known to increase PLD activity. In a further aspect, a kit comprises at least one disclosed compound or at least one product of a disclosed method and at least one agent known to decrease PLD activity. In a further aspect, the at least one compound or the at least one product and the at least one agent are co-formulated. In a further aspect, the at least one compound or the at least one product and the at least one agent are co-packaged.

In one aspect, the invention relates to a kit comprising a phospholipase D inhibitor, or a pharmaceutically acceptable prodrug, salt, solvate, or polymorph thereof, and one or more of: a) at least one agent known to treat an HIV infection; b) at least one agent known to treat an opportunistic infection associated with an HIV infection; c) instructions for treating an HIV infection; d) instructions for treating an opportunistic infection associated with an HIV infection; e) instructions for administering the phospholipase D inhibitor in connection with treating an HIV infection; or f) instructions for administering the phospholipase D inhibitor in connection with reducing the risk of HIV infection.

In a further aspect, the phospholipase D inhibitor and the at least one agent are co-packaged. In a still further aspect, the phospholipase D inhibitor and the at least one agent are co-formulated.

In a further aspect, the kit further comprises a plurality of dosage forms, the plurality comprising one or more doses; wherein each dose comprises an effective amount of the phospholipase D inhibitor and the at least one agent.

In a further aspect, the effective amount is a therapeutically effective amount. In a still further aspect, the effective amount is a prophylactically effective amount.

In a further aspect, each dose of the phospholipase D inhibitor and the at least one agent are co-formulated. In a still further aspect, each dose of the phospholipase D inhibitor and the at least one agent are co-packaged.

In a further aspect, the dosage forms are formulated for oral administration and/or intravenous administration. In a still further aspect, the dosage forms are formulated for oral administration. In yet a further aspect, the dosage forms are formulated for intravenous administration.

In a further aspect, the dosage form for the phospholipase D inhibitor is formulated for oral administration and the dosage for the at least one agent is formulated for intravenous administration. In a still further aspect, the dosage form for the phospholipase D inhibitor is formulated for intravenous administration and the dosage for the at least one agent is formulated for oral administration.

In a further aspect, the phospholipase D inhibitor is a disclosed phospholipase D inhibitor. In a still further aspect, the phospholipase D inhibitor inhibits PLD1 and/or PLD2. In yet a further aspect, the phospholipase D inhibitor inhibits PLD1. In an even further aspect, the phospholipase D inhibitor inhibits PLD2.

In a further aspect, the at least one agent is selected from an HIV fusion/lysis inhibitor, or a pharmaceutically acceptable prodrug, salt, solvate, or polymorph thereof; an HIV integrase inhibitor, or a pharmaceutically acceptable prodrug, salt, solvate, or polymorph thereof an HIV non-nucleoside reverse transcriptase inhibitor, or a pharmaceutically acceptable prodrug, salt, solvate, or polymorph thereof; an HIV nucleoside reverse transcriptase inhibitor, or a pharmaceutically acceptable prodrug, salt, solvate, or polymorph thereof; and an HIV protease inhibitor, or a pharmaceutically acceptable prodrug, salt, solvate, or polymorph thereof.

In a further aspect, the HIV fusion/lysis inhibitor is selected from enfuvirtide, maraviroc, cenicriviroc, ibalizumab, BMS-663068, and PRO-140, or a pharmaceutically acceptable prodrug, salt, solvate, or polymorph thereof. In a still further aspect, the HIV fusion/lysis inhibitor is selected from enfuvirtide, maraviroc, cenicriviroc, and ibalizumab, or a pharmaceutically acceptable prodrug, salt, solvate, or polymorph thereof. In yet a further aspect, the HIV fusion/lysis inhibitor is enfuvirtide, or a pharmaceutically acceptable prodrug, salt, solvate, or polymorph thereof. In an even further aspect, the HIV fusion/lysis inhibitor is maraviroc, or a pharmaceutically acceptable prodrug, salt, solvate, or polymorph thereof. In a still further aspect, the HIV fusion/lysis inhibitor is cenicriviroc, or a pharmaceutically acceptable prodrug, salt, solvate, or polymorph thereof. In yet a further aspect, the HIV fusion/lysis inhibitor is ibalizumab, or a pharmaceutically acceptable prodrug, salt, solvate, or polymorph thereof.

In a further aspect, the HIV integrase inhibitor is selected from raltegravir, dolutegravir, elvitegravir, and S/GSK1265744, or a pharmaceutically acceptable prodrug, salt, solvate, or polymorph thereof. In a still further aspect, the HIV integrase inhibitor is selected from raltegravir, dolutegravir, and elvitegravir, or a pharmaceutically acceptable prodrug, salt, solvate, or polymorph thereof. In yet a further aspect, the HIV integrase inhibitor is raltegravir, or a pharmaceutically acceptable prodrug, salt, solvate, or polymorph thereof. In an even further aspect, the HIV integrase inhibitor is dolutegravir, or a pharmaceutically acceptable prodrug, salt, solvate, or polymorph thereof. In a still further aspect, the HIV integrase inhibitor is elvitegravir, or a pharmaceutically acceptable prodrug, salt, solvate, or polymorph thereof.

In a further aspect, the non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In a still further aspect, the non-nucleoside reverse transcriptase inhibitor is delavirdine, or a pharmaceutically acceptable prodrug, salt, solvate, or polymorph thereof. In yet a further aspect, the non-nucleoside reverse transcriptase inhibitor is efavirenz, or a pharmaceutically acceptable prodrug, salt, solvate, or polymorph thereof. In an even further aspect, the non-nucleoside reverse transcriptase inhibitor is etravirine, or a pharmaceutically acceptable prodrug, salt, solvate, or polymorph thereof. In a still further aspect, the non-nucleoside reverse transcriptase inhibitor is nevirapine, or a pharmaceutically acceptable prodrug, salt, solvate, or polymorph thereof. In yet a further aspect, the non-nucleoside reverse transcriptase inhibitor is rilpivirine, or a pharmaceutically acceptable prodrug, salt, solvate, or polymorph thereof. In an even further aspect, the non-nucleoside reverse transcriptase inhibitor is lersivirine, or a pharmaceutically acceptable prodrug, salt, solvate, or polymorph thereof.

In a further aspect, the nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In a still further aspect, the nucleoside reverse transcriptase inhibitor is selected from abacavir, didansine, emtricitabine, lamivudine, stavudine, tenofovir, zidovudine, and elvucitabine, or a pharmaceutically acceptable prodrug, salt, solvate, or polymorph thereof. In yet a further aspect, the nucleoside reverse transcriptase inhibitor is abacavir, or a pharmaceutically acceptable prodrug, salt, solvate, or polymorph thereof. In an even further aspect, the nucleoside reverse transcriptase inhibitor is didansine, or a pharmaceutically acceptable prodrug, salt, solvate, or polymorph thereof. In a still further aspect, the nucleoside reverse transcriptase inhibitor is elvucitabine, or a pharmaceutically acceptable prodrug, salt, solvate, or polymorph thereof. In yet a further aspect, the nucleoside reverse transcriptase inhibitor is emtricitabine, or a pharmaceutically acceptable prodrug, salt, solvate, or polymorph thereof. In an even further aspect, the nucleoside reverse transcriptase inhibitor is lamivudine, or a pharmaceutically acceptable prodrug, salt, solvate, or polymorph thereof. In a still further aspect, the nucleoside reverse transcriptase inhibitor is stavudine, or a pharmaceutically acceptable prodrug, salt, solvate, or polymorph thereof. In yet a further aspect, the nucleoside reverse transcriptase inhibitor is tenofovir, or a pharmaceutically acceptable prodrug, salt, solvate, or polymorph thereof. In an even further aspect, the nucleoside reverse transcriptase inhibitor is zidovudine, or a pharmaceutically acceptable prodrug, salt, solvate, or polymorph thereof.

In a further aspect, the protease inhibitor is selected from atazanavir, darunavir, fosamprenavir, indinavir, nelfinavir, ritonavir, saquinavir, tipranavir, and lopinavir/ritonavir, or a pharmaceutically acceptable prodrug, salt, solvate, or polymorph thereof. In a still further aspect, the protease inhibitor is atazanavir, or a pharmaceutically acceptable prodrug, salt, solvate, or polymorph thereof. In yet a further aspect, the protease inhibitor is darunavir, or a pharmaceutically acceptable prodrug, salt, solvate, or polymorph thereof. In an even further aspect, the protease inhibitor is fosamprenavir, or a pharmaceutically acceptable prodrug, salt, solvate, or polymorph thereof. In a still further aspect, the protease inhibitor is indinavir, or a pharmaceutically acceptable prodrug, salt, solvate, or polymorph thereof. In yet a further aspect, the protease inhibitor is lopinaviror a pharmaceutically acceptable prodrug, salt, solvate, or polymorph thereof. In an even further aspect, the protease inhibitor is nelfinavir, or a pharmaceutically acceptable prodrug, salt, solvate, or polymorph thereof. In a still further aspect, the protease inhibitor is ritonavir, or a pharmaceutically acceptable prodrug, salt, solvate, or polymorph thereof. In yet a further aspect, the protease inhibitor is saquinavir, or a pharmaceutically acceptable prodrug, salt, solvate, or polymorph thereof. In an even further aspect, the protease inhibitor is tipranavir, or a pharmaceutically acceptable prodrug, salt, solvate, or polymorph thereof. In a still further aspect, the protease inhibitor is lopinavir/ritonavir, or a pharmaceutically acceptable prodrug, salt, solvate, or polymorph thereof.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

### G. METHODS OF TREATING HIV INFECTIONS

Also provided is a method of use of a disclosed compound, composition, or medicament. In one aspect, the method of use is directed to the treatment of a disorder. In a further aspect, the disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein which are usually applied in the treatment of the above mentioned pathological conditions.

1. Treating an HIV Infection by Administering a 1-oxo-2,8-diazaspiro[4.5]decanyl analog

In one aspect, the invention relates to a method for treating a subject for HIV infection, the method comprising the step of administering to the subject an effective amount of a compound having a structure represented by a formula:

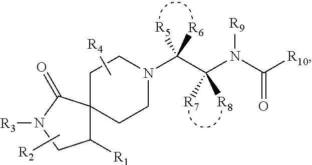

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, trifluoromethyl, carboxamido, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the compound of the method for treating a subject for HIV infection has a structure represented by a formula:

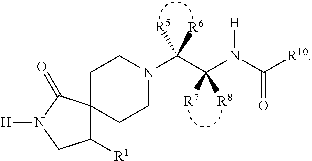

In a further aspect, the subject of the method is mammalian. In a yet further aspect, the subject of the method is human. In a still further aspect, the subject of the method has been diagnosed with a need of treatment for HIV infection prior to the administering step. In an even further aspect, the method further comprises the step of identifying the subject as having a need of treatment for HIV infection.

In a further aspect, the amount of the method is a therapeutically effective amount. In a still further aspect, the amount of the method is a prophylactically effective amount.

In a further aspect, the compound of the method inhibits PLD1 and/or PLD2 response. In a still further aspect, the compound inhibits PLD1 and/or PLD2 activity in an in vitro assay. In a yet further aspect, the compound inhibits PLD1 and/or PLD2 activity in a cell-based assay.

In a further aspect, the compound of the method inhibits PLD1. In a yet further aspect, the compound is a PLD1-selective inhibitor. In a still further aspect, the compound inhibits PLD1 response in an in vitro assay comprising a cultured cell-line. In an even further aspect, the compound inhibits PLD1 response in Calu-1 cells.

In a further aspect, the compound of the method inhibits PLD2. In a yet further aspect, the compound is a PLD2-selective inhibitor. In an even further aspect, the compound inhibits PLD2 response in HEK293gfpPLD2 cells.

In a further aspect, the compound of the method inhibits in vitro PLD1 response. In a yet further aspect, the compound has a PLD1 IC₅₀ of less than about 10 μM, of less than about 1 μM, of less than about 500 nM, of less than about 100 nM, of less than about 60 nM, or of less than about 20 nM. In a still further aspect, the compound exhibits a PLD1:PLD2 inhibition ratio of at least about 2:1, of at least about 3:1, of at least about 5:1, of at least about 10:1, of at least about 20:1, of at least about 50:1, or of at least about 75:1.

In a further aspect, the compound inhibits in vitro PLD2 response. In a yet further aspect, the compound has a PLD2 IC₅₀ of less than about 10 μM, of less than about 1 μM, of less than about 500 nM, of less than about 100 nM, of less than about 60 nM, or of less than about 20 nM. In a still further aspect, the compound exhibits a PLD2:PLD1 inhibition ratio of at least about 2:1, of at least about 3:1, of at least about 5:1, of at least about 10:1, of at least about 20:1, of at least about 50:1, or of at least about 75:1.

In a further aspect, the compound of the method inhibits HIV replication. In a still further aspect, the compound inhibits HIV replication in activated CD4⁺ T-lymphocytes. In yet a further aspect, the compound inhibits HIV replication in tissue macrophages. In an even further aspect, the tissue macrophage is a brain macrophage. In a still further aspect, the tissue macrophage is a microglial cell. In yet a further aspect, the compound inhibits HIV replication in monocytes, dendritic cells, and activated CD4⁺ T-lymphocytes. In an even further aspect, the compound inhibits HIV replication in monocytes. In a still further aspect, the compound inhibits HIV replication in dendritic cells. In a yet further aspect, the compound inhibits HIV replication in activated CD4⁺ T-lymphocytes.

In a further aspect, the compound of the method inhibits HIV integration.

In a further aspect, the HIV infection comprises an HIV-1 serotype virus. In a still further aspect, the HIV-1 infection comprises a Group M, Group N, Group O, or Group P virus strain. In yet a further aspect, the HIV-1 infection comprises a Group M virus strain. In an even further aspect, the HIV-1 Group M virus strain is selected from the subtypes A, B, C, D, F, G, H, J, and K. In a still further aspect, the HIV-1 Group M virus strain subtype is subtype A. In yet a further aspect, the HIV-1 Group M virus strain subtype is subtype B. In an even further aspect, the HIV-1 Group M virus strain subtype is subtype C. In a still further aspect, the HIV-1 Group M virus strain subtype is subtype D. In yet a further aspect, the HIV-1 Group M virus strain subtype is subtype H. In an even further aspect, the HIV-1 Group M virus strain subtype comprises a circulating recombinant form (“CRF”) comprising genetic material from one or more subtypes selected from subtypes A, B, C, D, F, G, H, J, and K. In a still further aspect, the circulating recombinant form is CRF A/E. In yet a further aspect, the circulating recombinant form is CRF A/G.

In a further aspect, the HIV infection of the method comprises an HIV-2 serotype virus.

In a further aspect, the HIV infection of the method is associated with a disease selected from AIDS, aspergillosis, atypical mycobacteriosis, bacillary angiomatosis, bacteremia, bacterial pneumonia, bacterial sinusitis, candidiasis, CMV, CMV retinitis, coccidioidomycosis, cryptococcosis, cryptosporidiosis-isosporiasis, non-specific enteritis, folliculitis, herpes, histoplasmosis, HIV dementia, HIV meningitis, leismaniasis, Mycobacterium avium complex disease, nocardiosis, pencilliosis, progressive multifocal leukoencephalopathy (PML; or HIV encephalitis), Pneumocystis carinii pneumonia (PCP), pneumonia, Pseudomonas pneumonia, toxoplasma encephalitis, toxoplasmosis, tuberculosis, Kaposi sarcoma, lymphoma, and squamous cell carcinoma. In yet a further aspect, the lymphoma is selected from Non-Hodgkin's lymphoma, CNS lymphoma, primary lymphoma of the brain, and systemic lymphoma.

In a further aspect, the HIV infection of the method is associated with a cancer. In a still further aspect, the cancer is selected from a lymphoma, sarcoma, and a carcinoma. In yet a further aspect, the carcinoma is a squamous cell carcinoma. In an even further aspect, the sarcoma is Kaposi sarcoma. In a still further aspect, the lymphoma is selected from Non-Hodgkin's lymphoma, CNS lymphoma, primary lymphoma of the brain, and systemic lymphoma.

In a further aspect, the HIV infection of the method is associated with an opportunistic infection. In a still further aspect, the opportunistic infection is selected from aspergillosis, atypical mycobacteriosis, bacillary angiomatosis, bacteremia, bacterial pneumonia, bacterial sinusitis, candidiasis, CMV retinitis, coccidioidomycosis, cryptococcosis, cryptosporidiosis-isosporiasis, non-specific enteritis, folliculitis, herpes, histoplasmosis, HIV dementia, HIV meningitis, leismaniasis, Mycobacterium avium complex disease, nocardiosis, pencilliosis, progressive multifocal leukoencephalopathy (PML; or HIV encephalitis), Pneumocystis carinii pneumonia (PCP), pneumonia, Pseudomonas pneumonia, toxoplasma encephalitis, toxoplasmosis, and tuberculosis.

In a further aspect, the HIV infection of the method is associated with an infection associated with Cryptosporidium muris, Isospora belli, Toxoplasma gondii, Candida sp., Coccidioides immitis, Histoplasma capsulatum, Pneumocystis carnii, Mycobacterium avium complex, Mycobacterium tuberculosis, Cytomegalovirus, Epstein-Barr virus, Herpes simplex virus, Papovirus J-C, or Varicella-zoster.

In a further aspect, the HIV infection of the method comprises an HIV virus that is resistant to treatment with a non-nucleoside reverse transcriptase inhibitor. In a still further aspect, the HIV virus resistant to treatment with a non-nucleoside reverse transcriptase inhibitor has at least one mutation in the HIV reverse transcriptase. In yet a further aspect, the at least one mutation in the HIV reverse transcriptase is selected from 100I, 103N, 106A, 106M, 108I, 181C, 181I, 188C, 188H, 188L, 190A, 190S, 225H, 230L, and 236L. In an even further aspect, the at least one mutation is at amino acid position 100, 103, 106, 108, 181, 188, 190, 225, 230, or 236 of the HIV reverse transcriptase. In a still further aspect, the non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In yet a further aspect, the non-nucleoside reverse transcriptase inhibitor is delavirdine, or a pharmaceutically acceptable prodrug, salt, solvate, or polymorph thereof. In an even further aspect, the non-nucleoside reverse transcriptase inhibitor is efavirenz, or a pharmaceutically acceptable prodrug, salt, solvate, or polymorph thereof. In a still further aspect, the non-nucleoside reverse transcriptase inhibitor is etravirine, or a pharmaceutically acceptable prodrug, salt, solvate, or polymorph thereof. In yet a further aspect, the non-nucleoside reverse transcriptase inhibitor is nevirapine, or a pharmaceutically acceptable prodrug, salt, solvate, or polymorph thereof. In an even further aspect, the non-nucleoside reverse transcriptase inhibitor is rilpivirine, or a pharmaceutically acceptable prodrug, salt, solvate, or polymorph thereof. In a still further aspect, the non-nucleoside reverse transcriptase inhibitor is lersivirine, or a pharmaceutically acceptable prodrug, salt, solvate, or polymorph thereof.

In a further aspect, the HIV infection of the method comprises an HIV virus that is resistant to treatment with a nucleoside reverse transcriptase inhibitor. In a still further aspect, the HIV virus resistant to treatment with a nucleoside reverse transcriptase inhibitor has at least one mutation in the HIV reverse transcriptase. In yet a further aspect, the at least one mutation in the HIV reverse transcriptase is selected from 41L, 44D, 62V, 65R, 67N, 69A, 69D, 69N, 69S, 69 insertion, 70R, 74V, 75I, 77L, 115F, 116Y, 118I, 151M, 184I, 184V, 210W, 215C, 215D, 215E, 215F, 215I, 215S, 215Y, 219E, and 219Q. In an even further aspect, the at least one mutation is at amino acid position 41, 44, 62, 65, 67, 69, 70, 74, 77, 115, 116, 118, 151, 184, 210, 215 or 219 of the HIV reverse transcriptase. In a still further aspect, the nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In yet a further aspect, the nucleoside reverse transcriptase inhibitor is selected from abacavir, didansine, emtricitabine, lamivudine, stavudine, tenofovir, zidovudine, and elvucitabine, or a pharmaceutically acceptable prodrug, salt, solvate, or polymorph thereof. In an even further aspect, the nucleoside reverse transcriptase inhibitor is abacavir, or a pharmaceutically acceptable prodrug, salt, solvate, or polymorph thereof. In a still further aspect, the nucleoside reverse transcriptase inhibitor is didansine, or a pharmaceutically acceptable prodrug, salt, solvate, or polymorph thereof. In yet a further aspect, the nucleoside reverse transcriptase inhibitor is emtricitabine, or a pharmaceutically acceptable prodrug, salt, solvate, or polymorph thereof. In an even further aspect, the nucleoside reverse transcriptase inhibitor is lamivudine, or a pharmaceutically acceptable prodrug, salt, solvate, or polymorph thereof. In a still further aspect, the nucleoside reverse transcriptase inhibitor is stavudine, or a pharmaceutically acceptable prodrug, salt, solvate, or polymorph thereof. In yet a further aspect, the nucleoside reverse transcriptase inhibitor is tenofovir, or a pharmaceutically acceptable prodrug, salt, solvate, or polymorph thereof. In an even further aspect, the nucleoside reverse transcriptase inhibitor is zidovudine, or a pharmaceutically acceptable prodrug, salt, solvate, or polymorph thereof. In a still further aspect, the nucleoside reverse transcriptase inhibitor is elvucitabine, or a pharmaceutically acceptable prodrug, salt, solvate, or polymorph thereof.

In a further aspect, the HIV infection of the method comprises an HIV virus that is resistant to treatment with a protease inhibitor. In a still further aspect, the HIV virus resistant to treatment with a protease inhibitor has at least one mutation in the HIV protease. In yet a further aspect, the at least one mutation in the HIV protease is selected from 30N, 46I, 46L, 48V, 50V, 82A, 82F, 82S, 82T, 84V, and 90M. In an even further aspect, the at least one mutation is at amino acid position 30, 46, 48, 50, 82, 84, or 90 of the HIV protease. In a still further aspect, the protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In yet a further aspect, the protease inhibitor is atazanavir, or a pharmaceutically acceptable prodrug, salt, solvate, or polymorph thereof. In an even further aspect, the protease inhibitor is darunavir, or a pharmaceutically acceptable prodrug, salt, solvate, or polymorph thereof. In a still further aspect, the protease inhibitor is fosamprenavir, or a pharmaceutically acceptable prodrug, salt, solvate, or polymorph thereof. In yet a further aspect, the protease inhibitor is indinavir, or a pharmaceutically acceptable prodrug, salt, solvate, or polymorph thereof. In an even further aspect, the protease inhibitor is lopinavir, or a pharmaceutically acceptable prodrug, salt, solvate, or polymorph thereof. In a still further aspect, the protease inhibitor is nelfinavir, or a pharmaceutically acceptable prodrug, salt, solvate, or polymorph thereof. In yet a further aspect, the protease inhibitor is ritonavir, or a pharmaceutically acceptable prodrug, salt, solvate, or polymorph thereof. In an even further aspect, the protease inhibitor is saquinavir, or a pharmaceutically acceptable prodrug, salt, solvate, or polymorph thereof. In a still further aspect, the protease inhibitor is tipranavir, or a pharmaceutically acceptable prodrug, salt, solvate, or polymorph thereof. In yet a further aspect, the protease inhibitor is lopinavir/ritonavir, or a pharmaceutically acceptable prodrug, salt, solvate, or polymorph thereof.

In a further aspect, the HIV infection of the method comprises an HIV virus that is resistant to treatment with an integrase inhibitor. In a still further aspect, the integrase inhibitor is selected from raltegravir, dolutegravir, elvitegravir, and S/GSK1265744, or a pharmaceutically acceptable prodrug, salt, solvate, or polymorph thereof. In yet a further aspect, the integrase inhibitor is selected from raltegravir, dolutegravir, and elvitegravir, or a pharmaceutically acceptable prodrug, salt, solvate, or polymorph thereof. In an even further aspect, the integrase inhibitor is raltegravir, or a pharmaceutically acceptable prodrug, salt, solvate, or polymorph thereof. In a still further aspect, the integrase inhibitor is dolutegravir, or a pharmaceutically acceptable prodrug, salt, solvate, or polymorph thereof. In yet a further aspect, the integrase inhibitor is elvitegravir, or a pharmaceutically acceptable prodrug, salt, solvate, or polymorph thereof.

In a further aspect, the HIV infection of the method comprises an HIV virus that is resistant to treatment with a fusion inhibitor. In a still further aspect, the fusion inhibitor is selected from enfuvirtide, maraviroc, cenicriviroc, ibalizumab, BMS-663068, and PRO-140, or a pharmaceutically acceptable prodrug, salt, solvate, or polymorph thereof. In yet a further aspect, the fusion inhibitor is selected from enfuvirtide, maraviroc, cenicriviroc, and ibalizumab, or a pharmaceutically acceptable prodrug, salt, solvate, or polymorph thereof. In an even further aspect, the fusion inhibitor is enfuvirtide, or a pharmaceutically acceptable prodrug, salt, solvate, or polymorph thereof. In a still further aspect, the fusion inhibitor is maraviroc, or a pharmaceutically acceptable prodrug, salt, solvate, or polymorph thereof. In yet a further aspect, the fusion inhibitor is cenicriviroc, or a pharmaceutically acceptable prodrug, salt, solvate, or polymorph thereof. In an even further aspect, the fusion inhibitor is ibalizumab, or a pharmaceutically acceptable prodrug, salt, solvate, or polymorph thereof.

In a further aspect, the method further comprises assessing viral load in the subject following administration.

In a further aspect, the method further comprises administering to the subject a non-PLD anti-HIV therapy.

In a further aspect, the administering of the method comprises inhalation or oral administration. In a still further aspect, the administering of the method comprises intravenous or intra-arterial injection.

2. Treating an HIV Infection by Administering a 4-oxo-1,3,8-triazaspiro[4.5]decanyl analog

In various aspects, the invention relates to a method for treating a subject for HIV infection, the method comprising the step of administering to the subject an effective amount of a compound having a structure represented by a formula:

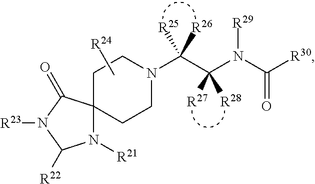

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, trifluoromethyl, carboxamido, alkylsulfonyl, an optionally substituted C1 to C6 alkyl, or an optionally substituted C3 to C6 cycloalkyl or R²⁵ and R²⁶, together with the intermediate carbon, comprise an optionally substituted C3 to C6 cycloalkyl; wherein each of R²⁷ and R²⁸ independently comprises hydrogen, trifluoromethyl, carboxamido, alkylsulfonyl, an optionally substituted C1 to C6 alkyl, or an optionally substituted C3 to C6 cycloalkyl or R²⁷ and R²⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the compound of the method for treating a subject for HIV infection has a structure represented by a formula:

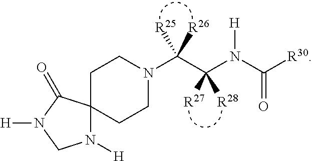

In a further aspect, the compound of the method for treating a subject for HIV infection is:

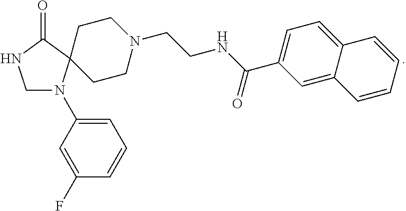 [SMILES: O=C(NCCN1CCC2(CC1)C(=O)NCN2c1cccc(F)c1)c1ccc2ccccc2c1]

In a further aspect, the subject of the method is mammalian. In a yet further aspect, the subject of the method is human. In a still further aspect, the subject of the method has been diagnosed with a need of treatment for HIV infection prior to the administering step. In an even further aspect, the method further comprises the step of identifying the subject as having a need of treatment for HIV infection.

In a further aspect, the amount of the method is a therapeutically effective amount. In a still further aspect, the amount of the method is a prophylactically effective amount.

In a further aspect, the compound of the method inhibits PLD1 and/or PLD2 response. In a still further aspect, the compound inhibits PLD1 and/or PLD2 activity in an in vitro assay. In a yet further aspect, the compound inhibits PLD1 and/or PLD2 activity in a cell-based assay.

In a further aspect, the compound of the method inhibits PLD1. In a yet further aspect, the compound is a PLD1-selective inhibitor. In a still further aspect, the compound inhibits PLD1 response in an in vitro assay comprising a cultured cell-line. In an even further aspect, the compound inhibits PLD1 response in Calu-1 cells.

In a further aspect, the compound of the method inhibits PLD2. In a yet further aspect, the compound is a PLD2-selective inhibitor. In an even further aspect, the compound inhibits PLD2 response in HEK293gfpPLD2 cells.

In a further aspect, the compound of the method inhibits in vitro PLD1 response. In a yet further aspect, the compound has a PLD1 IC₅₀ of less than about 10 μM, of less than about 1 μM, of less than about 500 nM, of less than about 100 nM, of less than about 60 nM, or of less than about 20 nM. In a still further aspect, the compound exhibits a PLD1:PLD2 inhibition ratio of at least about 2:1, of at least about 3:1, of at least about 5:1, of at least about 10:1, of at least about 20:1, of at least about 50:1, or of at least about 75:1.

In a further aspect, the compound inhibits in vitro PLD2 response. In a yet further aspect, the compound has a PLD2 IC₅₀ of less than about 10 μM, of less than about 1 μM, of less than about 500 nM, of less than about 100 nM, of less than about 60 nM, or of less than about 20 nM. In a still further aspect, the compound exhibits a PLD2:PLD1 inhibition ratio of at least about 2:1, of at least about 3:1, of at least about 5:1, of at least about 10:1, of at least about 20:1, of at least about 50:1, or of at least about 75:1.

In a further aspect, the compound of the method inhibits HIV replication. In a still further aspect, the compound inhibits HIV replication in activated CD4⁺ T-lymphocytes. In yet a further aspect, the compound inhibits HIV replication in tissue macrophages. In an even further aspect, the tissue macrophage is a brain macrophage. In a still further aspect, the tissue macrophage is a microglial cell. In yet a further aspect, the compound inhibits HIV replication in monocytes, dendritic cells, and activated CD4⁺ T-lymphocytes. In an even further aspect, the compound inhibits HIV replication in monocytes. In a still further aspect, the compound inhibits HIV replication in dendritic cells. In a yet further aspect, the compound inhibits HIV replication in activated CD4⁺ T-lymphocytes.

In a further aspect, the compound of the method inhibits HIV integration.

In a further aspect, the HIV infection comprises an HIV-1 serotype virus. In a still further aspect, the HIV-1 infection comprises a Group M, Group N, Group O, or Group P virus strain. In yet a further aspect, the HIV-1 infection comprises a Group M virus strain. In an even further aspect, the HIV-1 Group M virus strain is selected from the subtypes A, B, C, D, F, G, H, J, and K. In a still further aspect, the HIV-1 Group M virus strain subtype is subtype A. In yet a further aspect, the HIV-1 Group M virus strain subtype is subtype B. In an even further aspect, the HIV-1 Group M virus strain subtype is subtype C. In a still further aspect, the HIV-1 Group M virus strain subtype is subtype D. In yet a further aspect, the HIV-1 Group M virus strain subtype is subtype H. In an even further aspect, the HIV-1 Group M virus strain subtype comprises a circulating recombinant form (“CRF”) comprising genetic material from one or more subtypes selected from subtypes A, B, C, D, F, G, H, J, and K. In a still further aspect, the circulating recombinant form is CRF A/E. In yet a further aspect, the circulating recombinant form is CRF A/G.

In a further aspect, the HIV infection of the method comprises an HIV-2 serotype virus.

In a further aspect, the HIV infection of the method is associated with a disease selected from AIDS, aspergillosis, atypical mycobacteriosis, bacillary angiomatosis, bacteremia, bacterial pneumonia, bacterial sinusitis, candidiasis, CMV, CMV retinitis, coccidioidomycosis, cryptococcosis, cryptosporidiosis-isosporiasis, non-specific enteritis, folliculitis, herpes, histoplasmosis, HIV dementia, HIV meningitis, leismaniasis, Mycobacterium avium complex disease, nocardiosis, pencilliosis, progressive multifocal leukoencephalopathy (PML; or HIV encephalitis), Pneumocystis carinii pneumonia (PCP), pneumonia, Pseudomonas pneumonia, toxoplasma encephalitis, toxoplasmosis, tuberculosis, Kaposi sarcoma, lymphoma, and squamous cell carcinoma. In yet a further aspect, the lymphoma is selected from Non-Hodgkin's lymphoma, CNS lymphoma, primary lymphoma of the brain, and systemic lymphoma.

In a further aspect, the HIV infection of the method is associated with a cancer. In a still further aspect, the cancer is selected from a lymphoma, sarcoma, and a carcinoma. In yet a further aspect, the carcinoma is a squamous cell carcinoma. In an even further aspect, the sarcoma is Kaposi sarcoma. In a still further aspect, the lymphoma is selected from Non-Hodgkin's lymphoma, CNS lymphoma, primary lymphoma of the brain, and systemic lymphoma.

In a further aspect, the HIV infection of the method is associated with an opportunistic infection. In a still further aspect, the opportunistic infection is selected from aspergillosis, atypical mycobacteriosis, bacillary angiomatosis, bacteremia, bacterial pneumonia, bacterial sinusitis, candidiasis, CMV retinitis, coccidioidomycosis, cryptococcosis, cryptosporidiosis-isosporiasis, non-specific enteritis, folliculitis, herpes, histoplasmosis, HIV dementia, HIV meningitis, leismaniasis, Mycobacterium avium complex disease, nocardiosis, pencilliosis, progressive multifocal leukoencephalopathy (PML; or HIV encephalitis), Pneumocystis carinii pneumonia (PCP), pneumonia, Pseudomonas pneumonia, toxoplasma encephalitis, toxoplasmosis, and tuberculosis.

In a further aspect, the HIV infection of the method is associated with an infection associated with Cryptosporidium muris, Isospora belli, Toxoplasma gondii, Candida sp., Coccidioides immitis, Histoplasma capsulatum, Pneumocystis carnii, Mycobacterium avium complex, Mycobacterium tuberculosis, Cytomegalovirus, Epstein-Barr virus, Herpes simplex virus, Papovirus J-C, or Varicella-zoster.

In a further aspect, the HIV infection of the method comprises an HIV virus that is resistant to treatment with a non-nucleoside reverse transcriptase inhibitor. In a still further aspect, the HIV virus resistant to treatment with a non-nucleoside reverse transcriptase inhibitor has at least one mutation in the HIV reverse transcriptase. In yet a further aspect, the at least one mutation in the HIV reverse transcriptase is selected from 100I, 103N, 106A, 106M, 108I, 181C, 181I, 188C, 188H, 188L, 190A, 190S, 225H, 230L, and 236L. In an even further aspect, the at least one mutation is at amino acid position 100, 103, 106, 108, 181, 188, 190, 225, 230, or 236 of the HIV reverse transcriptase. In a still further aspect, the non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In yet a further aspect, the non-nucleoside reverse transcriptase inhibitor is delavirdine, or a pharmaceutically acceptable prodrug, salt, solvate, or polymorph thereof. In an even further aspect, the non-nucleoside reverse transcriptase inhibitor is efavirenz, or a pharmaceutically acceptable prodrug, salt, solvate, or polymorph thereof. In a still further aspect, the non-nucleoside reverse transcriptase inhibitor is etravirine, or a pharmaceutically acceptable prodrug, salt, solvate, or polymorph thereof. In yet a further aspect, the non-nucleoside reverse transcriptase inhibitor is nevirapine, or a pharmaceutically acceptable prodrug, salt, solvate, or polymorph thereof. In an even further aspect, the non-nucleoside reverse transcriptase inhibitor is rilpivirine, or a pharmaceutically acceptable prodrug, salt, solvate, or polymorph thereof. In a still further aspect, the non-nucleoside reverse transcriptase inhibitor is lersivirine, or a pharmaceutically acceptable prodrug, salt, solvate, or polymorph thereof.

In a further aspect, the HIV infection of the method comprises an HIV virus that is resistant to treatment with a nucleoside reverse transcriptase inhibitor. In a still further aspect, the HIV virus resistant to treatment with a nucleoside reverse transcriptase inhibitor has at least one mutation in the HIV reverse transcriptase. In yet a further aspect, the at least one mutation in the HIV reverse transcriptase is selected from 41L, 44D, 62V, 65R, 67N, 69A, 69D, 69N, 69S, 69 insertion, 70R, 74V, 75I, 77L, 115F, 116Y, 118I, 151M, 184I, 184V, 210W, 215C, 215D, 215E, 215F, 215I, 215S, 215Y, 219E, and 219Q. In an even further aspect, the at least one mutation is at amino acid position 41, 44, 62, 65, 67, 69, 70, 74, 77, 115, 116, 118, 151, 184, 210, 215 or 219 of the HIV reverse transcriptase. In a still further aspect, the nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In yet a further aspect, the nucleoside reverse transcriptase inhibitor is selected from abacavir, didansine, emtricitabine, lamivudine, stavudine, tenofovir, zidovudine, and elvucitabine, or a pharmaceutically acceptable prodrug, salt, solvate, or polymorph thereof. In an even further aspect, the nucleoside reverse transcriptase inhibitor is abacavir, or a pharmaceutically acceptable prodrug, salt, solvate, or polymorph thereof. In a still further aspect, the nucleoside reverse transcriptase inhibitor is didansine, or a pharmaceutically acceptable prodrug, salt, solvate, or polymorph thereof. In yet a further aspect, the nucleoside reverse transcriptase inhibitor is emtricitabine, or a pharmaceutically acceptable prodrug, salt, solvate, or polymorph thereof. In an even further aspect, the nucleoside reverse transcriptase inhibitor is lamivudine, or a pharmaceutically acceptable prodrug, salt, solvate, or polymorph thereof. In a still further aspect, the nucleoside reverse transcriptase inhibitor is stavudine, or a pharmaceutically acceptable prodrug, salt, solvate, or polymorph thereof. In yet a further aspect, the nucleoside reverse transcriptase inhibitor is tenofovir, or a pharmaceutically acceptable prodrug, salt, solvate, or polymorph thereof. In an even further aspect, the nucleoside reverse transcriptase inhibitor is zidovudine, or a pharmaceutically acceptable prodrug, salt, solvate, or polymorph thereof. In a still further aspect, the nucleoside reverse transcriptase inhibitor is elvucitabine, or a pharmaceutically acceptable prodrug, salt, solvate, or polymorph thereof.

In a further aspect, the HIV infection of the method comprises an HIV virus that is resistant to treatment with a protease inhibitor. In a still further aspect, the HIV virus resistant to treatment with a protease inhibitor has at least one mutation in the HIV protease. In yet a further aspect, the at least one mutation in the HIV protease is selected from 30N, 46I, 46L, 48V, 50V, 82A, 82F, 82S, 82T, 84V, and 90M. In an even further aspect, the at least one mutation is at amino acid position 30, 46, 48, 50, 82, 84, or 90 of the HIV protease. In a still further aspect, the protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In yet a further aspect, the protease inhibitor is atazanavir, or a pharmaceutically acceptable prodrug, salt, solvate, or polymorph thereof. In an even further aspect, the protease inhibitor is darunavir, or a pharmaceutically acceptable prodrug, salt, solvate, or polymorph thereof. In a still further aspect, the protease inhibitor is fosamprenavir, or a pharmaceutically acceptable prodrug, salt, solvate, or polymorph thereof. In yet a further aspect, the protease inhibitor is indinavir, or a pharmaceutically acceptable prodrug, salt, solvate, or polymorph thereof. In an even further aspect, the protease inhibitor is lopinavir, or a pharmaceutically acceptable prodrug, salt, solvate, or polymorph thereof. In a still further aspect, the protease inhibitor is nelfinavir, or a pharmaceutically acceptable prodrug, salt, solvate, or polymorph thereof. In yet a further aspect, the protease inhibitor is ritonavir, or a pharmaceutically acceptable prodrug, salt, solvate, or polymorph thereof. In an even further aspect, the protease inhibitor is saquinavir, or a pharmaceutically acceptable prodrug, salt, solvate, or polymorph thereof. In a still further aspect, the protease inhibitor is tipranavir, or a pharmaceutically acceptable prodrug, salt, solvate, or polymorph thereof. In yet a further aspect, the protease inhibitor is lopinavir/ritonavir, or a pharmaceutically acceptable prodrug, salt, solvate, or polymorph thereof.

In a further aspect, the HIV infection of the method comprises an HIV virus that is resistant to treatment with an integrase inhibitor. In a still further aspect, the integrase inhibitor is selected from raltegravir, dolutegravir, elvitegravir, and S/GSK1265744, or a pharmaceutically acceptable prodrug, salt, solvate, or polymorph thereof. In yet a further aspect, the integrase inhibitor is selected from raltegravir, dolutegravir, and elvitegravir, or a pharmaceutically acceptable prodrug, salt, solvate, or polymorph thereof. In an even further aspect, the integrase inhibitor is raltegravir, or a pharmaceutically acceptable prodrug, salt, solvate, or polymorph thereof. In a still further aspect, the integrase inhibitor is dolutegravir, or a pharmaceutically acceptable prodrug, salt, solvate, or polymorph thereof. In yet a further aspect, the integrase inhibitor is elvitegravir, or a pharmaceutically acceptable prodrug, salt, solvate, or polymorph thereof.

In a further aspect, the HIV infection of the method comprises an HIV virus that is resistant to treatment with a fusion inhibitor. In a still further aspect, the fusion inhibitor is selected from enfuvirtide, maraviroc, cenicriviroc, ibalizumab, BMS-663068, and PRO-140, or a pharmaceutically acceptable prodrug, salt, solvate, or polymorph thereof. In yet a further aspect, the fusion inhibitor is selected from enfuvirtide, maraviroc, cenicriviroc, and ibalizumab, or a pharmaceutically acceptable prodrug, salt, solvate, or polymorph thereof. In an even further aspect, the fusion inhibitor is enfuvirtide, or a pharmaceutically acceptable prodrug, salt, solvate, or polymorph thereof. In a still further aspect, the fusion inhibitor is maraviroc, or a pharmaceutically acceptable prodrug, salt, solvate, or polymorph thereof. In yet a further aspect, the fusion inhibitor is cenicriviroc, or a pharmaceutically acceptable prodrug, salt, solvate, or polymorph thereof. In an even further aspect, the fusion inhibitor is ibalizumab, or a pharmaceutically acceptable prodrug, salt, solvate, or polymorph thereof.

In a further aspect, the method further comprises assessing viral load in the subject following administration.

In a further aspect, the method further comprises administering to the subject a non-PLD anti-HIV therapy.

In a further aspect, the administering of the method comprises inhalation or oral administration. In a still further aspect, the administering of the method comprises intravenous or intra-arterial injection.

3. Treating an HIV Infection by Administering a Substituted 2-oxo-2,3-dihydro-1H-benzo[d]imidazol-1-yl analog

In one aspect, the invention relates to a method for treating a subject for HIV infection, the method comprising the step of administering to the subject an effective amount of a compound having a structure represented by a formula:

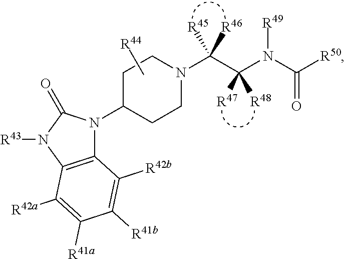

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, trifluoromethyl, carboxamido, alkylsulfonyl, an optionally substituted C1 to C6 alkyl, or an optionally substituted C3 to C6 cycloalkyl or R⁴⁵ and R⁴⁶, together with the intermediate carbon, comprise an optionally substituted C3 to C6 cycloalkyl; wherein each of R⁴⁷ and R⁴⁸ independently comprises hydrogen, trifluoromethyl, carboxamido, alkylsulfonyl, an optionally substituted C1 to C6 alkyl, or an optionally substituted C3 to C6 cycloalkyl or R⁴⁷ and R⁴⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the compound of the method for treating a subject for HIV infection has a structure represented by a formula:

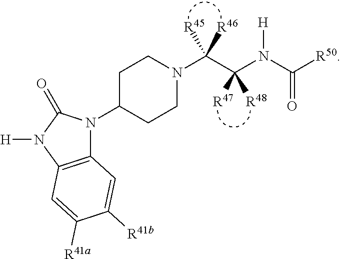

In a further aspect, the compound of the method for treating a subject for HIV infection is:

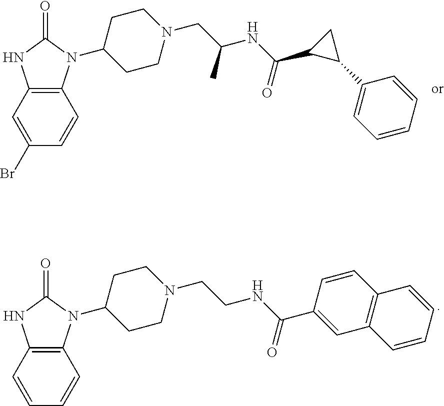

In a further aspect, the subject of the method is mammalian. In a yet further aspect, the subject of the method is human. In a still further aspect, the subject of the method has been diagnosed with a need of treatment for HIV infection prior to the administering step. In an even further aspect, the method further comprises the step of identifying the subject as having a need of treatment for HIV infection.

In a further aspect, the amount of the method is a therapeutically effective amount. In a still further aspect, the amount of the method is a prophylactically effective amount.

In a further aspect, the compound of the method inhibits PLD1 and/or PLD2 response. In a still further aspect, the compound inhibits PLD1 and/or PLD2 activity in an in vitro assay. In a yet further aspect, the compound inhibits PLD1 and/or PLD2 activity in a cell-based assay.

In a further aspect, the compound of the method inhibits PLD1. In a yet further aspect, the compound is a PLD1-selective inhibitor. In a still further aspect, the compound inhibits PLD1 response in an in vitro assay comprising a cultured cell-line. In an even further aspect, the compound inhibits PLD1 response in Calu-1 cells.

In a further aspect, the compound of the method inhibits PLD2. In a yet further aspect, the compound is a PLD2-selective inhibitor. In an even further aspect, the compound inhibits PLD2 response in HEK293gfpPLD2 cells.

In a further aspect, the compound of the method inhibits in vitro PLD1 response. In a yet further aspect, the compound has a PLD1 IC₅₀ of less than about 10 μM, of less than about 1 μM, of less than about 500 nM, of less than about 100 nM, of less than about 60 nM, or of less than about 20 nM. In a still further aspect, the compound exhibits a PLD1:PLD2 inhibition ratio of at least about 2:1, of at least about 3:1, of at least about 5:1, of at least about 10:1, of at least about 20:1, of at least about 50:1, or of at least about 75:1.

In a further aspect, the compound inhibits in vitro PLD2 response. In a yet further aspect, the compound has a PLD2 IC₅₀ of less than about 10 μM, of less than about 1 μM, of less than about 500 nM, of less than about 100 nM, of less than about 60 nM, or of less than about 20 nM. In a still further aspect, the compound exhibits a PLD2:PLD1 inhibition ratio of at least about 2:1, of at least about 3:1, of at least about 5:1, of at least about 10:1, of at least about 20:1, of at least about 50:1, or of at least about 75:1.

In a further aspect, the compound of the method inhibits HIV replication. In a still further aspect, the compound inhibits HIV replication in activated CD4⁺ T-lymphocytes. In yet a further aspect, the compound inhibits HIV replication in tissue macrophages. In an even further aspect, the tissue macrophage is a brain macrophage. In a still further aspect, the tissue macrophage is a microglial cell. In yet a further aspect, the compound inhibits HIV replication in monocytes, dendritic cells, and activated CD4⁺ T-lymphocytes. In an even further aspect, the compound inhibits HIV replication in monocytes. In a still further aspect, the compound inhibits HIV replication in dendritic cells. In a yet further aspect, the compound inhibits HIV replication in activated CD4⁺ T-lymphocytes.

In a further aspect, the compound of the method inhibits HIV integration.

In a further aspect, the HIV infection comprises an HIV-1 serotype virus. In a still further aspect, the HIV-1 infection comprises a Group M, Group N, Group O, or Group P virus strain. In yet a further aspect, the HIV-1 infection comprises a Group M virus strain. In an even further aspect, the HIV-1 Group M virus strain is selected from the subtypes A, B, C, D, F, G, H, J, and K. In a still further aspect, the HIV-1 Group M virus strain subtype is subtype A. In yet a further aspect, the HIV-1 Group M virus strain subtype is subtype B. In an even further aspect, the HIV-1 Group M virus strain subtype is subtype C. In a still further aspect, the HIV-1 Group M virus strain subtype is subtype D. In yet a further aspect, the HIV-1 Group M virus strain subtype is subtype H. In an even further aspect, the HIV-1 Group M virus strain subtype comprises a circulating recombinant form (“CRF”) comprising genetic material from one or more subtypes selected from subtypes A, B, C, D, F, G, H, J, and K. In a still further aspect, the circulating recombinant form is CRF A/E. In yet a further aspect, the circulating recombinant form is CRF A/G.

In a further aspect, the HIV infection of the method comprises an HIV-2 serotype virus.

In a further aspect, the HIV infection of the method is associated with a disease selected from AIDS, aspergillosis, atypical mycobacteriosis, bacillary angiomatosis, bacteremia, bacterial pneumonia, bacterial sinusitis, candidiasis, CMV, CMV retinitis, coccidioidomycosis, cryptococcosis, cryptosporidiosis-isosporiasis, non-specific enteritis, folliculitis, herpes, histoplasmosis, HIV dementia, HIV meningitis, leismaniasis, Mycobacterium avium complex disease, nocardiosis, pencilliosis, progressive multifocal leukoencephalopathy (PML; or HIV encephalitis), Pneumocystis carinii pneumonia (PCP), pneumonia, Pseudomonas pneumonia, toxoplasma encephalitis, toxoplasmosis, tuberculosis, Kaposi sarcoma, lymphoma, and squamous cell carcinoma. In yet a further aspect, the lymphoma is selected from Non-Hodgkin's lymphoma, CNS lymphoma, primary lymphoma of the brain, and systemic lymphoma.

In a further aspect, the HIV infection of the method is associated with a cancer. In a still further aspect, the cancer is selected from a lymphoma, sarcoma, and a carcinoma. In yet a further aspect, the carcinoma is a squamous cell carcinoma. In an even further aspect, the sarcoma is Kaposi sarcoma. In a still further aspect, the lymphoma is selected from Non-Hodgkin's lymphoma, CNS lymphoma, primary lymphoma of the brain, and systemic lymphoma.

In a further aspect, the HIV infection of the method is associated with an opportunistic infection. In a still further aspect, the opportunistic infection is selected from aspergillosis, atypical mycobacteriosis, bacillary angiomatosis, bacteremia, bacterial pneumonia, bacterial sinusitis, candidiasis, CMV retinitis, coccidioidomycosis, cryptococcosis, cryptosporidiosis-isosporiasis, non-specific enteritis, folliculitis, herpes, histoplasmosis, HIV dementia, HIV meningitis, leismaniasis, Mycobacterium avium complex disease, nocardiosis, pencilliosis, progressive multifocal leukoencephalopathy (PML; or HIV encephalitis), Pneumocystis carinii pneumonia (PCP), pneumonia, Pseudomonas pneumonia, toxoplasma encephalitis, toxoplasmosis, and tuberculosis.

In a further aspect, the HIV infection of the method is associated with an infection associated with Cryptosporidium muris, Isospora belli, Toxoplasma gondii, Candida sp., Coccidioides immitis, Histoplasma capsulatum, Pneumocystis carnii, Mycobacterium avium complex, Mycobacterium tuberculosis, Cytomegalovirus, Epstein-Barr virus, Herpes simplex virus, Papovirus J-C, or Varicella-zoster.

In a further aspect, the HIV infection of the method comprises an HIV virus that is resistant to treatment with a non-nucleoside reverse transcriptase inhibitor. In a still further aspect, the HIV virus resistant to treatment with a non-nucleoside reverse transcriptase inhibitor has at least one mutation in the HIV reverse transcriptase. In yet a further aspect, the at least one mutation in the HIV reverse transcriptase is selected from 100I, 103N, 106A, 106M, 108I, 181C, 181I, 188C, 188H, 188L, 190A, 190S, 225H, 230L, and 236L. In an even further aspect, the at least one mutation is at amino acid position 100, 103, 106, 108, 181, 188, 190, 225, 230, or 236 of the HIV reverse transcriptase. In a still further aspect, the non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In yet a further aspect, the non-nucleoside reverse transcriptase inhibitor is delavirdine, or a pharmaceutically acceptable prodrug, salt, solvate, or polymorph thereof. In an even further aspect, the non-nucleoside reverse transcriptase inhibitor is efavirenz, or a pharmaceutically acceptable prodrug, salt, solvate, or polymorph thereof. In a still further aspect, the non-nucleoside reverse transcriptase inhibitor is etravirine, or a pharmaceutically acceptable prodrug, salt, solvate, or polymorph thereof. In yet a further aspect, the non-nucleoside reverse transcriptase inhibitor is nevirapine, or a pharmaceutically acceptable prodrug, salt, solvate, or polymorph thereof. In an even further aspect, the non-nucleoside reverse transcriptase inhibitor is rilpivirine, or a pharmaceutically acceptable prodrug, salt, solvate, or polymorph thereof. In a still further aspect, the non-nucleoside reverse transcriptase inhibitor is lersivirine, or a pharmaceutically acceptable prodrug, salt, solvate, or polymorph thereof.

In a further aspect, the HIV infection of the method comprises an HIV virus that is resistant to treatment with a nucleoside reverse transcriptase inhibitor. In a still further aspect, the HIV virus resistant to treatment with a nucleoside reverse transcriptase inhibitor has at least one mutation in the HIV reverse transcriptase. In yet a further aspect, the at least one mutation in the HIV reverse transcriptase is selected from 41L, 44D, 62V, 65R, 67N, 69A, 69D, 69N, 69S, 69 insertion, 70R, 74V, 75I, 77L, 115F, 116Y, 118I, 151M, 184I, 184V, 210W, 215C, 215D, 215E, 215F, 215I, 215S, 215Y, 219E, and 219Q. In an even further aspect, the at least one mutation is at amino acid position 41, 44, 62, 65, 67, 69, 70, 74, 77, 115, 116, 118, 151, 184, 210, 215 or 219 of the HIV reverse transcriptase. In a still further aspect, the nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In yet a further aspect, the nucleoside reverse transcriptase inhibitor is selected from abacavir, didansine, emtricitabine, lamivudine, stavudine, tenofovir, zidovudine, and elvucitabine, or a pharmaceutically acceptable prodrug, salt, solvate, or polymorph thereof. In an even further aspect, the nucleoside reverse transcriptase inhibitor is abacavir, or a pharmaceutically acceptable prodrug, salt, solvate, or polymorph thereof. In a still further aspect, the nucleoside reverse transcriptase inhibitor is didansine, or a pharmaceutically acceptable prodrug, salt, solvate, or polymorph thereof. In yet a further aspect, the nucleoside reverse transcriptase inhibitor is emtricitabine, or a pharmaceutically acceptable prodrug, salt, solvate, or polymorph thereof. In an even further aspect, the nucleoside reverse transcriptase inhibitor is lamivudine, or a pharmaceutically acceptable prodrug, salt, solvate, or polymorph thereof. In a still further aspect, the nucleoside reverse transcriptase inhibitor is stavudine, or a pharmaceutically acceptable prodrug, salt, solvate, or polymorph thereof. In yet a further aspect, the nucleoside reverse transcriptase inhibitor is tenofovir, or a pharmaceutically acceptable prodrug, salt, solvate, or polymorph thereof. In an even further aspect, the nucleoside reverse transcriptase inhibitor is zidovudine, or a pharmaceutically acceptable prodrug, salt, solvate, or polymorph thereof. In a still further aspect, the nucleoside reverse transcriptase inhibitor is elvucitabine, or a pharmaceutically acceptable prodrug, salt, solvate, or polymorph thereof.

In a further aspect, the HIV infection of the method comprises an HIV virus that is resistant to treatment with a protease inhibitor. In a still further aspect, the HIV virus resistant to treatment with a protease inhibitor has at least one mutation in the HIV protease. In yet a further aspect, the at least one mutation in the HIV protease is selected from 30N, 46I, 46L, 48V, 50V, 82A, 82F, 82S, 82T, 84V, and 90M. In an even further aspect, the at least one mutation is at amino acid position 30, 46, 48, 50, 82, 84, or 90 of the HIV protease. In a still further aspect, the protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In yet a further aspect, the protease inhibitor is atazanavir, or a pharmaceutically acceptable prodrug, salt, solvate, or polymorph thereof. In an even further aspect, the protease inhibitor is darunavir, or a pharmaceutically acceptable prodrug, salt, solvate, or polymorph thereof. In a still further aspect, the protease inhibitor is fosamprenavir, or a pharmaceutically acceptable prodrug, salt, solvate, or polymorph thereof. In yet a further aspect, the protease inhibitor is indinavir, or a pharmaceutically acceptable prodrug, salt, solvate, or polymorph thereof. In an even further aspect, the protease inhibitor is lopinavir, or a pharmaceutically acceptable prodrug, salt, solvate, or polymorph thereof. In a still further aspect, the protease inhibitor is nelfinavir, or a pharmaceutically acceptable prodrug, salt, solvate, or polymorph thereof. In yet a further aspect, the protease inhibitor is ritonavir, or a pharmaceutically acceptable prodrug, salt, solvate, or polymorph thereof. In an even further aspect, the protease inhibitor is saquinavir, or a pharmaceutically acceptable prodrug, salt, solvate, or polymorph thereof. In a still further aspect, the protease inhibitor is tipranavir, or a pharmaceutically acceptable prodrug, salt, solvate, or polymorph thereof. In yet a further aspect, the protease inhibitor is lopinavir/ritonavir, or a pharmaceutically acceptable prodrug, salt, solvate, or polymorph thereof.

In a further aspect, the HIV infection of the method comprises an HIV virus that is resistant to treatment with an integrase inhibitor. In a still further aspect, the integrase inhibitor is selected from raltegravir, dolutegravir, elvitegravir, and S/GSK1265744, or a pharmaceutically acceptable prodrug, salt, solvate, or polymorph thereof. In yet a further aspect, the integrase inhibitor is selected from raltegravir, dolutegravir, and elvitegravir, or a pharmaceutically acceptable prodrug, salt, solvate, or polymorph thereof. In an even further aspect, the integrase inhibitor is raltegravir, or a pharmaceutically acceptable prodrug, salt, solvate, or polymorph thereof. In a still further aspect, the integrase inhibitor is dolutegravir, or a pharmaceutically acceptable prodrug, salt, solvate, or polymorph thereof. In yet a further aspect, the integrase inhibitor is elvitegravir, or a pharmaceutically acceptable prodrug, salt, solvate, or polymorph thereof.

In a further aspect, the HIV infection of the method comprises an HIV virus that is resistant to treatment with a fusion inhibitor. In a still further aspect, the fusion inhibitor is selected from enfuvirtide, maraviroc, cenicriviroc, ibalizumab, BMS-663068, and PRO-140, or a pharmaceutically acceptable prodrug, salt, solvate, or polymorph thereof. In yet a further aspect, the fusion inhibitor is selected from enfuvirtide, maraviroc, cenicriviroc, and ibalizumab, or a pharmaceutically acceptable prodrug, salt, solvate, or polymorph thereof. In an even further aspect, the fusion inhibitor is enfuvirtide, or a pharmaceutically acceptable prodrug, salt, solvate, or polymorph thereof. In a still further aspect, the fusion inhibitor is maraviroc, or a pharmaceutically acceptable prodrug, salt, solvate, or polymorph thereof. In yet a further aspect, the fusion inhibitor is cenicriviroc, or a pharmaceutically acceptable prodrug, salt, solvate, or polymorph thereof. In an even further aspect, the fusion inhibitor is ibalizumab, or a pharmaceutically acceptable prodrug, salt, solvate, or polymorph thereof.

In a further aspect, the method further comprises assessing viral load in the subject following administration.

In a further aspect, the method further comprises administering to the subject a non-PLD anti-HIV therapy.

In a further aspect, the administering of the method comprises inhalation or oral administration. In a still further aspect, the administering of the method comprises intravenous or intra-arterial injection.

4. Treating an HIV Infection by Administering a Selected Compound

In one aspect, the invention relates to a method for treating a subject for HIV infection, the method comprising the step of administering to the subject an effective amount of a compound selected from: trans-diethylstilbestrol, resveratrol, honokiol, SCH420789, presqualene diphosphate, raloxifene, 4-hydroxytamoxifen, 5-fluoro-2-indoyl des-chlorohalopemide, and halopemide, or a pharmaceutically acceptable salt, hydrate, solvate, or polymorph thereof, thereby treating the subject for HIV infection.

In one aspect, the invention relates to a method for treating a subject for HIV infection, the method comprising the step of administering to the subject an effective amount of a compound selected from:

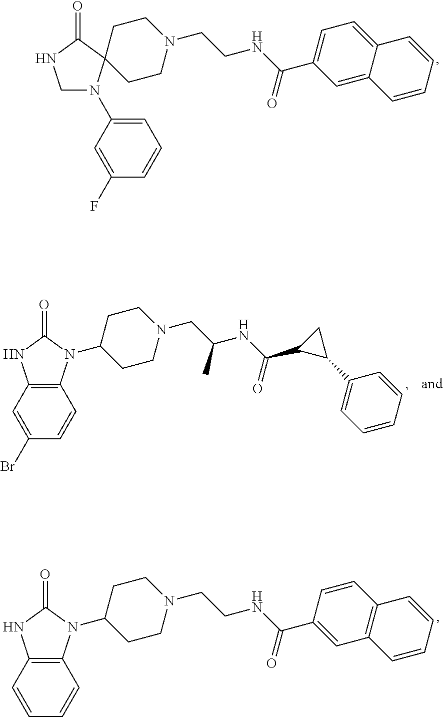

or a pharmaceutically acceptable salt, hydrate, solvate, or polymorph thereof, thereby treating the subject for HIV infection.

In a further aspect, the subject of the method is mammalian. In a yet further aspect, the subject of the method is human. In a still further aspect, the subject of the method has been diagnosed with a need of treatment for HIV infection prior to the administering step. In an even further aspect, the method further comprises the step of identifying the subject as having a need of treatment for HIV infection.

In a further aspect, the effective amount of the method is a therapeutically effective amount. In a still further aspect, the amount of the method is a prophylactically effective amount.

In a further aspect, the compound of the method inhibits PLD1 and/or PLD2 response. In a still further aspect, the compound inhibits PLD1 and/or PLD2 activity in an in vitro assay. In a yet further aspect, the compound inhibits PLD1 and/or PLD2 activity in a cell-based assay.

In a further aspect, the compound of the method inhibits PLD1. In a yet further aspect, the compound is a PLD1-selective inhibitor. In a still further aspect, the compound inhibits PLD1 response in an in vitro assay comprising a cultured cell-line. In an even further aspect, the compound inhibits PLD1 response in Calu-1 cells.

In a further aspect, the compound of the method inhibits PLD2. In a yet further aspect, the compound is a PLD2-selective inhibitor. In an even further aspect, the compound inhibits PLD2 response in HEK293gfpPLD2 cells.

In a further aspect, the compound of the method inhibits in vitro PLD1 response. In a yet further aspect, the compound has a PLD1 IC₅₀ of less than about 10 μM, of less than about 1 μM, of less than about 500 nM, of less than about 100 nM, of less than about 60 nM, or of less than about 20 nM. In a still further aspect, the compound exhibits a PLD1:PLD2 inhibition ratio of at least about 2:1, of at least about 3:1, of at least about 5:1, of at least about 10:1, of at least about 20:1, of at least about 50:1, or of at least about 75:1.

In a further aspect, the compound inhibits in vitro PLD2 response. In a yet further aspect, the compound has a PLD2 IC₅₀ of less than about 10 μM, of less than about 1 μM, of less than about 500 nM, of less than about 100 nM, of less than about 60 nM, or of less than about 20 nM. In a still further aspect, the compound exhibits a PLD2:PLD1 inhibition ratio of at least about 2:1, of at least about 3:1, of at least about 5:1, of at least about 10:1, of at least about 20:1, of at least about 50:1, or of at least about 75:1.

In a further aspect, the compound of the method inhibits HIV replication. In a still further aspect, the compound inhibits HIV replication in activated CD4⁺ T-lymphocytes. In yet a further aspect, the compound inhibits HIV replication in tissue macrophages. In an even further aspect, the tissue macrophage is a brain macrophage. In a still further aspect, the tissue macrophage is a microglial cell. In yet a further aspect, the compound inhibits HIV replication in monocytes, dendritic cells, and activated CD4⁺ T-lymphocytes. In an even further aspect, the compound inhibits HIV replication in monocytes. In a still further aspect, the compound inhibits HIV replication in dendritic cells. In a yet further aspect, the compound inhibits HIV replication in activated CD4⁺ T-lymphocytes.

In a further aspect, the compound of the method inhibits HIV integration.

In a further aspect, the HIV infection comprises an HIV-1 serotype virus. In a still further aspect, the HIV-1 infection comprises a Group M, Group N, Group O, or Group P virus strain. In yet a further aspect, the HIV-1 infection comprises a Group M virus strain. In an even further aspect, the HIV-1 Group M virus strain is selected from the subtypes A, B, C, D, F, G, H, J, and K. In a still further aspect, the HIV-1 Group M virus strain subtype is subtype A. In yet a further aspect, the HIV-1 Group M virus strain subtype is subtype B. In an even further aspect, the HIV-1 Group M virus strain subtype is subtype C. In a still further aspect, the HIV-1 Group M virus strain subtype is subtype D. In yet a further aspect, the HIV-1 Group M virus strain subtype is subtype H. In an even further aspect, the HIV-1 Group M virus strain subtype comprises a circulating recombinant form (“CRF”) comprising genetic material from one or more subtypes selected from subtypes A, B, C, D, F, G, H, J, and K. In a still further aspect, the circulating recombinant form is CRF A/E. In yet a further aspect, the circulating recombinant form is CRF A/G.

In a further aspect, the HIV infection of the method comprises an HIV-2 serotype virus.

In a further aspect, the HIV infection of the method is associated with a disease selected from AIDS, aspergillosis, atypical mycobacteriosis, bacillary angiomatosis, bacteremia, bacterial pneumonia, bacterial sinusitis, candidiasis, CMV, CMV retinitis, coccidioidomycosis, cryptococcosis, cryptosporidiosis-isosporiasis, non-specific enteritis, folliculitis, herpes, histoplasmosis, HIV dementia, HIV meningitis, leismaniasis, Mycobacterium avium complex disease, nocardiosis, pencilliosis, progressive multifocal leukoencephalopathy (PML; or HIV encephalitis), Pneumocystis carinii pneumonia (PCP), pneumonia, Pseudomonas pneumonia, toxoplasma encephalitis, toxoplasmosis, tuberculosis, Kaposi sarcoma, lymphoma, and squamous cell carcinoma. In yet a further aspect, the lymphoma is selected from Non-Hodgkin's lymphoma, CNS lymphoma, primary lymphoma of the brain, and systemic lymphoma.

In a further aspect, the HIV infection of the method is associated with a cancer. In a still further aspect, the cancer is selected from a lymphoma, sarcoma, and a carcinoma. In yet a further aspect, the carcinoma is a squamous cell carcinoma. In an even further aspect, the sarcoma is Kaposi sarcoma. In a still further aspect, the lymphoma is selected from Non-Hodgkin's lymphoma, CNS lymphoma, primary lymphoma of the brain, and systemic lymphoma.

In a further aspect, the HIV infection of the method is associated with an opportunistic infection. In a still further aspect, the opportunistic infection is selected from aspergillosis, atypical mycobacteriosis, bacillary angiomatosis, bacteremia, bacterial pneumonia, bacterial sinusitis, candidiasis, CMV retinitis, coccidioidomycosis, cryptococcosis, cryptosporidiosis-isosporiasis, non-specific enteritis, folliculitis, herpes, histoplasmosis, HIV dementia, HIV meningitis, leismaniasis, Mycobacterium avium complex disease, nocardiosis, pencilliosis, progressive multifocal leukoencephalopathy (PML; or HIV encephalitis), Pneumocystis carinii pneumonia (PCP), pneumonia, Pseudomonas pneumonia, toxoplasma encephalitis, toxoplasmosis, and tuberculosis.

In a further aspect, the HIV infection of the method is associated with an infection associated with Cryptosporidium muris, Isospora belli, Toxoplasma gondii, Candida sp., Coccidioides immitis, Histoplasma capsulatum, Pneumocystis carnii, Mycobacterium avium complex, Mycobacterium tuberculosis, Cytomegalovirus, Epstein-Barr virus, Herpes simplex virus, Papovirus J-C, or Varicella-zoster.

In a further aspect, the HIV infection of the method comprises an HIV virus that is resistant to treatment with a non-nucleoside reverse transcriptase inhibitor. In a still further aspect, the HIV virus resistant to treatment with a non-nucleoside reverse transcriptase inhibitor has at least one mutation in the HIV reverse transcriptase. In yet a further aspect, the at least one mutation in the HIV reverse transcriptase is selected from 100I, 103N, 106A, 106M, 108I, 181C, 181I, 188C, 188H, 188L, 190A, 190S, 225H, 230L, and 236L. In an even further aspect, the at least one mutation is at amino acid position 100, 103, 106, 108, 181, 188, 190, 225, 230, or 236 of the HIV reverse transcriptase. In a still further aspect, the non-nucleoside reverse transcriptase inhibitor is selected from delavirdine, efavirenz, etravirine, nevirapine, rilpivirine, and lersivirine, or a pharmaceutically acceptable prodrug, salt, solvate, or polymorph thereof. In yet a further aspect, the non-nucleoside reverse transcriptase inhibitor is delavirdine, or a pharmaceutically acceptable prodrug, salt, solvate, or polymorph thereof. In an even further aspect, the non-nucleoside reverse transcriptase inhibitor is efavirenz, or a pharmaceutically acceptable prodrug, salt, solvate, or polymorph thereof. In a still further aspect, the non-nucleoside reverse transcriptase inhibitor is etravirine, or a pharmaceutically acceptable prodrug, salt, solvate, or polymorph thereof. In yet a further aspect, the non-nucleoside reverse transcriptase inhibitor is nevirapine, or a pharmaceutically acceptable prodrug, salt, solvate, or polymorph thereof. In an even further aspect, the non-nucleoside reverse transcriptase inhibitor is rilpivirine, or a pharmaceutically acceptable prodrug, salt, solvate, or polymorph thereof. In a still further aspect, the non-nucleoside reverse transcriptase inhibitor is lersivirine, or a pharmaceutically acceptable prodrug, salt, solvate, or polymorph thereof.

In a further aspect, the HIV infection of the method comprises an HIV virus that is resistant to treatment with a nucleoside reverse transcriptase inhibitor. In a still further aspect, the HIV virus resistant to treatment with a nucleoside reverse transcriptase inhibitor has at least one mutation in the HIV reverse transcriptase. In yet a further aspect, the at least one mutation in the HIV reverse transcriptase is selected from 41L, 44D, 62V, 65R, 67N, 69A, 69D, 69N, 69S, 69 insertion, 70R, 74V, 75I, 77L, 115F, 116Y, 118I, 151M, 184I, 184V, 210W, 215C, 215D, 215E, 215F, 215I, 215S, 215Y, 219E, and 219Q. In an even further aspect, the at least one mutation is at amino acid position 41, 44, 62, 65, 67, 69, 70, 74, 77, 115, 116, 118, 151, 184, 210, 215 or 219 of the HIV reverse transcriptase. In a still further aspect, the nucleoside reverse transcriptase inhibitor is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In yet a further aspect, the nucleoside reverse transcriptase inhibitor is selected from abacavir, didansine, emtricitabine, lamivudine, stavudine, tenofovir, zidovudine, and elvucitabine, or a pharmaceutically acceptable prodrug, salt, solvate, or polymorph thereof. In an even further aspect, the nucleoside reverse transcriptase inhibitor is abacavir, or a pharmaceutically acceptable prodrug, salt, solvate, or polymorph thereof. In a still further aspect, the nucleoside reverse transcriptase inhibitor is didansine, or a pharmaceutically acceptable prodrug, salt, solvate, or polymorph thereof. In yet a further aspect, the nucleoside reverse transcriptase inhibitor is emtricitabine, or a pharmaceutically acceptable prodrug, salt, solvate, or polymorph thereof. In an even further aspect, the nucleoside reverse transcriptase inhibitor is lamivudine, or a pharmaceutically acceptable prodrug, salt, solvate, or polymorph thereof. In a still further aspect, the nucleoside reverse transcriptase inhibitor is stavudine, or a pharmaceutically acceptable prodrug, salt, solvate, or polymorph thereof. In yet a further aspect, the nucleoside reverse transcriptase inhibitor is tenofovir, or a pharmaceutically acceptable prodrug, salt, solvate, or polymorph thereof. In an even further aspect, the nucleoside reverse transcriptase inhibitor is zidovudine, or a pharmaceutically acceptable prodrug, salt, solvate, or polymorph thereof. In a still further aspect, the nucleoside reverse transcriptase inhibitor is elvucitabine, or a pharmaceutically acceptable prodrug, salt, solvate, or polymorph thereof.

In a further aspect, the HIV infection of the method comprises an HIV virus that is resistant to treatment with a protease inhibitor. In a still further aspect, the HIV virus resistant to treatment with a protease inhibitor has at least one mutation in the HIV protease. In yet a further aspect, the at least one mutation in the HIV protease is selected from 30N, 46I, 46L, 48V, 50V, 82A, 82F, 82S, 82T, 84V, and 90M. In an even further aspect, the at least one mutation is at amino acid position 30, 46, 48, 50, 82, 84, or 90 of the HIV protease. In a still further aspect, the protease inhibitor is selected from atazanavir, darunavir, fosamprenavir, indinavir, lopinavir, nelfinavir, ritonavir, saquinavir, tipranavir, and lopinavir/ritonavir, or a pharmaceutically acceptable prodrug, salt, solvate, or polymorph thereof. In yet a further aspect, the protease inhibitor is atazanavir, or a pharmaceutically acceptable prodrug, salt, solvate, or polymorph thereof. In an even further aspect, the protease inhibitor is darunavir, or a pharmaceutically acceptable prodrug, salt, solvate, or polymorph thereof. In a still further aspect, the protease inhibitor is fosamprenavir, or a pharmaceutically acceptable prodrug, salt, solvate, or polymorph thereof. In yet a further aspect, the protease inhibitor is indinavir, or a pharmaceutically acceptable prodrug, salt, solvate, or polymorph thereof. In an even further aspect, the protease inhibitor is lopinavir, or a pharmaceutically acceptable prodrug, salt, solvate, or polymorph thereof. In a still further aspect, the protease inhibitor is nelfinavir, or a pharmaceutically acceptable prodrug, salt, solvate, or polymorph thereof. In yet a further aspect, the protease inhibitor is ritonavir, or a pharmaceutically acceptable prodrug, salt, solvate, or polymorph thereof. In an even further aspect, the protease inhibitor is saquinavir, or a pharmaceutically acceptable prodrug, salt, solvate, or polymorph thereof. In a still further aspect, the protease inhibitor is tipranavir, or a pharmaceutically acceptable prodrug, salt, solvate, or polymorph thereof. In yet a further aspect, the protease inhibitor is lopinavir/ritonavir, or a pharmaceutically acceptable prodrug, salt, solvate, or polymorph thereof.

In a further aspect, the HIV infection of the method comprises an HIV virus that is resistant to treatment with an integrase inhibitor. In a still further aspect, the integrase inhibitor is selected from raltegravir, dolutegravir, elvitegravir, and S/GSK1265744, or a pharmaceutically acceptable prodrug, salt, solvate, or polymorph thereof. In yet a further aspect, the integrase inhibitor is selected from raltegravir, dolutegravir, and elvitegravir, or a pharmaceutically acceptable prodrug, salt, solvate, or polymorph thereof. In an even further aspect, the integrase inhibitor is raltegravir, or a pharmaceutically acceptable prodrug, salt, solvate, or polymorph thereof. In a still further aspect, the integrase inhibitor is dolutegravir, or a pharmaceutically acceptable prodrug, salt, solvate, or polymorph thereof. In yet a further aspect, the integrase inhibitor is elvitegravir, or a pharmaceutically acceptable prodrug, salt, solvate, or polymorph thereof.

In a further aspect, the HIV infection of the method comprises an HIV virus that is resistant to treatment with a fusion inhibitor. In a still further aspect, the fusion inhibitor is selected from enfuvirtide, maraviroc, cenicriviroc, ibalizumab, BMS-663068, and PRO-140, or a pharmaceutically acceptable prodrug, salt, solvate, or polymorph thereof. In yet a further aspect, the fusion inhibitor is selected from enfuvirtide, maraviroc, cenicriviroc, and ibalizumab, or a pharmaceutically acceptable prodrug, salt, solvate, or polymorph thereof. In an even further aspect, the fusion inhibitor is enfuvirtide, or a pharmaceutically acceptable prodrug, salt, solvate, or polymorph thereof. In a still further aspect, the fusion inhibitor is maraviroc, or a pharmaceutically acceptable prodrug, salt, solvate, or polymorph thereof. In yet a further aspect, the fusion inhibitor is cenicriviroc, or a pharmaceutically acceptable prodrug, salt, solvate, or polymorph thereof. In an even further aspect, the fusion inhibitor is ibalizumab, or a pharmaceutically acceptable prodrug, salt, solvate, or polymorph thereof.

In a further aspect, the method further comprises assessing viral load in the subject following administration.

In a further aspect, the method further comprises administering to the subject a non-PLD anti-HIV therapy.

In a further aspect, the administering of the method comprises inhalation or oral administration. In a still further aspect, the administering of the method comprises intravenous or intra-arterial injection.

5. Treating an HIV Infection by Administering a PLD Inhibitor

In various aspects, the invention relates to a method for treating a subject for HIV infection, the method comprising the step of administering to the subject an effective amount of a phospholipase D (PLD) inhibitor, thereby treating the subject for HIV infection.

In a further aspect, the subject of the method is mammalian. In a still further aspect, the subject of the method is human. In a yet further aspect, the subject has been diagnosed with a need of treatment for HIV infection prior to the administering step. In an even further aspect, the method further comprises the step of identifying the subject as having a need of treatment for HIV infection.

In a further aspect, the amount of the method is a therapeutically effective amount. In a yet further aspect, the amount of the method is a prophylactically effective amount.

In a further aspect, the method further comprises assessing viral load in the subject following administration.

In a further aspect, the PLD inhibited is PLD1. In a still further aspect, the phospholipase D (PLD) inhibitor is a PLD1-selective inhibitor. In an even further aspect, the the phospholipase D (PLD) inhibitor inhibits PLD1 response in an in vitro assay comprising a cultured cell-line. In a yet further aspect, the phospholipase D (PLD) inhibitor inhibits PLD1 response in Calu-1 cells.

In a further aspect, the PLD inhibited is PLD2. In a still further aspect, the phospholipase D (PLD) inhibitor is a PLD2-selective inhibitor. In a yet further aspect, the phospholipase D (PLD) inhibitor inhibits PLD2 response in HEK293gfpPLD2 cells.

In a further aspect, the phospholipase D (PLD) inhibitor inhibits in vitro PLD1 response. In a still further aspect, the phospholipase D (PLD) inhibitor has a PLD1 IC₅₀ of less than about 10 μM, of less than about 1 μM, of less than about 500 nM, of less than about 100 nM, of less than about 60 nM, or of less than about 20 nM. In a yet further aspect, the phospholipase D (PLD) inhibitor exhibits a PLD1:PLD2 inhibition ratio of at least about 2:1, of at least about 3:1, of at least about 5:1, of at least about 10:1, of at least about 20:1, of at least about 50:1, or of at least about 75:1.

In a further aspect, the phospholipase D (PLD) inhibitor inhibits in vitro PLD2 response. In a still further aspect, the phospholipase D (PLD) inhibitor has a PLD2 IC₅₀ of less than about 10 μM, of less than about 1 μM, of less than about 500 nM, of less than about 100 nM, of less than about 60 nM, or of less than about 20 nM. In a yet further aspect, the phospholipase D (PLD) inhibitor exhibits a PLD2:PLD1 inhibition ratio of at least about 2:1, of at least about 3:1, of at least about 5:1, of at least about 10:1, of at least about 20:1, of at least about 50:1, or of at least about 75:1.

In a further aspect, the phospholipase D (PLD) inhibitor of the method for treating a subject for HIV infection is a compound having a structure represented by a formula:

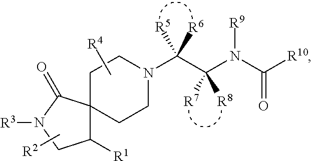

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, trifluoromethyl, carboxamido, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the phospholipase D (PLD) inhibitor of the method for treating a subject for HIV infection is a compound having a structure represented by a formula:

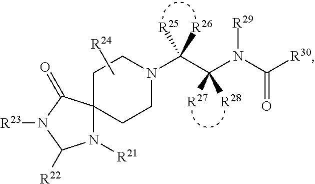

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, trifluoromethyl, carboxamido, alkylsulfonyl, an optionally substituted C1 to C6 alkyl, or an optionally substituted C3 to C6 cycloalkyl or R²⁵ and R²⁶, together with the intermediate carbon, comprise an optionally substituted C3 to C6 cycloalkyl; wherein each of R²⁷ and R²⁸ independently comprises hydrogen, trifluoromethyl, carboxamido, alkylsulfonyl, an optionally substituted C1 to C6 alkyl, or an optionally substituted C3 to C6 cycloalkyl or R²⁷ and R²⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the phospholipase D (PLD) inhibitor of the method for treating a subject for HIV infection is a compound having a structure represented by a formula:

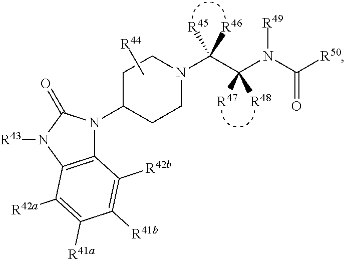

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, trifluoromethyl, carboxamido, alkylsulfonyl, an optionally substituted C1 to C6 alkyl, or an optionally substituted C3 to C6 cycloalkyl or R⁴⁵ and R⁴⁶, together with the intermediate carbon, comprise an optionally substituted C3 to C6 cycloalkyl; wherein each of R⁴⁷ and R⁴⁸ independently comprises hydrogen, trifluoromethyl, carboxamido, alkylsulfonyl, an optionally substituted C1 to C6 alkyl, or an optionally substituted C3 to C6 cycloalkyl or R⁴⁷ and R⁴⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the phospholipase D (PLD) inhibitor of the method for treating a subject for HIV infection is a compound selected from: trans-diethylstilbestrol, resveratrol, honokiol, SCH420789, presqualene diphosphate, raloxifene, 4-hydroxy tamoxifen, 5-fluoro-2-indoyl des-chlorohalopemide, and halopemide, or a pharmaceutically acceptable salt, hydrate, solvate, or polymorph thereof, thereby treating the subject for HIV infection.

In a further aspect, the phospholipase D (PLD) inhibitor of the method for treating a subject for HIV infection is a compound selected from:

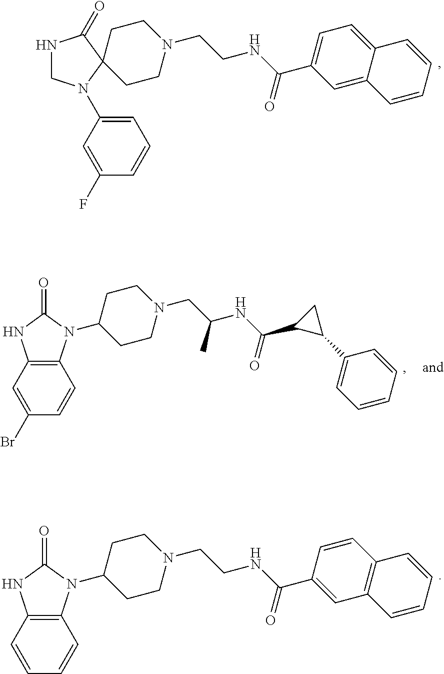

In a further aspect, the method further comprises administering to the subject a non-PLD anti-HIV therapy.

In a further aspect, the administering of the method comprises inhalation or oral administration. In a still further aspect, the administering of the method comprises intravenous or intra-arterial injection.

6. Treating an HIV Infection by Administering a Compound that Binds PLD in a Non-Catalytic Domain

In one aspect, the invention relates to a method for treating a subject for HIV infection, the method comprising the step of administering to the subject an effective amount of a binding agent of phospholipase D (PLD), wherein the binding agent binds to at least one amino acid in a non-catalytic domain of PLD, thereby inhibiting viral replication within the cell.

In a further aspect, the binding to PLD of the method is allosteric binding.

In a further aspect, the non-catalytic domain of the method comprises at least one amino acid residue in amino acids 1-505 of PLD1, or the homologous amino acids of PLD2. In a yet further aspect, the non-catalytic domain comprises at least one amino acid in amino acids 81-425 of PLD1, or the homologous amino acids of PLD2. In a still further aspect, the non-catalytic domain comprises at least one amino acid in amino acids 200-390 of PLD1, or the homologous amino acids of PLD2. In an even further aspect, the non-catalytic domain comprises at least one amino acid in amino acids 310-375, or the homologous amino acids of PLD2. In a still further aspect, the binding agent binds a domain comprising amino acids 310-375.

In a further aspect, the subject of the method has been diagnosed with a need of treatment for HIV infection prior to the administering step. In a still further aspect, the method further comprises the step of identifying the subject as having a need of treatment for HIV infection.

In a further aspect, the amount administered in the method is a therapeutically effective amount. In a still further aspect, the amount administered in the method is a prophylactically effective amount.

In a further aspect, the method further comprises assessing viral load in the subject following administration.

In a further aspect, the method further comprises administering to the subject a non-PLD anti-HIV therapy.

In a further aspect, the administering of the method comprises inhalation or oral administration. In a still further aspect, the administering of the method comprises intravenous or intra-arterial injection.

In one aspect, the invention relates to a method for treating a subject for HIV infection, the method comprising the step of administering to the subject an effective amount of an allosteric binding agent of phospholipase D (PLD), thereby treating the subject for HIV infection.

In a further aspect, the allosteric binding of the method occurs with at least one amino acid residue in amino acids 1-505 of PLD1, or the homologous amino acids of PLD2, thereby inhibiting viral entry into the cell. In a still further aspect, the allosteric binding of the method occurs with at least one amino acid residue in amino acids 81-425 of PLD1, or the homologous amino acids of PLD2. In a yet further aspect, the allosteric binding of the method occurs with at least one amino acid residue in amino acids 200-390 of claim 84, wherein PLD1, or the homologous amino acids of PLD2. In an even further aspect, the allosteric binding of the method occurs with at least one amino acid residue in amino acids 310-375, or the homologous amino acids of PLD2. In a still further aspect, the allosteric binding of the method occurs with a domain comprising amino acids 310-375.

In a further aspect, the subject of the method has been diagnosed with a need of treatment for HIV infection prior to the administering step. In a still further aspect, the method further comprises the step of identifying the subject as having a need of treatment for HIV infection.

In a further aspect, the amount administered in the method is a therapeutically effective amount. In a still further aspect, the amount administered in the method is a prophylactically effective amount.

In a further aspect, the method further comprises assessing viral load in the subject following administration.

In a further aspect, the method further comprises administering to the subject a non-PLD anti-HIV therapy.

In a further aspect, the administering of the method comprises inhalation or oral administration. In a still further aspect, the administering of the method comprises intravenous or intra-arterial injection.

In a further aspect, the agent of the foregoing methods for treating a subject for HIV infection is a compound having a structure represented by a formula:

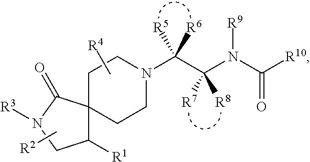

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, trifluoromethyl, carboxamido, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the agent of the foregoing methods for treating a subject for HIV infection is a compound having a structure represented by a formula:

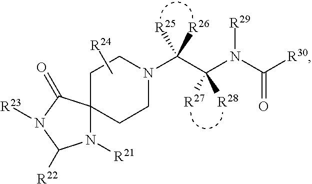

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, trifluoromethyl, carboxamido, alkylsulfonyl, an optionally substituted C1 to C6 alkyl, or an optionally substituted C3 to C6 cycloalkyl or R²⁵ and R²⁶, together with the intermediate carbon, comprise an optionally substituted C3 to C6 cycloalkyl; wherein each of R²⁷ and R²⁸ independently comprises hydrogen, trifluoromethyl, carboxamido, alkylsulfonyl, an optionally substituted C1 to C6 alkyl, or an optionally substituted C3 to C6 cycloalkyl or R²⁷ and R²⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the agent of the foregoing methods for treating a subject for HIV infection is a compound having a structure represented by a formula:

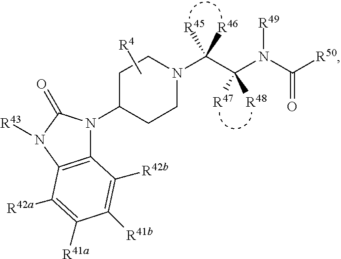

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, trifluoromethyl, carboxamido, alkoxy, thiol, alkylsulfonyl, an optionally substituted C1 to C6 alkyl, or an optionally substituted C3 to C6 cycloalkyl or R⁴⁵ and R⁴⁶, together with the intermediate carbon, comprise an optionally substituted C3 to C6 cycloalkyl; wherein each of R⁴⁷ and R⁴⁸ independently comprises hydrogen, trifluoromethyl, carboxamido, alkylsulfonyl, an optionally substituted C1 to C6 alkyl, or an optionally substituted C3 to C6 cycloalkyl or R⁴⁷ and R⁴⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the agent of the foregoing methods for treating a subject for HIV infection is a compound selected from: trans-diethylstilbestrol, resveratrol, honokiol, SCH420789, presqualene diphosphate, raloxifene, 4-hydroxy tamoxifen, 5-fluoro-2-indoyl des-chlorohalopemide, and halopemide, or a pharmaceutically acceptable salt, hydrate, solvate, or polymorph thereof, thereby treating the subject for HIV infection.

7. Treating an HIV Infection by Administering a Compound that Binds PLD in a Catalytic Domain

In one aspect, the invention relates to a method for treating a subject for HIV infection, the method comprising the step of administering to the subject an effective amount of a binding agent of phospholipase D (PLD), wherein the binding agent binds to at least one amino acid in a non-catalytic domain of PLD, thereby inhibiting viral replication within the cell.

In a further aspect, the binding to PLD of the method is allosteric binding. In a still further aspect, the binding to PLD of the method is orthosteric binding.

In a further aspect, the non-catalytic domain of the method comprises at least one amino acid residue in amino acids 463-928 of PLD1, or the homologous amino acids of PLD2.

In a further aspect, the subject of the method has been diagnosed with a need of treatment for HIV infection prior to the administering step. In a still further aspect, the method further comprises the step of identifying the subject as having a need of treatment for HIV infection.

In a further aspect, the amount administered in the method is a therapeutically effective amount. In a still further aspect, the amount administered in the method is a prophylactically effective amount.

In a further aspect, the method further comprises assessing viral load in the subject following administration.

In a further aspect, the method further comprises administering to the subject a non-PLD anti-HIV therapy.

In a further aspect, the administering of the method comprises inhalation or oral administration. In a still further aspect, the administering of the method comprises intravenous or intra-arterial injection.

In one aspect, the invention relates to a method for treating a subject for HIV infection, the method comprising the step of administering to the subject an effective amount of an allosteric binding agent of phospholipase D (PLD), thereby treating the subject for HIV infection. In a further aspect, the invention relates to a method for treating a subject for HIV infection, the method comprising the step of administering to the subject an effective amount of an orthosteric binding agent of phospholipase D (PLD), thereby treating the subject for HIV infection.

In a further aspect, the allosteric binding of the method occurs with at least one amino acid residue in amino acids 463-928 of PLD1, or the homologous amino acids of PLD2, thereby inhibiting viral entry into the cell.

In a further aspect, the subject of the method has been diagnosed with a need of treatment for HIV infection prior to the administering step. In a still further aspect, the method further comprises the step of identifying the subject as having a need of treatment for HIV infection.

In a further aspect, the amount administered in the method is a therapeutically effective amount. In a still further aspect, the amount administered in the method is a prophylactically effective amount.

In a further aspect, the method further comprises assessing viral load in the subject following administration.

In a further aspect, the method further comprises administering to the subject a non-PLD anti-HIV therapy.

In a further aspect, the administering of the method comprises inhalation or oral administration. In a still further aspect, the administering of the method comprises intravenous or intra-arterial injection.

8. Treating an HIV Infection by Co-Administering Two or More Therapeutic Agents

In one aspect, the invention relates to a method for treating a subject comprising the step of co-administering an effective amount of to or more therapeutic agents to the subject; wherein the subject has been diagnosed with a need for treatment of an HIV infection prior to the administering step; and wherein the combination of two or more therapeutic agents comprises: a) a phospholipase D inhibitor; and b) one or more therapeutic agents selected from: i) an HIV fusion/lysis inhibitor, or a pharmaceutically acceptable prodrug, salt, solvate, or polymorph thereof; ii) an HIV integrase inhibitor, or a pharmaceutically acceptable prodrug, salt, solvate, or polymorph thereof; iii) an HIV non-nucleoside reverse transcriptase inhibitor, or a pharmaceutically acceptable prodrug, salt, solvate, or polymorph thereof; iv) an HIV nucleoside reverse transcriptase inhibitor, or a pharmaceutically acceptable prodrug, salt, solvate, or polymorph thereof; and v) an HIV protease inhibitor, or a pharmaceutically acceptable prodrug, salt, solvate.

In a further aspect, the HIV infection of the method comprises an HIV-1 serotype virus. In a still further aspect, the HIV-1 infection comprises a Group M, Group N, Group O, or Group P virus strain. In yet a further aspect, the HIV-1 infection is a Group M virus strain. In an even further aspect, the HIV-1 Group M virus strain is selected from the subtypes A, B, C, D, F, G, H, J, and K. In a still further aspect, the HIV-1 Group M virus strain subtype is subtype A. In yet a further aspect, the HIV-1 Group M virus strain subtype is subtype B. In an even further aspect, the HIV-1 Group M virus strain subtype is subtype C. In a still further aspect, the HIV-1 Group M virus strain subtype is subtype D. In yet a further aspect, the HIV-1 Group M virus strain subtype is subtype H. In an even further aspect, the HIV-1 Group M virus strain subtype is a circulating recombinant form (“CRF”) comprising genetic material from one or more subtypes selected from subtypes A, B, C, D, F, G, H, J, and K. In a still further aspect, the circulating recombinant form is CRF A/E. In yet a further aspect, the circulating recombinant form is CRF A/G.

In a further aspect, the HIV infection of the method comprises an HIV-2 serotype virus.

In a further aspect, the HIV infection of the method is associated with a disease selected from AIDS, aspergillosis, atypical mycobacteriosis, bacillary angiomatosis, bacteremia, bacterial pneumonia, bacterial sinusitis, candidiasis, CMV, CMV retinitis, coccidioidomycosis, cryptococcosis, cryptosporidiosis-isosporiasis, non-specific enteritis, folliculitis, herpes, histoplasmosis, HIV dementia, HIV meningitis, leismaniasis, Mycobacterium avium complex disease, nocardiosis, pencilliosis, progressive multifocal leukoencephalopathy (PML; or HIV encephalitis), Pneumocystis carinii pneumonia (PCP), pneumonia, Pseudomonas pneumonia, toxoplasma encephalitis, toxoplasmosis, tuberculosis, Kaposi sarcoma, lymphoma, and squamous cell carcinoma.

In a further aspect, the effective amount is a therapeutically effective amount. In a still the effective amount is a prophylactically effective amount.

In a further aspect, the effective amount of a phospholipase D inhibitor inhibits HIV replication. In a still further aspect, the effective amount of a phospholipase D inhibitor inhibits HIV integration.

In a further aspect, the subject is a mammal. In a still further aspect, the subject is a human.

In a further aspect, co-administration is administration in a substantially simultaneous manner. In a still further aspect, simultaneous administration comprises a single dose form containing a fixed ratio of the phospholipase D inhibitor and the one or more therapeutic agents. In yet a further aspect, the single dose form is a capsule or a tablet. In an even further aspect, the single dose form is an ampule for a single intravenous administration. In a still further aspect, simultaneous administration comprises a single dose forms for each of the phospholipase D inhibitor and the one or more therapeutic agents. In yet a further aspect, the single dose form is a capsule or a tablet. In an even further aspect, the single dose form is an ampule for a single intravenous administration.

In a further aspect, co-administration is administration in a substantially sequential manner.

In a further aspect, the phospholipase D inhibitor is a disclosed phospholipase D inhibitor. In a still further aspect, the phospholipase D inhibitor inhibits PLD1 and/or PLD2. In yet a further aspect, the phospholipase D inhibitor inhibits PLD1. In an even further aspect, the phospholipase D inhibitor inhibits PLD2.

In a further aspect, the phospholipase D inhibitor is selected from:

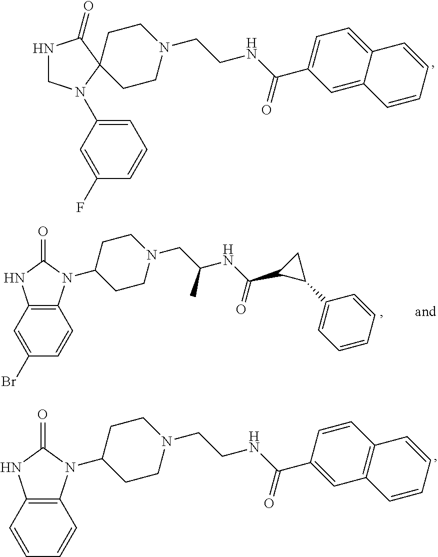

or a pharmaceutically acceptable salt, hydrate, solvate, or polymorph thereof.

In a further aspect, the HIV fusion/lysis inhibitor of the method is selected from enfuvirtide, maraviroc, cenicriviroc, ibalizumab, BMS-663068, and PRO-140, or a pharmaceutically acceptable prodrug, salt, solvate, or polymorph thereof. In a still further aspect, the HIV fusion/lysis inhibitor is selected from enfuvirtide, maraviroc, cenicriviroc, and ibalizumab, or a pharmaceutically acceptable prodrug, salt, solvate, or polymorph thereof. In yet a further aspect, the HIV fusion/lysis inhibitor is enfuvirtide, or a pharmaceutically acceptable prodrug, salt, solvate, or polymorph thereof. In an even further aspect, the HIV fusion/lysis inhibitor is maraviroc, or a pharmaceutically acceptable prodrug, salt, solvate, or polymorph thereof. In a still further aspect, the HIV fusion/lysis inhibitor is cenicriviroc, or a pharmaceutically acceptable prodrug, salt, solvate, or polymorph thereof. In yet a further aspect, the HIV fusion/lysis inhibitor is ibalizumab, or a pharmaceutically acceptable prodrug, salt, solvate, or polymorph thereof.

In a further aspect, the HIV integrase inhibitor of the method is selected from raltegravir, dolutegravir, elvitegravir, and S/GSK1265744, or a pharmaceutically acceptable prodrug, salt, solvate, or polymorph thereof. In a still further aspect, the HIV integrase inhibitor is selected from raltegravir, dolutegravir, and elvitegravir, or a pharmaceutically acceptable prodrug, salt, solvate, or polymorph thereof. In yet a further aspect, the HIV integrase inhibitor is raltegravir, or a pharmaceutically acceptable prodrug, salt, solvate, or polymorph thereof. In an even further aspect, the HIV integrase inhibitor is dolutegravir, or a pharmaceutically acceptable prodrug, salt, solvate, or polymorph thereof. In a still further aspect, the HIV integrase inhibitor is elvitegravir, or a pharmaceutically acceptable prodrug, salt, solvate, or polymorph thereof.

In a further aspect, the non-nucleoside reverse transcriptase inhibitor of the method is selected from delavirdine, efavirenz, etravirine, nevirapine, rilpivirine, and lersivirine, or a pharmaceutically acceptable prodrug, salt, solvate, or polymorph thereof. In a still further aspect, the non-nucleoside reverse transcriptase inhibitor is delavirdine, or a pharmaceutically acceptable prodrug, salt, solvate, or polymorph thereof. In yet a further aspect, the non-nucleoside reverse transcriptase inhibitor is efavirenz, or a pharmaceutically acceptable prodrug, salt, solvate, or polymorph thereof. In an even further aspect, the non-nucleoside reverse transcriptase inhibitor is etravirine, or a pharmaceutically acceptable prodrug, salt, solvate, or polymorph thereof. In a still further aspect, the non-nucleoside reverse transcriptase inhibitor is nevirapine, or a pharmaceutically acceptable prodrug, salt, solvate, or polymorph thereof. In yet a further aspect, the non-nucleoside reverse transcriptase inhibitor is rilpivirine, or a pharmaceutically acceptable prodrug, salt, solvate, or polymorph thereof. In an even further aspect, the non-nucleoside reverse transcriptase inhibitor is lersivirine, or a pharmaceutically acceptable prodrug, salt, solvate, or polymorph thereof.

In a further aspect, the nucleoside reverse transcriptase inhibitor of the method is selected from abacavir, didansine, emtricitabine, lamivudine, stavudine, tenofovir, zidovudine, elvucitabine, and GS-7340, or a pharmaceutically acceptable prodrug, salt, solvate, or polymorph thereof. In a still further aspect, the nucleoside reverse transcriptase inhibitor is selected from abacavir, didansine, emtricitabine, lamivudine, stavudine, tenofovir, zidovudine, and elvucitabine, or a pharmaceutically acceptable prodrug, salt, solvate, or polymorph thereof. In yet a further aspect, the nucleoside reverse transcriptase inhibitor is abacavir, or a pharmaceutically acceptable prodrug, salt, solvate, or polymorph thereof. In an even further aspect, the nucleoside reverse transcriptase inhibitor is didansine, or a pharmaceutically acceptable prodrug, salt, solvate, or polymorph thereof. In a still further aspect, the nucleoside reverse transcriptase inhibitor is elvucitabine, or a pharmaceutically acceptable prodrug, salt, solvate, or polymorph thereof. In yet a further aspect, the nucleoside reverse transcriptase inhibitor is emtricitabine, or a pharmaceutically acceptable prodrug, salt, solvate, or polymorph thereof. In an even further aspect, the nucleoside reverse transcriptase inhibitor is lamivudine, or a pharmaceutically acceptable prodrug, salt, solvate, or polymorph thereof. In a still further aspect, the nucleoside reverse transcriptase inhibitor is stavudine, or a pharmaceutically acceptable prodrug, salt, solvate, or polymorph thereof. In yet a further aspect, the nucleoside reverse transcriptase inhibitor is tenofovir, or a pharmaceutically acceptable prodrug, salt, solvate, or polymorph thereof. In an even further aspect, the nucleoside reverse transcriptase inhibitor is zidovudine, or a pharmaceutically acceptable prodrug, salt, solvate, or polymorph thereof.

In a further aspect, the protease inhibitor of the method is selected from atazanavir, darunavir, fosamprenavir, indinavir, nelfinavir, ritonavir, saquinavir, tipranavir, and lopinavir/ritonavir, or a pharmaceutically acceptable prodrug, salt, solvate, or polymorph thereof. In a still further aspect, the protease inhibitor is atazanavir, or a pharmaceutically acceptable prodrug, salt, solvate, or polymorph thereof. In yet a further aspect, the protease inhibitor is darunavir, or a pharmaceutically acceptable prodrug, salt, solvate, or polymorph thereof. In an even further aspect, the protease inhibitor is fosamprenavir, or a pharmaceutically acceptable prodrug, salt, solvate, or polymorph thereof. In a still further aspect, the protease inhibitor is indinavir, or a pharmaceutically acceptable prodrug, salt, solvate, or polymorph thereof. In yet a further aspect, the protease inhibitor is lopinaviror a pharmaceutically acceptable prodrug, salt, solvate, or polymorph thereof. In an even further aspect, the protease inhibitor is nelfinavir, or a pharmaceutically acceptable prodrug, salt, solvate, or polymorph thereof. In a still further aspect, the protease inhibitor is ritonavir, or a pharmaceutically acceptable prodrug, salt, solvate, or polymorph thereof. In yet a further aspect, the protease inhibitor is saquinavir, or a pharmaceutically acceptable prodrug, salt, solvate, or polymorph thereof. In an even further aspect, the protease inhibitor is tipranavir, or a pharmaceutically acceptable prodrug, salt, solvate, or polymorph thereof. In a still further aspect, the protease inhibitor is lopinavir/ritonavir, or a pharmaceutically acceptable prodrug, salt, solvate, or polymorph thereof.

### H. METHODS OF INHIBITING HIV REPLICATION IN CELLS

In one aspect, the method of use is directed to inhibition of HIV replication in cells. In a further aspect, the disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein which are usually applied in the treatment of the above mentioned pathological conditions.

1. Inhibiting HIV Replication in a Cell by Contacting the Cell with a 1-oxo-2,8-diazaspiro[4.5]decanyl analog

In one aspect, the invention relates to a method for inhibiting HIV replication in at least one cell, comprising the step of contacting the at least one cell with an effective amount of at least one compound having a structure represented by a formula:

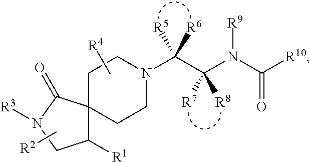

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting HIV replication in at least one cell.

In a further aspect, the compound of the method has a structure represented by a formula:

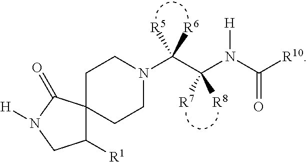

In a further aspect, the cell of the method is mammalian. In a still further aspect, the cell of the method is human. In a yet further aspect, the cell of the method has been isolated from a mammal prior to the contacting step.

In a further aspect, the at least one cell is an activated CD4⁺ T-lymphocyte. In a still further aspect, the at least one cell is a resting or memory T-cell. In yet a further aspect, the at least one cell is a tissue macrophage. In an even further aspect, the tissue macrophage is a brain macrophage. In a still further aspect, the tissue macrophage is a microglial cell.

In a further aspect, contacting is via administration to a subject. In a still further aspect, the subject has been diagnosed with a need for inhibiting HIV replication prior to the administering step. In yet a further aspect, the subject has been diagnosed with a need for treatment of HIV related to HIV replication prior to the administering step.

2. Inhibiting HIV Replication in a Cell by Contacting the Cell with a 4-oxo-1,3,8-triazaspiro[4.5]decanyl analog

In one aspect, the invention relates to a method for inhibiting HIV replication in at least one cell, comprising the step of contacting the at least one cell with an effective amount of at least one compound having a structure represented by a formula:

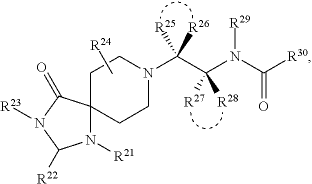

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting HIV replication in at least one cell.

In a further aspect, the compound of the method has a structure represented by a formula:

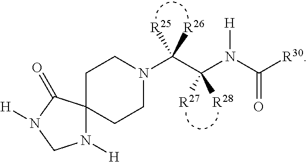

In a further aspect, the compound of the method is:

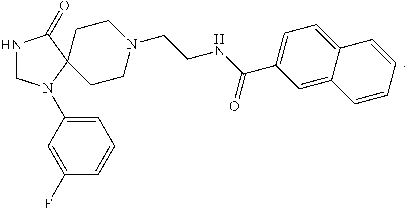 [SMILES: O=C(NCCN1CCC2(CC1)C(=O)NCN2c1cccc(F)c1)c1ccc2ccccc2c1]

In a further aspect, the cell of the method is mammalian. In a still further aspect, the cell of the method is human. In a yet further aspect, the cell of the method has been isolated from a mammal prior to the contacting step.

In a further aspect, the at least one cell is an activated CD4⁺ T-lymphocyte. In a still further aspect, the at least one cell is a resting or memory T-cell. In yet a further aspect, the at least one cell is a tissue macrophage. In an even further aspect, the tissue macrophage is a brain macrophage. In a still further aspect, the tissue macrophage is a microglial cell.

In a further aspect, contacting is via administration to a subject. In a still further aspect, the subject has been diagnosed with a need for inhibiting HIV replication prior to the administering step. In yet a further aspect, the subject has been diagnosed with a need for treatment of HIV related to HIV replication prior to the administering step.

3. Inhibiting HIV Replication in a Cell by Contacting the Cell with a Substituted 2-oxo-2,3-dihydro-1H-benzo[d]imidazol-1-yl analog

In one aspect, the invention relates to a method for inhibiting HIV replication in at least one cell, comprising the step of contacting the at least one cell with an effective amount of at least one compound having a structure represented by a formula:

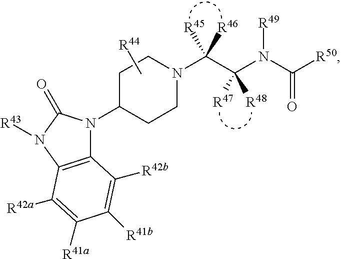

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting HIV replication in at least one cell.

In a further aspect, the compound of the method has a structure represented by a formula:

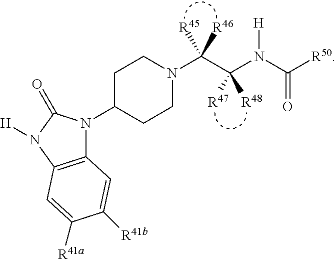

In a further aspect, the compound of the method is:

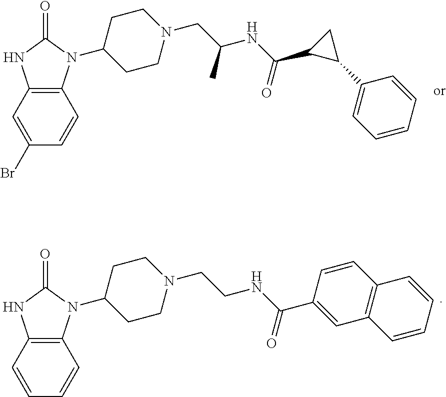

In a further aspect, the cell of the method is mammalian. In a still further aspect, the cell of the method is human. In a yet further aspect, the cell of the method has been isolated from a mammal prior to the contacting step.

In a further aspect, the at least one cell is an activated CD4⁺ T-lymphocyte. In a still further aspect, the at least one cell is a resting or memory T-cell. In yet a further aspect, the at least one cell is a tissue macrophage. In an even further aspect, the tissue macrophage is a brain macrophage. In a still further aspect, the tissue macrophage is a microglial cell.

In a further aspect, contacting is via administration to a subject. In a still further aspect, the subject has been diagnosed with a need for inhibiting HIV replication prior to the administering step. In yet a further aspect, the subject has been diagnosed with a need for treatment of HIV related to HIV replication prior to the administering step.

4. Inhibiting HIV Replication in a Cell by Contacting the Cell with a Selected Compound

In one aspect, the invention relates to a method for inhibiting HIV replication in at least one cell, comprising the step of contacting the at least one cell with an effective amount of at least one compound having a structure selected from: trans-diethylstilbestrol, resveratrol, honokiol, SCH420789, presqualene diphosphate, raloxifene, 4-hydroxytamoxifen, 5-fluoro-2-indoyl des-chlorohalopemide, and halopemide, or a pharmaceutically acceptable salt, hydrate, solvate, or polymorph thereof, thereby inhibiting HIV replication in at least one cell.

In one aspect, the invention relates to a method for inhibiting HIV replication in at least one cell, comprising the step of contacting the at least one cell with an effective amount of at least one compound having a structure represented by a formula:

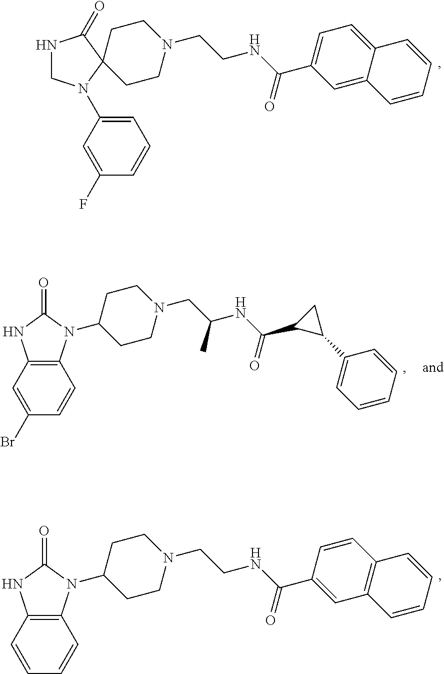

or a pharmaceutically acceptable salt, hydrate, solvate, or polymorph thereof, thereby inhibiting HIV replication in at least one cell.

In a further aspect, the cell of the method is mammalian. In a still further aspect, the cell of the method is human. In a yet further aspect, the cell of the method has been isolated from a mammal prior to the contacting step.

In a further aspect, the at least one cell is an activated CD4⁺ T-lymphocyte. In a still further aspect, the at least one cell is a resting or memory T-cell. In yet a further aspect, the at least one cell is a tissue macrophage. In an even further aspect, the tissue macrophage is a brain macrophage. In a still further aspect, the tissue macrophage is a microglial cell.

In a further aspect, contacting is via administration to a subject. In a still further aspect, the subject has been diagnosed with a need for inhibiting HIV replication prior to the administering step. In yet a further aspect, the subject has been diagnosed with a need for treatment of HIV related to HIV replication prior to the administering step.

5. Inhibiting HIV Replication in a Cell by Contacting the Cell with a PLD Inhibitor

In one aspect, the invention relates to a method for inhibiting HIV replication within a cell, the method comprising the step of contacting the cell with an effective amount of a phospholipase D (PLD) inhibitor, thereby inhibiting viral replication within the cell.

In a further aspect, the cell of the method is mammalian. In a still further aspect, the cell of the method is human. In a yet further aspect, the cell of the method has been isolated from a mammal prior to the contacting step.

In a further aspect, the PLD inhibited is PLD1. In a still further aspect, the phospholipase D (PLD) inhibitor is a PLD1-selective inhibitor. In an even further aspect, the phospholipase D (PLD) inhibitor inhibits PLD1 response in an in vitro assay comprising a cultured cell-line. In a yet further aspect, the phospholipase D (PLD) inhibitor inhibits PLD1 response in Calu-1 cells.

In a further aspect, the PLD inhibited is PLD2. In a still further aspect, the phospholipase D (PLD) inhibitor is a PLD2-selective inhibitor. In a yet further aspect, the phospholipase D (PLD) inhibitor inhibits PLD2 response in HEK293gfpPLD2 cells.

In a further aspect, the phospholipase D (PLD) inhibitor inhibits in vitro PLD1 response. In a still further aspect, the phospholipase D (PLD) inhibitor has a PLD1 IC₅₀ of less than about 10 μM, of less than about 1 μM, of less than about 500 nM, of less than about 100 nM, of less than about 60 nM, or of less than about 20 nM. In a yet further aspect, the phospholipase D (PLD) inhibitor exhibits a PLD1:PLD2 inhibition ratio of at least about 2:1, of at least about 3:1, of at least about 5:1, of at least about 10:1, of at least about 20:1, of at least about 50:1, or of at least about 75:1.

In a further aspect, the phospholipase D (PLD) inhibitor inhibits in vitro PLD2 response. In a still further aspect, the phospholipase D (PLD) inhibitor has a PLD2 IC₅₀ of less than about 10 μM, of less than about 1 μM, of less than about 500 nM, of less than about 100 nM, of less than about 60 nM, or of less than about 20 nM. In a yet further aspect, the phospholipase D (PLD) inhibitor exhibits a PLD2:PLD1 inhibition ratio of at least about 2:1, of at least about 3:1, of at least about 5:1, of at least about 10:1, of at least about 20:1, of at least about 50:1, or of at least about 75:1.

In a further aspect, the phospholipase D (PLD) inhibitor of the method for inhibiting HIV replication within a cell is a compound having a structure represented by a formula:

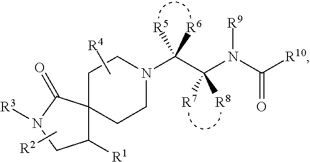

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, trifluoromethyl, carboxamido, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting viral replication within the cell.

In a further aspect, the phospholipase D (PLD) inhibitor of the method for inhibiting HIV replication within a cell is a compound having a structure represented by a formula:

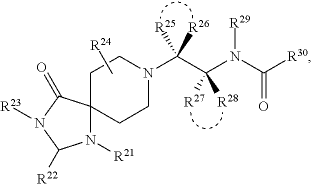

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, trifluoromethyl, carboxamido, alkylsulfonyl, an optionally substituted C1 to C6 alkyl, or an optionally substituted C3 to C6 cycloalkyl or R²⁵ and R²⁶, together with the intermediate carbon, comprise an optionally substituted C3 to C6 cycloalkyl; wherein each of R²⁷ and R²⁸ independently comprises hydrogen, trifluoromethyl, carboxamido, alkylsulfonyl, an optionally substituted C1 to C6 alkyl, or an optionally substituted C3 to C6 cycloalkyl or R²⁷ and R²⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting viral replication within the cell.

In a further aspect, the phospholipase D (PLD) inhibitor of the method for inhibiting HIV replication within a cell is a compound having a structure represented by a formula:

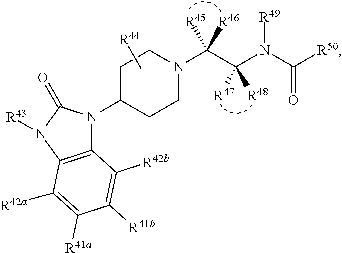

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, trifluoromethyl, carboxamido, alkylsulfonyl, an optionally substituted C1 to C6 alkyl, or an optionally substituted C3 to C6 cycloalkyl or R⁴⁵ and R⁴⁶, together with the intermediate carbon, comprise an optionally substituted C3 to C6 cycloalkyl; wherein each of R⁴⁷ and R⁴⁸ independently comprises hydrogen, trifluoromethyl, carboxamido, alkylsulfonyl, an optionally substituted C1 to C6 alkyl, or an optionally substituted C3 to C6 cycloalkyl or R⁴⁷ and R⁴⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting viral replication within the cell.

In a further aspect, the phospholipase D (PLD) inhibitor of the method for inhibiting HIV replication within a cell is a compound selected from: trans-diethylstilbestrol, resveratrol, honokiol, SCH420789, presqualene diphosphate, raloxifene, 4-hydroxy tamoxifen, 5-fluoro-2-indoyl des-chlorohalopemide, and halopemide, or a pharmaceutically acceptable salt, hydrate, solvate, or polymorph thereof, thereby inhibiting viral replication within the cell. In a further aspect, the method further comprises administering to the subject a non-PLD anti-HIV therapy. In a still further aspect, the method further comprises administering to the subject a non-PLD anti-HIV therapy selected from an M2 inhibitor, a neuraminidase inhibitor, and an interferon.

In a further aspect, the phospholipase D (PLD) inhibitor of the method for inhibiting HIV replication within a cell is a compound selected from:

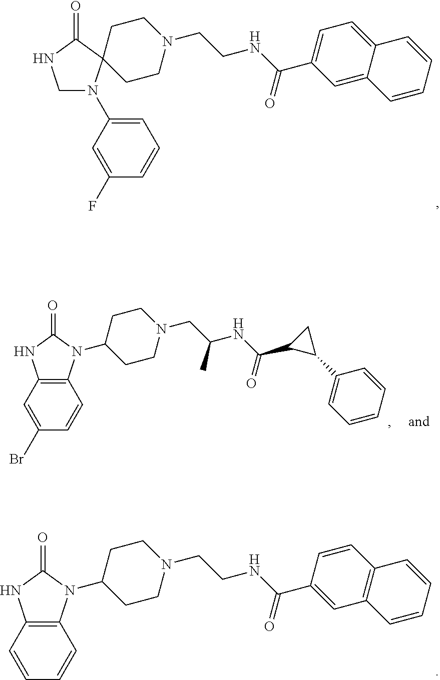

In a further aspect, the administering of the method comprises inhalation or oral administration. In a still further aspect, the administering of the method comprises intravenous or intra-arterial injection.

6. Inhibiting HIV Replication in a Cell by Contacting the Cell with a Compound that Binds PLD in a Non-Catalytic Domain

In one aspect, the invention relates to a method for inhibiting HIV replication within a cell, the method comprising the step of contacting the cell with an effective amount of a binding agent of phospholipase D (PLD), wherein the binding agent binds to at least one amino acid in a non-catalytic domain of PLD, thereby inhibiting HIV replication within the cell.

In a further aspect, the binding to PLD of the method is allosteric binding.

In a further aspect, the non-catalytic domain of the method comprises at least one amino acid residue in amino acids 1-505 of PLD1, or the homologous amino acids of PLD2. In a yet further aspect, the non-catalytic domain comprises at least one amino acid in amino acids 81-425 of PLD1, or the homologous amino acids of PLD2. In a still further aspect, the non-catalytic domain comprises at least one amino acid in amino acids 200-390 of PLD1, or the homologous amino acids of PLD2. In an even further aspect, the non-catalytic domain comprises at least one amino acid in amino acids 310-375, or the homologous amino acids of PLD2. In a still further aspect, the binding agent binds a domain comprising amino acids 310-375.

In one aspect, the invention relates to a method for inhibiting HIV replication within a cell, the method comprising contacting the cell with an effective amount of a binding agent of phospholipase D (PLD), wherein the binding agent binds to at least one amino acid residue in a binding domain comprising amino acids 1-505 of PLD1, or the homologous amino acids of PLD2, thereby inhibiting HIV replication within the cell.

In one aspect, the invention relates to a method for inhibiting HIV replication within a cell, the method comprising the step of contacting the cell with an effective amount of an allosteric binding agent of phospholipase D (PLD), thereby inhibiting HIV replication within the cell.

In a further aspect, the allosteric binding of the method occurs with at least one amino acid residue in amino acids 1-505 of PLD1, or the homologous amino acids of PLD2, thereby inhibiting viral entry into the cell. In a still further aspect, the allosteric binding of the method occurs with at least one amino acid residue in amino acids 81-425 of PLD1, or the homologous amino acids of PLD2. In a yet further aspect, the allosteric binding of the method occurs with at least one amino acid residue in amino acids 200-390 of claim 84, wherein PLD1, or the homologous amino acids of PLD2. In an even further aspect, the allosteric binding of the method occurs with at least one amino acid residue in amino acids 310-375, or the homologous amino acids of PLD2. In a still further aspect, the allosteric binding of the method occurs with a domain comprising amino acids 310-375.

In a further aspect, the binding of the foregoing methods modulates enzymatic activity. In a still further aspect, the binding of the foregoing methods is at a site directly or indirectly involved with protein-protein interaction.

In a further aspect, the cell of the foregoing methods is mammalian. In a still further aspect, the cell of the foregoing methods is human. In a yet further aspect, the cell of the foregoing methods cell has been isolated from a mammal prior to the contacting step. In an even further aspect, the contacting of the cell of the foregoing methods is via administration to a mammal.

In a further aspect, the agent of the foregoing methods for treating a subject for HIV infection is a compound having a structure represented by a formula:

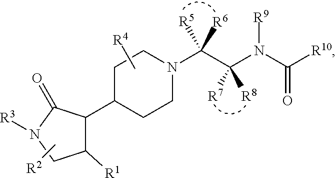

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, trifluoromethyl, carboxamido, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the agent of the foregoing methods for treating a subject for HIV infection is a compound having a structure represented by a formula:

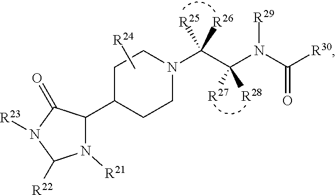

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, trifluoromethyl, carboxamido, alkylsulfonyl, an optionally substituted C1 to C6 alkyl, or an optionally substituted C3 to C6 cycloalkyl or R²⁵ and R²⁶, together with the intermediate carbon, comprise an optionally substituted C3 to C6 cycloalkyl; wherein each of R²⁷ and R²⁸ independently comprises hydrogen, trifluoromethyl, carboxamido, alkylsulfonyl, an optionally substituted C1 to C6 alkyl, or an optionally substituted C3 to C6 cycloalkyl or R²⁷ and R²⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the agent of the foregoing methods for treating a subject for HIV infection is a compound having a structure represented by a formula:

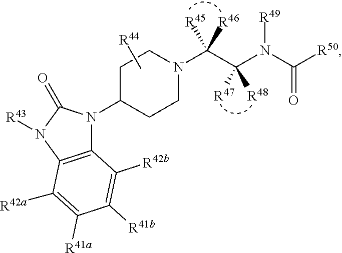

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, trifluoromethyl, carboxamido, alkoxy, thiol, alkylsulfonyl, an optionally substituted C1 to C6 alkyl, or an optionally substituted C3 to C6 cycloalkyl or R⁴⁵ and R⁴⁶, together with the intermediate carbon, comprise an optionally substituted C3 to C6 cycloalkyl; wherein each of R⁴⁷ and R⁴⁸ independently comprises hydrogen, trifluoromethyl, carboxamido, alkylsulfonyl, an optionally substituted C1 to C6 alkyl, or an optionally substituted C3 to C6 cycloalkyl or R⁴⁷ and R⁴⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the agent of the foregoing methods for treating a subject for HIV infection is a compound selected from: trans-diethylstilbestrol, resveratrol, honokiol, SCH420789, presqualene diphosphate, raloxifene, 4-hydroxy tamoxifen, 5-fluoro-2-indoyl des-chlorohalopemide, and halopemide, or a pharmaceutically acceptable salt, hydrate, solvate, or polymorph thereof, thereby treating the subject for HIV infection.

7. Inhibiting HIV Replication in a Cell by Contacting the Cell with a Compound that Binds PLD in a Catalytic Domain

In one aspect, the invention relates to a method for inhibiting HIV replication within a cell, the method comprising the step of contacting the cell with an effective amount of a binding agent of phospholipase D (PLD), wherein the binding agent binds to at least one amino acid in a catalytic domain of PLD, thereby inhibiting HIV replication within the cell.

In a further aspect, the binding to PLD of the method is allosteric binding. In a still further aspect, the binding to PLD of the method is orthosteric binding.

In a further aspect, the non-catalytic domain of the method comprises at least one amino acid residue in amino acids 463-928 of PLD1, or the homologous amino acids of PLD2.

In one aspect, the invention relates to a method for inhibiting HIV replication within a cell, the method comprising contacting the cell with an effective amount of a binding agent of phospholipase D (PLD), wherein the binding agent binds to at least one amino acid residue in a binding domain comprising amino acids 463-928 of PLD1, or the homologous amino acids of PLD2, thereby inhibiting HIV replication within the cell.

In one aspect, the invention relates to a method for inhibiting HIV replication within a cell, the method comprising the step of contacting the cell with an effective amount of an allosteric binding agent of phospholipase D (PLD), thereby inhibiting HIV replication within the cell. In a further aspect, the invention relates to a method for inhibiting HIV replication within a cell, the method comprising the step of contacting the cell with an effective amount of an orthosteric binding agent of phospholipase D (PLD), thereby inhibiting HIV replication within the cell.

In a further aspect, the allosteric binding of the method occurs with at least one amino acid residue in amino acids 463-928 of PLD1, or the homologous amino acids of PLD2, thereby inhibiting viral entry into the cell.

In a further aspect, the binding of the foregoing methods modulates enzymatic activity. In a still further aspect, the binding of the foregoing methods is at a site directly or indirectly involved with protein-protein interaction.

In a further aspect, the cell of the foregoing methods is mammalian. In a still further aspect, the cell of the foregoing methods is human. In a yet further aspect, the cell of the foregoing methods cell has been isolated from a mammal prior to the contacting step. In an even further aspect, the contacting of the cell of the foregoing methods is via administration to a mammal.

### I. METHODS OF INHIBITING HIV INTEGRATION IN CELLS

In one aspect, the method of use is directed to inhibition of HIV integration in cells. In a further aspect, the disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein which are usually applied in the treatment of the above mentioned pathological conditions.

1. Inhibiting HIV Integration in a Cell by Contacting the Cell with a PLD Inhibitor

In one aspect, the invention relates to a method for inhibiting HIV integration within a cell, the method comprising the step of contacting the cell with an effective amount of a phospholipase D (PLD) inhibitor, thereby inhibiting viral integration within the cell.

In a further aspect, the cell of the method is mammalian. In a still further aspect, the cell of the method is human. In a yet further aspect, the cell of the method has been isolated from a mammal prior to the contacting step.

In a further aspect, the PLD inhibited is PLD1. In a still further aspect, the phospholipase D (PLD) inhibitor is a PLD1-selective inhibitor. In a further aspect, the phospholipase D (PLD) inhibitor inhibits PLD1 response in an in vitro assay comprising a cultured cell-line. In a yet further aspect, the phospholipase D (PLD) inhibitor inhibits PLD1 response in Calu-1 cells.

In a further aspect, the PLD inhibited is PLD2. In a still further aspect, the phospholipase D (PLD) inhibitor is a PLD2-selective inhibitor. In a yet further aspect, the phospholipase D (PLD) inhibitor inhibits PLD2 response in HEK293gfpPLD2 cells.

In a further aspect, the phospholipase D (PLD) inhibitor inhibits in vitro PLD1 response. In a still further aspect, the phospholipase D (PLD) inhibitor has a PLD1 IC₅₀ of less than about 10 μM, of less than about 1 μM, of less than about 500 nM, of less than about 100 nM, of less than about 60 nM, or of less than about 20 nM. In a yet further aspect, the phospholipase D (PLD) inhibitor exhibits a PLD1:PLD2 inhibition ratio of at least about 2:1, of at least about 3:1, of at least about 5:1, of at least about 10:1, of at least about 20:1, of at least about 50:1, or of at least about 75:1.

In a further aspect, the phospholipase D (PLD) inhibitor inhibits in vitro PLD2 response. In a still further aspect, the phospholipase D (PLD) inhibitor has a PLD2 IC₅₀ of less than about 10 μM, of less than about 1 μM, of less than about 500 nM, of less than about 100 nM, of less than about 60 nM, or of less than about 20 nM. In a yet further aspect, the phospholipase D (PLD) inhibitor exhibits a PLD2:PLD1 inhibition ratio of at least about 2:1, of at least about 3:1, of at least about 5:1, of at least about 10:1, of at least about 20:1, of at least about 50:1, or of at least about 75:1.

In a further aspect, the phospholipase D (PLD) inhibitor of the method for inhibiting HIV replication within a cell is a compound having a structure represented by a formula:

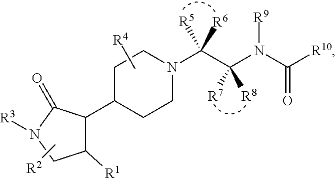

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, trifluoromethyl, carboxamido, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting viral replication within the cell.

In a further aspect, the phospholipase D (PLD) inhibitor of the method for inhibiting HIV replication within a cell is a compound having a structure represented by a formula:

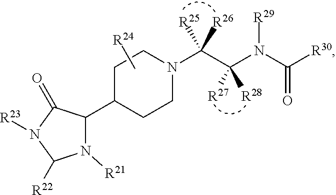

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, trifluoromethyl, carboxamido, alkylsulfonyl, an optionally substituted C1 to C6 alkyl, or an optionally substituted C3 to C6 cycloalkyl or R²⁵ and R²⁶, together with the intermediate carbon, comprise an optionally substituted C3 to C6 cycloalkyl; wherein each of R²⁷ and R²⁸ independently comprises hydrogen, trifluoromethyl, carboxamido, alkylsulfonyl, an optionally substituted C1 to C6 alkyl, or an optionally substituted C3 to C6 cycloalkyl or R²⁷ and R²⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting viral replication within the cell.

In a further aspect, the phospholipase D (PLD) inhibitor of the method for inhibiting HIV replication within a cell is a compound having a structure represented by a formula:

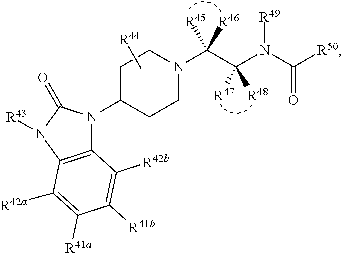

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, trifluoromethyl, carboxamido, alkylsulfonyl, an optionally substituted C1 to C6 alkyl, or an optionally substituted C3 to C6 cycloalkyl or R⁴⁵ and R⁴⁶, together with the intermediate carbon, comprise an optionally substituted C3 to C6 cycloalkyl; wherein each of R⁴⁷ and R⁴⁸ independently comprises hydrogen, trifluoromethyl, carboxamido, alkylsulfonyl, an optionally substituted C1 to C6 alkyl, or an optionally substituted C3 to C6 cycloalkyl or R⁴⁷ and R⁴⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby inhibiting viral replication within the cell.

In a further aspect, the phospholipase D (PLD) inhibitor of the method for inhibiting HIV replication within a cell is a compound selected from: trans-diethylstilbestrol, resveratrol, honokiol, SCH420789, presqualene diphosphate, raloxifene, 4-hydroxy tamoxifen, 5-fluoro-2-indoyl des-chlorohalopemide, and halopemide, or a pharmaceutically acceptable salt, hydrate, solvate, or polymorph thereof, thereby inhibiting viral replication within the cell. In a further aspect, the method further comprises administering to the subject a non-PLD anti-HIV therapy. In a still further aspect, the method further comprises administering to the subject a non-PLD anti-HIV therapy selected from an M2 inhibitor, a neuraminidase inhibitor, and an interferon.

In a further aspect, the phospholipase D (PLD) inhibitor of the method for inhibiting HIV replication within a cell is a compound selected from:

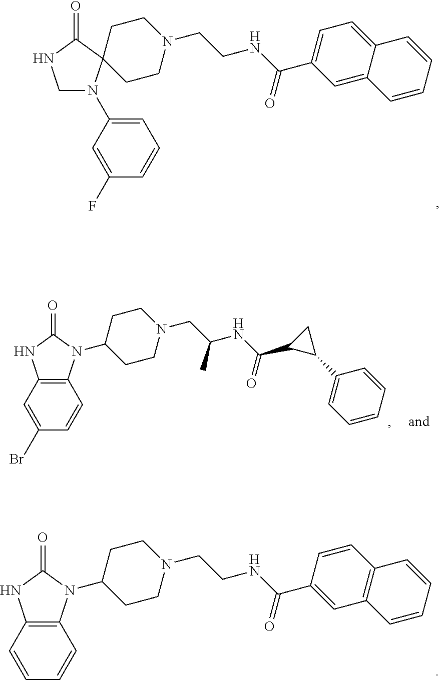

In a further aspect, the administering of the method comprises inhalation or oral administration. In a still further aspect, the administering of the method comprises intravenous or intra-arterial injection.

2. Inhibiting HIV Integration in a Cell by Contacting the Cell with a Compound that Binds PLD in a Non-Catalytic Domain

In one aspect, the invention relates to a method for inhibiting HIV integration within a cell, the method comprising the step of contacting the cell with an effective amount of a binding agent of phospholipase D (PLD), wherein the binding agent binds to at least one amino acid in a non-catalytic domain of PLD, thereby inhibiting HIV integration within the cell.

In a further aspect, the binding to PLD of the method is allosteric binding.

In a further aspect, the non-catalytic domain of the method comprises at least one amino acid residue in amino acids 1-505 of PLD1, or the homologous amino acids of PLD2. In a yet further aspect, the non-catalytic domain comprises at least one amino acid in amino acids 81-425 of PLD1, or the homologous amino acids of PLD2. In a still further aspect, the non-catalytic domain comprises at least one amino acid in amino acids 200-390 of PLD1, or the homologous amino acids of PLD2. In an even further aspect, the non-catalytic domain comprises at least one amino acid in amino acids 310-375, or the homologous amino acids of PLD2. In a still further aspect, the binding agent binds a domain comprising amino acids 310-375.

In one aspect, the invention relates to a method for inhibiting HIV integration within a cell, the method comprising the step of contacting the cell with an effective amount of a binding agent of phospholipase D (PLD), wherein the binding agent binds to at least one amino acid residue in a binding domain comprising amino acids 1-505 of PLD1, or the homologous amino acids of PLD2, thereby inhibiting HIV integration within the cell.

In one aspect, the invention relates to a method for inhibiting HIV integration within a cell, the method comprising the step of contacting the cell with an effective amount of an allosteric binding agent of phospholipase D (PLD), thereby inhibiting HIV integration within the cell.

In a further aspect, the allosteric binding of the method occurs with at least one amino acid residue in amino acids 1-505 of PLD1, or the homologous amino acids of PLD2, thereby inhibiting viral entry into the cell. In a still further aspect, the allosteric binding of the method occurs with at least one amino acid residue in amino acids 81-425 of PLD1, or the homologous amino acids of PLD2. In a yet further aspect, the allosteric binding of the method occurs with at least one amino acid residue in amino acids 200-390 of claim 84, wherein PLD1, or the homologous amino acids of PLD2. In an even further aspect, the allosteric binding of the method occurs with at least one amino acid residue in amino acids 310-375, or the homologous amino acids of PLD2. In a still further aspect, the allosteric binding of the method occurs with a domain comprising amino acids 310-375.

In a further aspect, the binding of the foregoing methods modulates enzymatic activity. In a still further aspect, the binding of the foregoing methods is at a site directly or indirectly involved with protein-protein interaction.

In a further aspect, the cell of the foregoing methods is mammalian. In a still further aspect, the cell of the foregoing methods is human. In a yet further aspect, the cell of the foregoing methods cell has been isolated from a mammal prior to the contacting step. In an even further aspect, the contacting of the cell of the foregoing methods is via administration to a mammal.

In a further aspect, the agent of the foregoing methods for treating a subject for HIV infection is a compound having a structure represented by a formula:

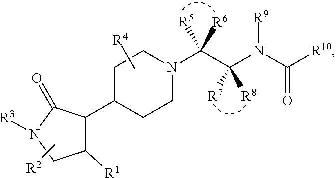

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, trifluoromethyl, carboxamido, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, trifluoromethyl, carboxamido, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the agent of the foregoing methods for treating a subject for HIV infection is a compound having a structure represented by a formula:

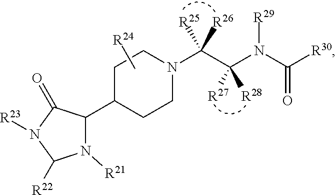

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, trifluoromethyl, carboxamido, alkylsulfonyl, an optionally substituted C1 to C6 alkyl, or an optionally substituted C3 to C6 cycloalkyl or R²⁵ and R²⁶, together with the intermediate carbon, comprise an optionally substituted C3 to C6 cycloalkyl; wherein each of R²⁷ and R²⁸ independently comprises hydrogen, trifluoromethyl, carboxamido, alkylsulfonyl, an optionally substituted C1 to C6 alkyl, or an optionally substituted C3 to C6 cycloalkyl or R²⁷ and R²⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the agent of the foregoing methods for treating a subject for HIV infection is a compound having a structure represented by a formula:

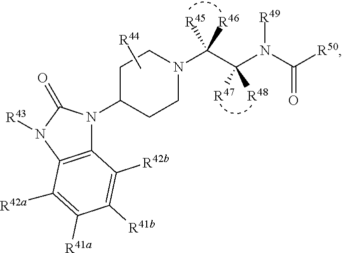

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, trifluoromethyl, carboxamido, alkoxy, thiol, alkylsulfonyl, an optionally substituted C1 to C6 alkyl, or an optionally substituted C3 to C6 cycloalkyl or R⁴⁵ and R⁴⁶, together with the intermediate carbon, comprise an optionally substituted C3 to C6 cycloalkyl; wherein each of R⁴⁷ and R⁴⁸ independently comprises hydrogen, trifluoromethyl, carboxamido, alkylsulfonyl, an optionally substituted C1 to C6 alkyl, or an optionally substituted C3 to C6 cycloalkyl or R⁴⁷ and R⁴⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby treating the subject for HIV infection.

In a further aspect, the agent of the foregoing methods for treating a subject for HIV infection is a compound selected from: trans-diethylstilbestrol, resveratrol, honokiol, SCH420789, presqualene diphosphate, raloxifene, 4-hydroxy tamoxifen, 5-fluoro-2-indoyl des-chlorohalopemide, and halopemide, or a pharmaceutically acceptable salt, hydrate, solvate, or polymorph thereof, thereby treating the subject for HIV infection.

3. Inhibiting HIV Integration in a Cell by Contacting the Cell with a Compound that Binds PLD in a Catalytic Domain

In one aspect, the invention relates to a method for inhibiting HIV integration within a cell, the method comprising the step of contacting the cell with an effective amount of a binding agent of phospholipase D (PLD), wherein the binding agent binds to at least one amino acid in a catalytic domain of PLD, thereby inhibiting HIV integration within the cell.

In a further aspect, the binding to PLD of the method is allosteric binding. In a still further aspect, the binding to PLD of the method is orthosteric binding.

In a further aspect, the non-catalytic domain of the method comprises at least one amino acid residue in amino acids 463-928 of PLD1, or the homologous amino acids of PLD2.

In one aspect, the invention relates to a method for inhibiting HIV integration within a cell, the method comprising the step of contacting the cell with an effective amount of a binding agent of phospholipase D (PLD), wherein the binding agent binds to at least one amino acid residue in a binding domain comprising amino acids 463-928 of PLD1, or the homologous amino acids of PLD2, thereby inhibiting HIV integration within the cell.

In one aspect, the invention relates to a method for inhibiting HIV integration within a cell, the method comprising the step of contacting the cell with an effective amount of an allosteric binding agent of phospholipase D (PLD), thereby inhibiting HIV integration within the cell. In a further aspect, the invention relates to a method for inhibiting HIV integration within a cell, the method comprising the step of contacting the cell with an effective amount of an orthosteric binding agent of phospholipase D (PLD), thereby inhibiting HIV integration within the cell.

In a further aspect, the allosteric binding of the method occurs with at least one amino acid residue in amino acids 463-928 of PLD1, or the homologous amino acids of PLD2, thereby inhibiting viral entry into the cell.

In a further aspect, the binding of the foregoing methods modulates enzymatic activity. In a still further aspect, the binding of the foregoing methods is at a site directly or indirectly involved with protein-protein interaction.

In a further aspect, the cell of the foregoing methods is mammalian. In a still further aspect, the cell of the foregoing methods is human. In a yet further aspect, the cell of the foregoing methods cell has been isolated from a mammal prior to the contacting step. In an even further aspect, the contacting of the cell of the foregoing methods is via administration to a mammal.

### J. METHODS OF DECREASING HIV VIRAL LOAD IN CELLS

In one aspect, the method of use is directed to decreasing HIV viral load in cells. In a further aspect, the disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein which are usually applied in the treatment of the above mentioned pathological conditions.

1. Decreasing HIV Viral Load in a Cell by Contacting the Cell with a 1-oxo-2,8-diazaspiro[4.5]decanyl analog

In one aspect, the invention relates to a method for decreasing HIV viral load in at least one cell, comprising the step of contacting the at least one cell with an effective amount of at least one compound having a structure represented by a formula:

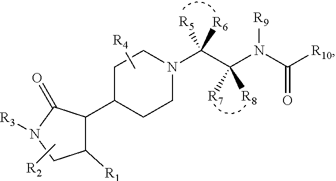

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby decreasing HIV viral load in at least one cell.

In a further aspect, the compound of the method has a structure represented by a formula:

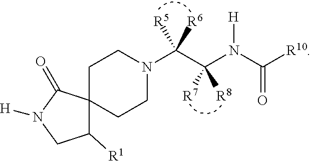

In a further aspect, the cell of the method is mammalian. In a still further aspect, the cell of the method is human. In a yet further aspect, the cell of the method has been isolated from a mammal prior to the contacting step.

In a further aspect, the cell of the method is an activated CD4⁺ T-lymphocyte. In a still further aspect, the cell is a resting or memory T-cell. In yet a further aspect, the cell is a tissue macrophage. In an even further aspect, the tissue macrophage is a brain macrophage. In a still further aspect, the tissue macrophage is a microglial cell.

In a further aspect, contacting is via administration to a subject. In a still further aspect, the subject has been diagnosed with a need for inhibiting HIV replication prior to the administering step. In yet a further aspect, the subject has been diagnosed with a need for treatment of HIV related to HIV replication prior to the administering step.

2. Decreasing HIV Viral Load in a Cell by Contacting the Cell with a 4-oxo-1,3,8-triazaspiro[4.5]decanyl analog

In one aspect, the invention relates to a method for decreasing HIV viral load in at least one cell, comprising the step of contacting the at least one cell with an effective amount of at least one compound having a structure represented by a formula:

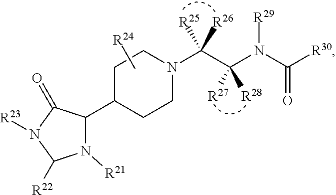

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby decreasing HIV viral load in at least one cell.

In a further aspect, the compound of the method has a structure represented by a formula:

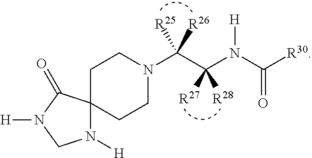

In a further aspect, the compound of the method is:

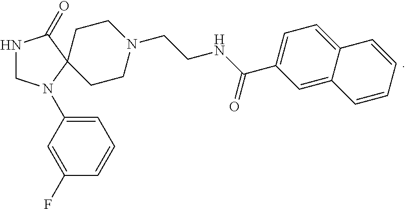 [SMILES: O=C(NCCN1CCC2(CC1)C(=O)NCN2c1cccc(F)c1)c1ccc2ccccc2c1]

In a further aspect, the cell of the method is mammalian. In a still further aspect, the cell of the method is human. In a yet further aspect, the cell of the method has been isolated from a mammal prior to the contacting step.

In a further aspect, the cell of the method is an activated CD4⁺ T-lymphocyte. In a still further aspect, the cell is a resting or memory T-cell. In yet a further aspect, the cell is a tissue macrophage. In an even further aspect, the tissue macrophage is a brain macrophage. In a still further aspect, the tissue macrophage is a microglial cell.

In a further aspect, contacting is via administration to a subject. In a still further aspect, the subject has been diagnosed with a need for inhibiting HIV replication prior to the administering step. In yet a further aspect, the subject has been diagnosed with a need for treatment of HIV related to HIV replication prior to the administering step.

3. Decreasing HIV Viral Load in a Cell by Contacting the Cell with a Substituted 2-oxo-2,3-dihydro-1H-benzo[d]imidazol-1-yl analog

In one aspect, the invention relates to a method for decreasing HIV viral load in at least one cell, comprising the step of contacting the at least one cell with an effective amount of at least one compound having a structure represented by a formula:

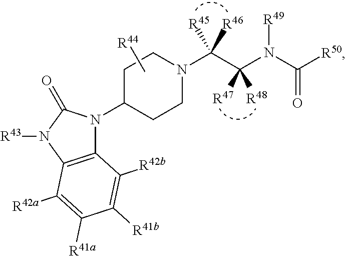

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby decreasing HIV viral load in at least one cell.

In a further aspect, the compound of the method has a structure represented by a formula:

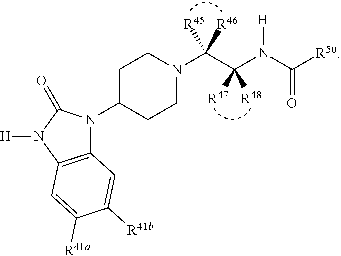

In a further aspect, the compound of the method is:

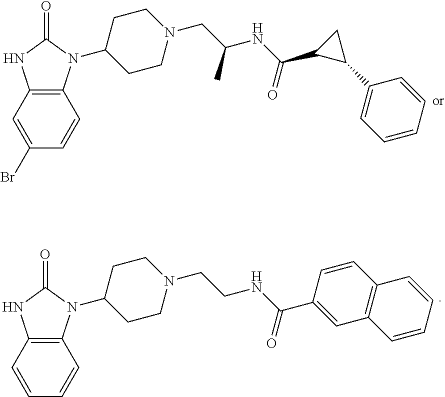

In a further aspect, the cell of the method is mammalian. In a still further aspect, the cell of the method is human. In a yet further aspect, the cell of the method has been isolated from a mammal prior to the contacting step.

In a further aspect, the cell of the method is an activated CD4⁺ T-lymphocyte. In a still further aspect, the cell is a resting or memory T-cell. In yet a further aspect, the cell is a tissue macrophage. In an even further aspect, the tissue macrophage is a brain macrophage. In a still further aspect, the tissue macrophage is a microglial cell.

In a further aspect, contacting is via administration to a subject. In a still further aspect, the subject has been diagnosed with a need for inhibiting HIV replication prior to the administering step. In yet a further aspect, the subject has been diagnosed with a need for treatment of HIV related to HIV replication prior to the administering step.

4. Decreasing HIV Viral Load in a Cell by Contacting the Cell with a Selected Compound

In one aspect, the invention relates to a method for decreasing HIV viral load in at least one cell, comprising the step of contacting the at least one cell with an effective amount of at least one compound having a structure represented by a formula: trans-diethylstilbestrol, resveratrol, honokiol, SCH420789, presqualene diphosphate, raloxifene, 4-hydroxytamoxifen, 5-fluoro-2-indoyl des-chlorohalopemide, and halopemide, or a pharmaceutically acceptable salt, hydrate, solvate, or polymorph thereof, thereby decreasing HIV viral load in at least one cell.

In one aspect, the invention relates to a method for decreasing HIV viral load in at least one cell, comprising the step of contacting the at least one cell with an effective amount of at least one compound having a structure represented by a formula:

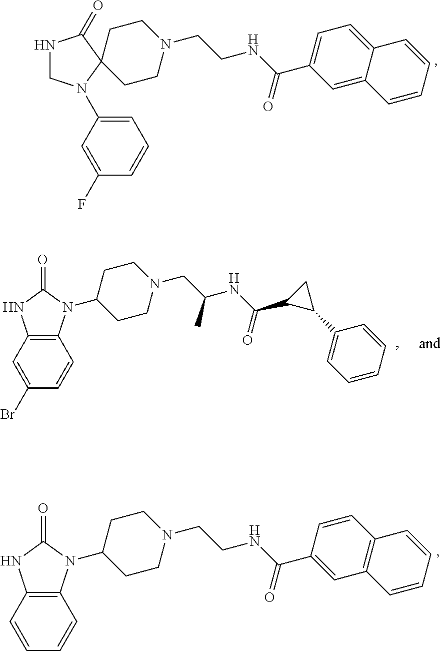

or a pharmaceutically acceptable salt, hydrate, solvate, or polymorph thereof, thereby decreasing HIV viral load in at least one cell.

In a further aspect, the cell of the method is mammalian. In a still further aspect, the cell of the method is human. In a yet further aspect, the cell of the method has been isolated from a mammal prior to the contacting step.

In a further aspect, the cell of the method is an activated CD4⁺ T-lymphocyte. In a still further aspect, the cell is a resting or memory T-cell. In yet a further aspect, the cell is a tissue macrophage. In an even further aspect, the tissue macrophage is a brain macrophage. In a still further aspect, the tissue macrophage is a microglial cell.

In a further aspect, contacting is via administration to a subject. In a still further aspect, the subject has been diagnosed with a need for inhibiting HIV replication prior to the administering step. In yet a further aspect, the subject has been diagnosed with a need for treatment of HIV related to HIV replication prior to the administering step.

### K. METHODS OF DECREASING NUCLEOTIDE POOLS IN CELLS

In one aspect, the method of use is directed to decreasing nucleotide pools in cells. In a further aspect, the disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein which are usually applied in the treatment of the above mentioned pathological conditions.

1. Decreasing Nucleotide Pools in Cells by Contacting the Cell with a 1-oxo-2,8-diazaspiro[4.5]decanyl analog

In one aspect, the invention relates to a method for decreasing nucleotide pools in at least one cell, comprising the step of contacting the at least one cell with an effective amount of at least one compound having a structure represented by a formula:

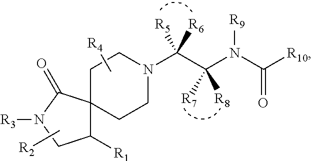

wherein each ----- independently comprises an optional covalent bond; wherein R¹ is an optionally substituted C3 to C9 organic residue selected from aryl, heteroaryl, cycloalkyl, heterocycloalkyl, cycloalkenyl, and heterocycloalkenyl; wherein R² comprises three substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R³ comprises hydrogen, an optionally substituted C1 to C6 alkyl, an optionally substituted C3 to C6 cycloalkyl, or a hydrolysable residue; wherein R⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁵ and R⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁷ and R⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁹ comprises hydrogen, an optionally substituted C1 to C6 alkyl, an optionally substituted C3 to C6 cycloalkyl, or a hydrolysable residue; wherein R¹⁰ comprises an optionally substituted C1 to C12 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby decreasing nucleotide pools in at least one cell.

In a further aspect, the compound of the method has a structure represented by a formula:

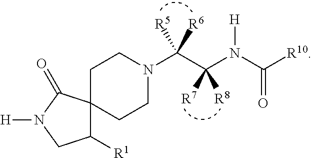

In a further aspect, the cell of the method is mammalian. In a still further aspect, the cell of the method is human. In a yet further aspect, the cell of the method has been isolated from a mammal prior to the contacting step.

In a further aspect, the cell of the method is infected with an HIV virus.

In a further aspect, the cell of the method is an activated CD4⁺ T-lymphocyte. In a still further aspect, the cell is a resting or memory T-cell. In yet a further aspect, the cell is a tissue macrophage. In an even further aspect, the tissue macrophage is a brain macrophage. In a still further aspect, the tissue macrophage is a microglial cell.

In a further aspect, contacting is via administration to a subject. In a still further aspect, the subject has been diagnosed with a need for inhibiting HIV replication prior to the administering step. In yet a further aspect, the subject has been diagnosed with a need for treatment of HIV related to HIV replication prior to the administering step.

2. Decreasing Nucleotide Pools in Cells by Contacting the Cell with a 4-oxo-1,3,8-triazaspiro[4.5]decanyl analog

In one aspect, the invention relates to a method for decreasing nucleotide pools in at least one cell, comprising the step of contacting the at least one cell with an effective amount of at least one compound having a structure represented by a formula:

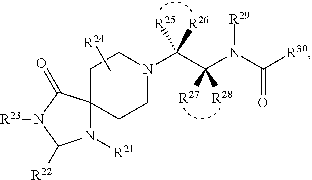

wherein each ----- independently comprises an optional covalent bond; wherein R²¹ is an optionally substituted C3 to C9 organic residue selected from aryl, heteroaryl, cycloalkyl, heterocycloalkyl, cycloalkenyl, and heterocycloalkenyl; wherein R²² comprises two substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R²³ comprises hydrogen, an optionally substituted C1 to C6 alkyl, an optionally substituted C3 to C6 cycloalkyl, or a hydrolysable residue; wherein R²⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R²⁵ and R²⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R²⁷ and R²⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R²⁹ comprises hydrogen, an optionally substituted C1 to C6 alkyl, an optionally substituted C3 to C6 cycloalkyl, or a hydrolysable residue; wherein R³⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby decreasing nucleotide pools in at least one cell.

In a further aspect, the compound of the method has a structure represented by a formula:

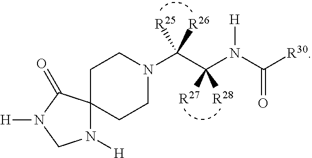

In a further aspect, the compound of the method is:

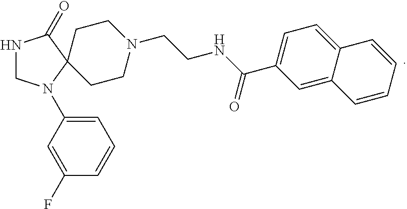 [SMILES: O=C(NCCN1CCC2(CC1)C(=O)NCN2c1cccc(F)c1)c1ccc2ccccc2c1]

In a further aspect, the cell of the method is mammalian. In a still further aspect, the cell of the method is human. In a yet further aspect, the cell of the method has been isolated from a mammal prior to the contacting step.

In a further aspect, the cell of the method is infected with an HIV virus.

In a further aspect, the cell of the method is an activated CD4⁺ T-lymphocyte. In a still further aspect, the cell is a resting or memory T-cell. In yet a further aspect, the cell is a tissue macrophage. In an even further aspect, the tissue macrophage is a brain macrophage. In a still further aspect, the tissue macrophage is a microglial cell.

In a further aspect, contacting is via administration to a subject. In a still further aspect, the subject has been diagnosed with a need for inhibiting HIV replication prior to the administering step. In yet a further aspect, the subject has been diagnosed with a need for treatment of HIV related to HIV replication prior to the administering step.

3. Decreasing Nucleotide Pools in Cells by Contacting the Cell with Substituted 2-oxo-2,3-dihydro-1H-benzo[d]imidazol-1-yl analog

In one aspect, the invention relates to a method for decreasing nucleotide pools in at least one cell, comprising the step of contacting the at least one cell with an effective amount of at least one compound having a structure represented by a formula:

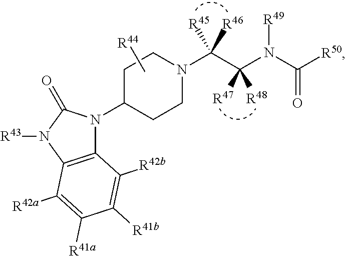

wherein each ----- independently comprises an optional covalent bond; wherein each of R^{41a} and R^{41b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R^{42a} and R^{42b} is independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein R⁴³ comprises hydrogen, an optionally substituted C1 to C6 alkyl, an optionally substituted C3 to C6 cycloalkyl, or a hydrolysable residue; wherein R⁴⁴ comprises eight substituents independently selected from hydrogen, halide, hydroxyl, trifluoromethyl, amino, cyano, nitro, azide, carboxamido, alkoxy, thiol, alkylsulfonyl, and an optionally substituted C1 to C6 organic residue; wherein each of R⁴⁵ and R⁴⁶ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁵ and R⁶, together with the intermediate carbon, comprise an optionally substituted C3 to C6 cycloalkyl; wherein each of R⁴⁷ and R⁴⁸ independently comprises hydrogen, halide, hydroxyl, trifluoromethyl, amino, cyano, nitro, azide, carboxamido, alkoxy, thiol, alkylsulfonyl, an optionally substituted C1 to C6 alkyl, or an optionally substituted C3 to C6 cycloalkyl or R⁷ and R⁸, together with the intermediate carbon, comprise an optionally substituted C3 to C6 cycloalkyl; wherein R⁴⁹ comprises hydrogen, an optionally substituted C1 to C6 alkyl, an optionally substituted C3 to C6 cycloalkyl, or a hydrolysable residue; wherein R⁵⁰ comprises an optionally substituted C1 to C16 organic residue selected from alkyl, aryl, heteroaryl, cycloalkyl, heterocycloalkyl, cycloalkenyl, and heterocycloalkenyl, or a pharmaceutically acceptable salt, hydrate, solvate, or polymorph thereof, thereby decreasing nucleotide pools in at least one cell.

In a further aspect, the compound of the method has a structure represented by a formula:

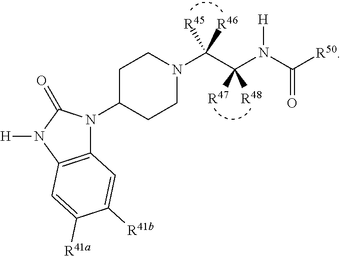

In a further aspect, the compound of the method is:

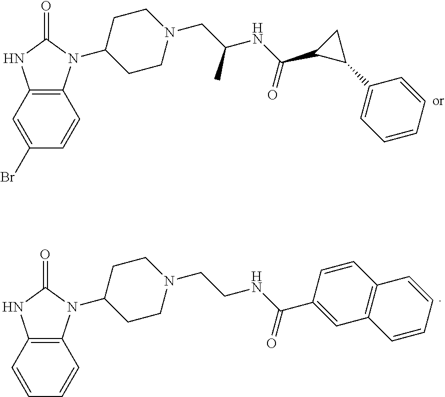

In a further aspect, the cell of the method is mammalian. In a still further aspect, the cell of the method is human. In a yet further aspect, the cell of the method has been isolated from a mammal prior to the contacting step.

In a further aspect, the cell of the method is infected with an HIV virus.

In a further aspect, the cell of the method is an activated CD4⁺ T-lymphocyte. In a still further aspect, the cell is a resting or memory T-cell. In yet a further aspect, the cell is a tissue macrophage. In an even further aspect, the tissue macrophage is a brain macrophage. In a still further aspect, the tissue macrophage is a microglial cell.

In a further aspect, contacting is via administration to a subject. In a still further aspect, the subject has been diagnosed with a need for inhibiting HIV replication prior to the administering step. In yet a further aspect, the subject has been diagnosed with a need for treatment of HIV related to HIV replication prior to the administering step.

4. Decreasing Nucleotide Pools in Cells by Contacting the Cell with a Selected Compound

In one aspect, the invention relates to a method for decreasing nucleotide pools in at least one cell, comprising the step of contacting the at least one cell with an effective amount of at least one compound having a structure represented by a formula: trans-diethylstilbestrol, resveratrol, honokiol, SCH420789, presqualene diphosphate, raloxifene, 4-hydroxytamoxifen, 5-fluoro-2-indoyl des-chlorohalopemide, and halopemide, or a pharmaceutically acceptable salt, hydrate, solvate, or polymorph thereof, thereby decreasing nucleotide pools in at least one cell.

In one aspect, the invention relates to a method for decreasing nucleotide pools in at least one cell, comprising the step of contacting the at least one cell with an effective amount of at least one compound having a structure represented by a formula:

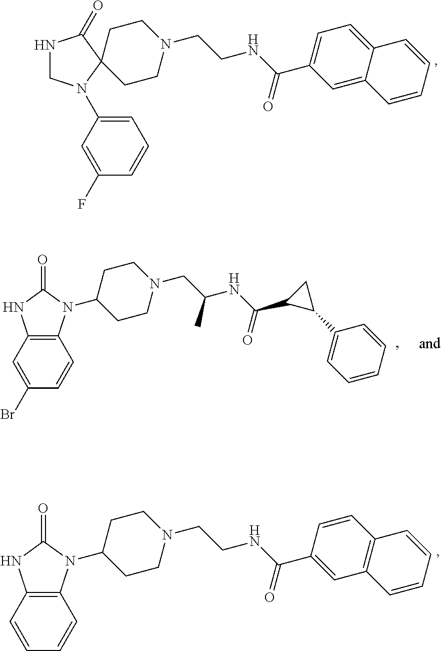

or a pharmaceutically acceptable salt, hydrate, solvate, or polymorph thereof, thereby decreasing nucleotide pools in at least one cell.

In a further aspect, the cell of the method is mammalian. In a still further aspect, the cell of the method is human. In a yet further aspect, the cell of the method has been isolated from a mammal prior to the contacting step.

In a further aspect, the cell of the method is infected with an HIV virus.

In a further aspect, the cell of the method is an activated CD4⁺ T-lymphocyte. In a still further aspect, the cell is a resting or memory T-cell. In yet a further aspect, the cell is a tissue macrophage. In an even further aspect, the tissue macrophage is a brain macrophage. In a still further aspect, the tissue macrophage is a microglial cell.

In a further aspect, contacting is via administration to a subject. In a still further aspect, the subject has been diagnosed with a need for inhibiting HIV replication prior to the administering step. In yet a further aspect, the subject has been diagnosed with a need for treatment of HIV related to HIV replication prior to the administering step.

### L. USES OF THE DISCLOSED COMPOUNDS

In a further aspect, the invention relates to use of at least one disclosed compound in the manufacture of a medicament for the treatment of an HIV infection. In a further aspect, the use is in the manufacture of a medicament for the treatment of an HIV infection in a mammal.

In a further aspect, the medicament further comprises a non-PLD anti-HIV therapy.

In a further aspect, the medicament is formulated for inhalation or oral administration. In a still further aspect, the medicament is formulated for intravenous or intra-arterial injection.

It is understood that the disclosed uses can be employed in connection with the disclosed compounds, methods, compositions, and kits.

### M. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Methods

a. Materials and Reagents

1-BuOH-d₁₀ was purchased from CDN Isotopes (Quebec Canada) and 1-BuOH was purchased from EM Science (OmniSolv, Gibbstown, N.J.). All solvents used for extraction or mass spectrometry were of HPLC grade or better, purchased from EMD Chemicals (NJ, USA). UDP, R59949 and Ro 32-0432 were purchased from Sigma. [9,10-³H]Oleic acid (5.0 Ci/mmol) was purchased from PerkinElmer Life Sciences (Boston, Mass.); Silica gel 60 Å TLC plates, 20×20 cm, were purchased from Whatman (Clifton, N.J.); lipid standards, 32:0 phosphatidyl methanol (PtdMeOH) and 24:0 diacylglycerol (DAG), were purchased from Avanti Polar Lipids (Alabaster, Ala.). Phorbol 12-myristate 13-acetate (PMA) was purchased from Calbiochem (San Diego, Calif.). Mass spectrometry and siRNA results were analyzed by either Student's t-test or ANOVA to assess changes between conditions in replicate experiments.

b. PLD Inhibitor Compounds

The representative PLD inhibitor compounds used in the various studies described herein below are shown below in Table 1, and were synthesized as previously described (Lavieri et al. (2010) J. Med. Chem. 53 6709; and Scott, S. A., et al. (2009) Nat. Chem. Biol. 5:108-117). The inhibitor activity of these representative PLD inhibitors is provided in Table 2.

TABLE 1

| | | Reference | |
|---|---|---|---|
| No. | Structure | Codes | Chemical Name |
| 1 | 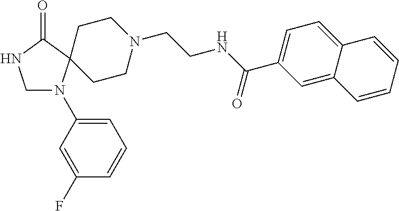 [SMILES: O=C(NCCN1CCC2(CC1)C(=O)NCN2c1cccc(F)c1)c1ccc2ccccc2c1] | JWJ; VU0364739 | N-(2-(1-(3-fluorophenyl)- 4-oxo-1,3,8-triazaspiro [4.5]decan-8-yl)ethyl)-2- naphthamide |
| 2 | 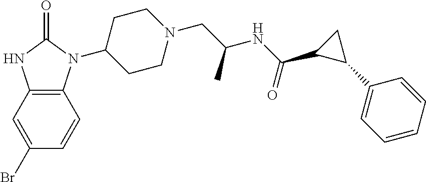 | EVJ; VU0359595 | (1R,2S)-N-((S)-1-(4-(5- bromo-2-oxo-2,3- dihydro-1H- benzo[d]imidazol-1- yl)piperidin-1- yl)propan-2-yl)-2- phenylcyclopropane carboxamide |
| 3 | 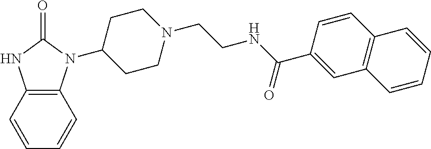 [SMILES: O=C(NCCN1CCC(n2c(=O)[nH]c3ccccc32)CC1)c1ccc2ccccc2c1] | 5WO; VU0155056 | N-(2-(4-(2-oxo-2,3- dihydro-1H- benzo[d]imidazol-1- yl)piperidin-1-yl)ethyl)- 2-naphthamide |

TABLE 2

| | Reference | PLD1* | PLD2* | PLD1** | PLD2** |
|---|---|---|---|---|---|
| No. | Codes | (IC₅₀, nM) | (IC₅₀, nM) | (IC₅₀, nM) | (IC₅₀, nM) |
| 1 | JWJ; | 1,500 | 20 | 7,400 | 100 |
| | VU0364739 | | | | |
| 2 | EVJ; | 3.7 | 6,400 | 15 | 1,100 |
| | VU0359595 | | | | |
| 3 | 5WO; | 21 | 380 | 80 | 240 |
| | VU0155056 | | | | |

*Cellular assay
**In vitro enzyme assay

c. Cell Culture

THP-1 and primary macrophages were grown in a variety of tissue culture media supplemented with 10% fetal bovine serum (FBS) and 1% Antibiotic-Antimycotic (AA). As appropriate G418 was used as a selection agent in a 37° C. humidified atmosphere with 5% CO₂.

d. PLD Endogenous Assay Using Deuterated 1-BuOH and MS

MS-based PLD endogenous assay was performed essentially as previously described (Brown, H. A. et al. Methods Enzymol. (2007) 434:49-87). Briefly, cells were seeded into 6-well tissue culture plates and designed experiments were carried out in the presence or absence of 0.3% 1-BuOH-d₁₀ for the desired times under specified experimental conditions. At the end of the stimulation, plates were placed on ice and media aspirated. Glycerophospholipids were extracted by the modified Bligh and Dyer procedure as described above and analyzed in the same manner as the glycerophospholipids. For these samples 100 ng 32:0 PtdMeOH was used as an internal standard to quantitate the PtdBuOH-d₉ species. As PtdBuOH is the unique product of PLD activity, its fatty acids and molecular species composition reflects that of PLD's substrate.

e. PLD Endogenous Assay Using Radioisotopes

PLD activity was assessed by measuring accumulation of the PLD activity marker phosphatidylbutanol (PtdBuOH) that is generated in the presence of 1-BuOH by a transphosphatidylation reaction. Briefly, cells were labeled with [³H] oleic acid, 10 μCi/ml, and incubated overnight. Cells were stimulated with UDP (at various concentrations) in the presence of 0.3% 1-BuOH for 30 min. Global lipids were extracted and separated as previously described. Lipids were then imaged using a Phosphoimager tritium screen for 72 hour and stained with iodine to visualize standards. PtdBuOH and PA were quantitated using Quantity One software (Biorad).

f. Glycerophospholipid Extraction for Mass Spectrometry

Glycerophospholipids were extracted using a modified Bligh and Dyer procedure. Briefly, cells were plated in 6-well tissue culture plates at 300,000 cells/well 48 hours prior to experiment in complete growth medium. Following treatment media was removed and cells were scraped in 0.1 N HCl:MeOH (1:1) suspension was then transferred to cold 1.5 ml Eppendorf tubes and vortexed with 400 μl of cold CHCl₃ for 1 min. The extraction proceeded with centrifugation (5 min, 4° C., 18,000×g) to separate the two phases. Lower organic layer was collected, lipid internal standards added and solvent evaporated. The resulting lipid film was dissolved in 100 μl of isopropanol:hexane:100 mM NH₄COOH(aq) 58:40:2 for LC-MS analysis.

g. Diacylglycerol Isolation and Detection

DAG isolation from total phospholipids extracts was achieved. Briefly, after phospholipid extraction by modified Bligh and Dyer procedure, each sample was applied to a glass Pasteur pipette column plugged with glass wool and packed with a 6 cm bed of silica gel 60 Å equilibrated with 10 mL of eluent (65:35:0.7 CHCl₃:CH₃OH:H₂O). DAG molecular species were recovered in the first 3 mL of eluent, and solvents were evaporated in a vacuum centrifuge. Samples were dissolved in 65 μL of 9:1 CH₃OH:CHCl₃ containing 5 μL of 100 mM CH₃COONa and analyzed by mass spectrometry as sodium adducts. For quantitation 100 ng of 24:0 DAG was used as an internal standard.

h. Lipid Mass Spectrometry

Glycerophospholipids were analyzed on an Applied Biosystems/MDS SCIEX 4000 Q TRAP hybrid triple quadrupole/linear ion trap mass spectrometer (Applied Biosystems, Foster City, Calif., USA) and a Shimadzu high pressure liquid chromatography system with a Phenomenex Luna Silica column (2×250 mm, 5-μm particle size) using a gradient elution as previously described. The identification of the individual species, achieved by LC/MS/MS, was based on their chromatographic and mass spectral characteristics. This analysis allows identification of the two fatty acid moieties but does not determine their position on the glycerol backbone (sn-1 versus sn-2). Quantification of glycerophospholipids was achieved by the use of an LC-MS technique employing synthetic odd-carbon diacyl and lysophospholipid standards.

For diacylglycerol species mass spectral analysis was performed on a Finnigan TSQ Quantum triple quadrupole mass spectrometer (Thermo Finnigan, San Jose, Calif.) equipped with a Harvard Apparatus syringe pump and an electrospray source. Samples were analyzed at an infusion rate of 10 μL/min. DAG samples were analyzed in positive mode with the scan range from m/z 400-900. Data were collected with the Xcalibur software package (Thermo Finnigan) and analyzed with software developed in the Brown laboratory.

1. siRNA Protein Knock-Down

Cells were plated at approximately 2.2×10⁵ cells/well on 6-well plates with growth medium (DMEM, 10% FBS) 24 hours before transfection. Cells ˜50% confluent at time of transfection. On-Target Plus SMART pools (O-TPSp) of siRNA (Dharmacon) of each target gene were transfected (100 nM siRNA/well) using Dharmafect 1 (Dharmacon) according to manufacturer's protocol. After 18 hours, the transfection medium was replaced with growth medium. Activity assays were carried out 72 hours post siRNA transfection. PLD activity was then measured using either TLC or MS Endogenous PLD activity assays and protein knock-down was confirmed for each target with western blotting. Western analysis was performed to confirm the knockdown efficiency of each target gene by siRNA. Antibodies for each protein used.

2. Pharmacokinetics of PLD Inhibitor Administered in a Mouse Model

The pharmacokinetic behavior of a representative disclosed PLD inhibitor of the present invention was assessed following intraperitoneal administration in a rat model. Briefly, mice were administered a single dose of a representative PLD inhibitor, either VUO364739 or VU359595 (10 mg/kg) by either intravenous injection or by oral administration. Plasma samples and tissue samples (brain) were isolated at various time points following euthanizing animal. The samples were flash frozen and stored at −80° C. Analysis was carried out by LC-MS/MS following extraction. The data are shown in Table 3 below.

TABLE 3

| | Plasma Protein | IV (pharmacokinetics) | | | | PO (plasma and brain levels) | | | |
|---|---|---|---|---|---|---|---|---|---|
| | Binding | Dose | CI | t_{1/2} | V_{dss} | Dose | Plasma | Brain | Brain: |
| Cmpd. | (% bound) | (mg/kg) | (mL/min/kg) | (h) | (L/kg) | (mg/kg) | (ng/kg) | (ng/kg) | Plasma |
| EVJ | 97.9 | 1 | 61.5 | 1.52 | 8.1 | 10 | 39.9 | 29 | 0.73 |
| JWJ | 98.1 | 1 | 60.7 | 0.78 | 4.7 | 10 | 29 | BLQ | BLQ |

3. HIV-1 Replication in Macrophages

Approximately 5×10⁵ 7-day differentiated primary macrophages in 6-well plates were pretreated with inhibitors for 4 h then infected with 50 ng HIV-1 BaL (ABI). Cells were infected overnight and fresh complete media with 50 ng/ml of mCSF was added after washing wells with PBS. Nutrient-depleted media was replaced every 3 days with media containing inhibitors. Cells were washed with PBS 10 days post-infection and lysed on plates with 100 ul of RIPA buffer containing protease and phosphatase inhibitors using cell scraper. The data are shown in FIG. 1. The lanes are as follows: (lane 1) DMSO; (lane 2) DMSO+HIV-1 BaL; (lane 3) 10 uM EVJ+HIV-1 BaL; (lane 4) 10 uM JWJ+HIV-1 BaL; (lane 5) 100 nM rapamycin+HIV-1 BaL; and (lane 6) 250 nM torin1+HIV-1 BaL. The data show that PLD inhibitors suppress HIV-1 replication in primary macrophages.

4. Reduction of HIV-1 Gag

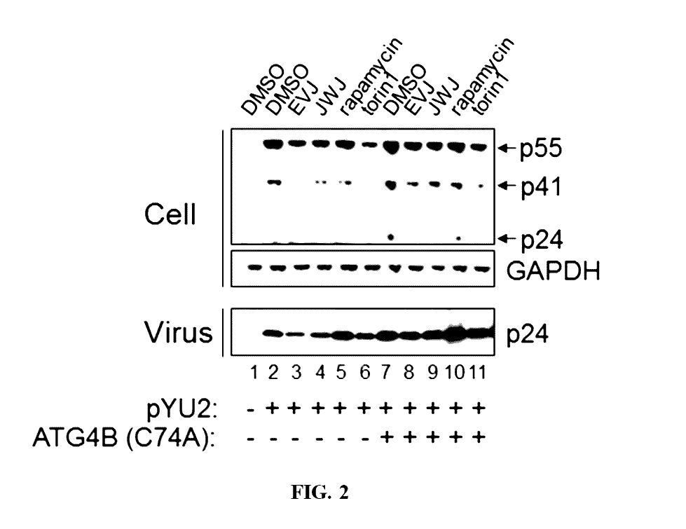

HEK293 cells (0.5×10⁶) were seeded in 6-well plates. The following day cells were transfected with a total of 4 μg DNA (2 μg ATG4B or 2 μg pCDNA3.1 with 2 μg of pYU2) using lipofectamine2000, as suggested by protocol. Four hours after transfection cells were treated with inhibitors (10 μM EVJ, 10 μM JWJ, 100 nM rapamycin, or 250 nM torin1) for a total of 20 hours. Cell culture supernatant was pellet at 100,000×g for 1 h for isolation of virus. Cells were lysed using RIPA buffer and 20 ug of protein was loaded on gel for western blot analysis. Cells lysates were probed for gag expression using a anti-Gag mAb, and anti-GAPDH pAb was used as a loading control. The data are shown in FIG. 2 with treatment indicated above each lane. The data show that dominant-negative ATG4B abrogates PLD inhibitor-dependent reduction of HIV-1 Gag.

5. dNTP Levels

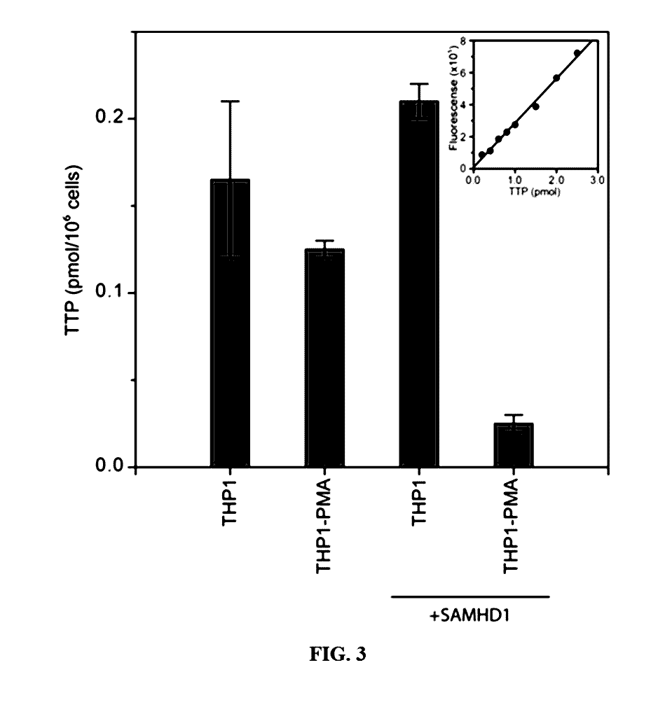

FIG. 3 shows the effect of SamHD1 on dNTP levels in THP-1 cells in the presence and absence of PMA treatment. dNTP levels were determined by HPLC-MS analysis following cell extraction.

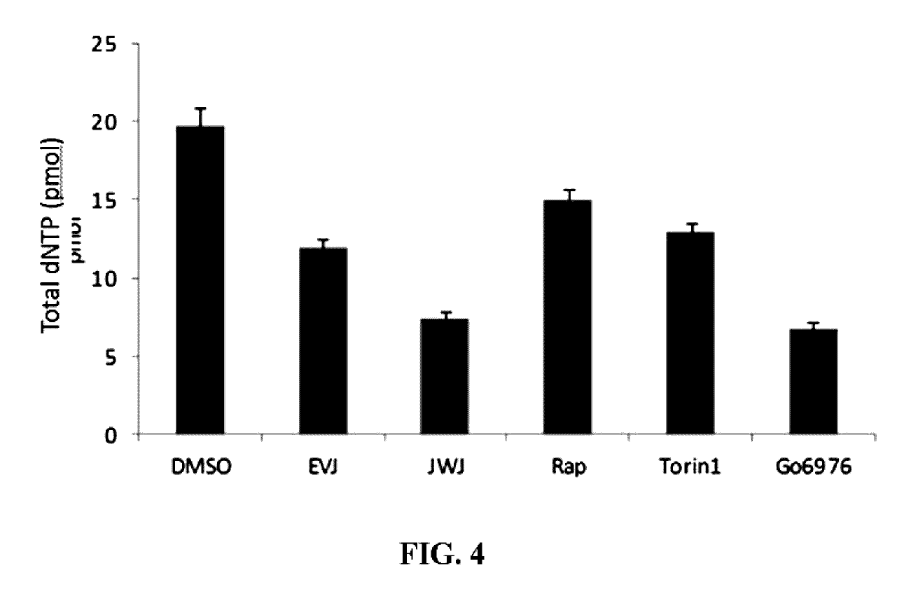

FIG. 4 shows the effect of various treatments on total dNTP levels in THP-1 cells, including the effect of PLD inhibitors. Briefly, Undifferentiated THP-1 cells (5×10̂6 cells/well) were treated with various inhibitors (10 μM EVJ, 10 μM JWJ, 100 nM rapamycin, 250 nM torin1, or 1 μM Go6976) for 16 h before harvest and extraction for HPLC-MS analysis of dNTP levels. Data represents analysis of triplicate samples. The data show that PLD inhibitors reduce dNTP levels in THP-1 cells.

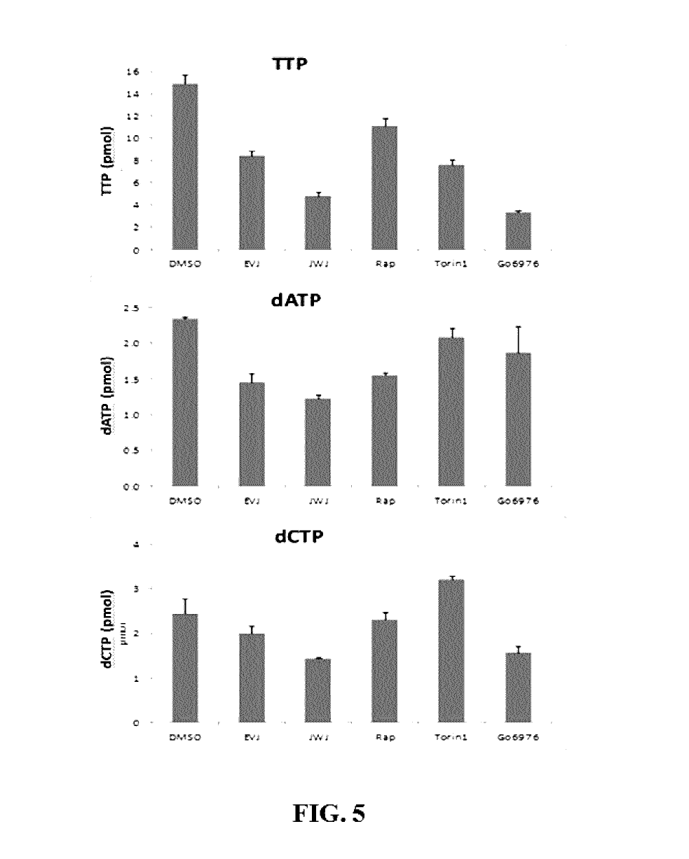

FIG. 5 shows the levels of specific dNTPs in cells following treatment and extraction as described for FIG. 4. As observed in the experiment shown in FIG. 4, each of the dNTPs examined were observed to decrease upon treatment with a representative PLD inhibitor.

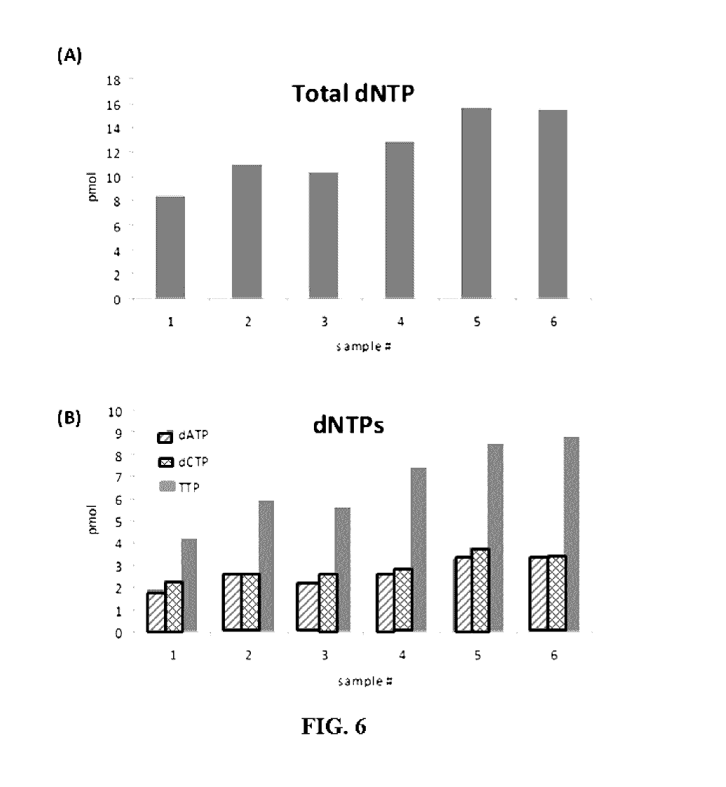

Table 4 below shows the effect of SamHD1 depletion on dNTP levels in THP-1 cells. Briefly, THP-1 cells (2.5×10⁶) were differentiated for 48 h with 1 uM PMA then transfected with 1.25 μg of non-targeting or SamHD1 siRNA using AMAXA (Lonza) program Y-010 and Monocyte transfection reagent as directed by protocol. Cells were harvested and extractions performed for HPLC-MS analysis of dNTPs. It should be noted that samples 1-3 and 4-6 are cells that were transfected with non-targeting and SAMHD1 siRNA, respectively. The data show that SamHD1 depletion increases dNTP levels in THP-1 cells. These data are also shown graphically in FIG. 6.

TABLE 4

| | dATP | dCTP | TTP | Total | std (pk |
|---|---|---|---|---|---|
| # | (pmol) | (pmol) | (pmol) | (pmol) | integration) |
| 1 | 1.9 | 2.3 | 4.2 | 8.4 | 3.4E+06 |
| 2 | 2.5 | 2.6 | 5.9 | 11.0 | 3.2E+06 |
| 3 | 2.1 | 2.6 | 5.6 | 10.3 | 3.1E+06 |
| 4 | 2.6 | 2.8 | 7.4 | 12.8 | 3.1E+06 |
| 5 | 3.3 | 3.8 | 8.5 | 15.6 | 2.8E+06 |
| 6 | 3.3 | 3.4 | 8.5 | 15.5 | 3.1E+06 |
| p-values (1-3 | 0.0392 | 0.0532 | 0.0117 | 0.0173 | |
| vs 4-6) | | | | | |

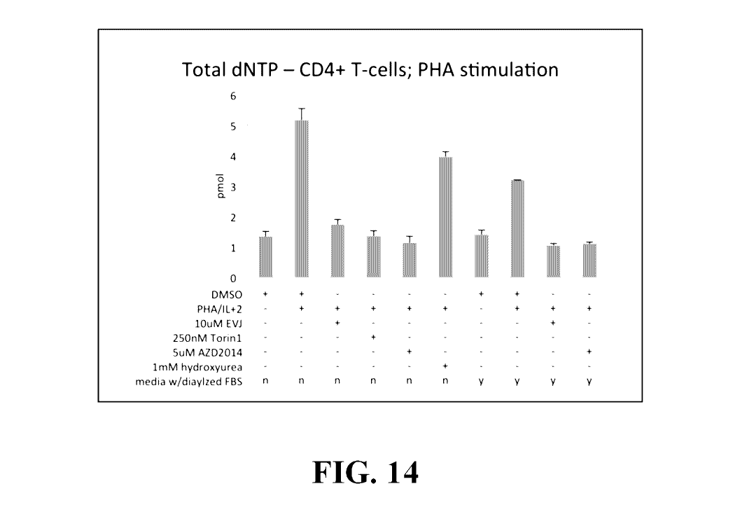

PLD inhibitors modulate intracellular deoxyribonucleotides levels. EVJ and mTOR kinase inhibitors blunt dNTP increases in primary T-cells CD4-receptor stimulated T-cells from human donors (FIG. 14). Specifically, EVJ (10 μM) inhibits HIV-1 replication in activated primary CD4+ T cells (PHA/IL2 activated after EVJ treatment). CD4 T-cells experiments were performed with ani-CD3/CD28-currently completing experiments with IL-2/phytohaemagglutinin (PHA)-stimulated CD4 T cells. Both the PLD inhibitor (EVJ) and mTOR kinase inhibitors (Torin1 and AZD2014) suppress activity of carbamoyl-phosphate synthetase 2, aspartate transcarbamylase, and dihydroorotase (CAD) in IL-2/phytohaemagglutinin (PHA)-stimulated CD4 T cells by reducing phosphorylation at activation residue S1859. Hydroxyurea, an inhibitor of ribonucleotide reductase, has comparative modest effects.

6. Effect of PLD Inhibitors on HIV-1 Infection

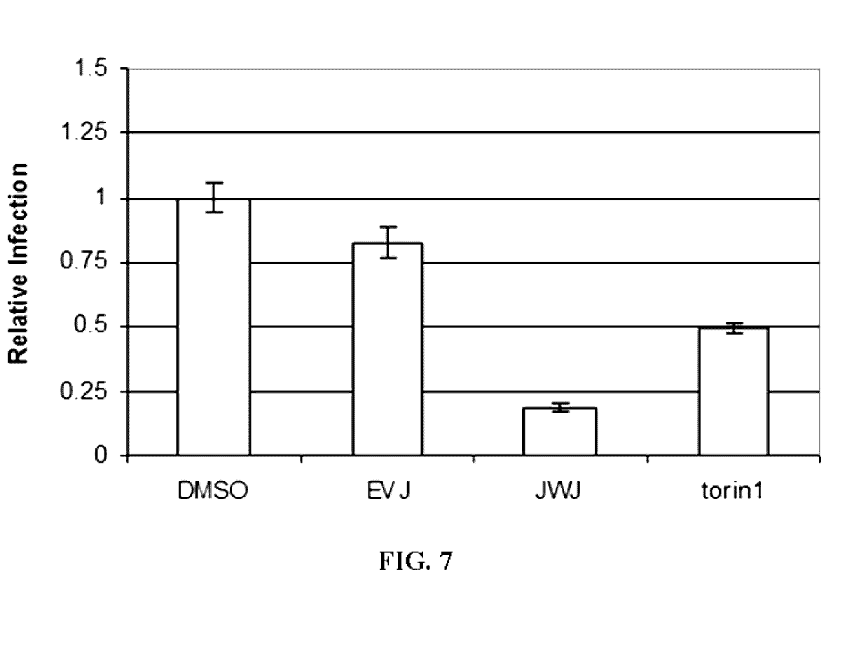

Briefly, THP-1 cells (1×10⁶/mL) were pretreated for 1 h with 10 μM EVJ, 10 μM JWJ, 250 nM torin1 or vehicle (DMSO) then infected with 15 ng of pseudo-typed virus (VSV-G/NLENGL1-GFP (NL4.3-backbone)) and harvested 72 h post-infection. Levels of infection were determined by flow cytometry (BD Biosciences FACSCalibur). The results are shown in FIG. 7. The data show that PLD inhibitors suppress HIV-1 infection in THP-1 cells.

7. HIV-1 Infection in PMA-Stimulated THP-1 Cells

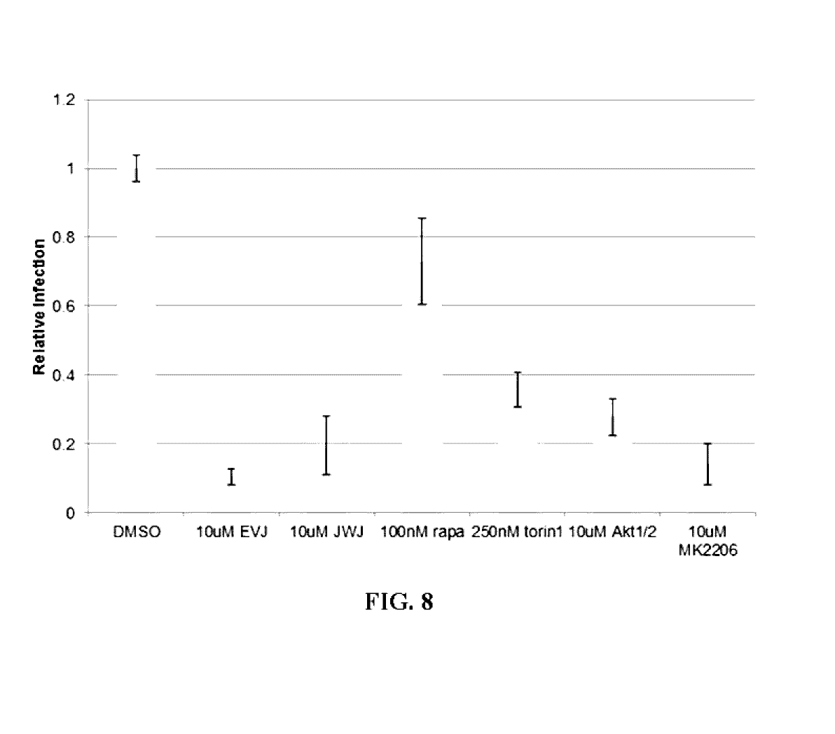

Briefly, THP-1 cells (2.5×10⁵ cells in 0.5 mL of c-RPMI (5×10⁵/mL)/well) were seeded into 24-well plates and differentiated with 0.1 μM PMA for 24 h. Cells were then pretreated for 2 h with inhibitors. NLENG1/VSVg (ing) was added to each well and infection was in the presence of inhibitors for 72 h. Levels of infection were determined by flow cytometry (BD Biosciences FACSCalibur). The results are shown in FIG. 8. The data show that inhibition of PLD or the mTor/Akt pathway reduces HIV-1 infection in PMA-stimulated THP-1 cells.

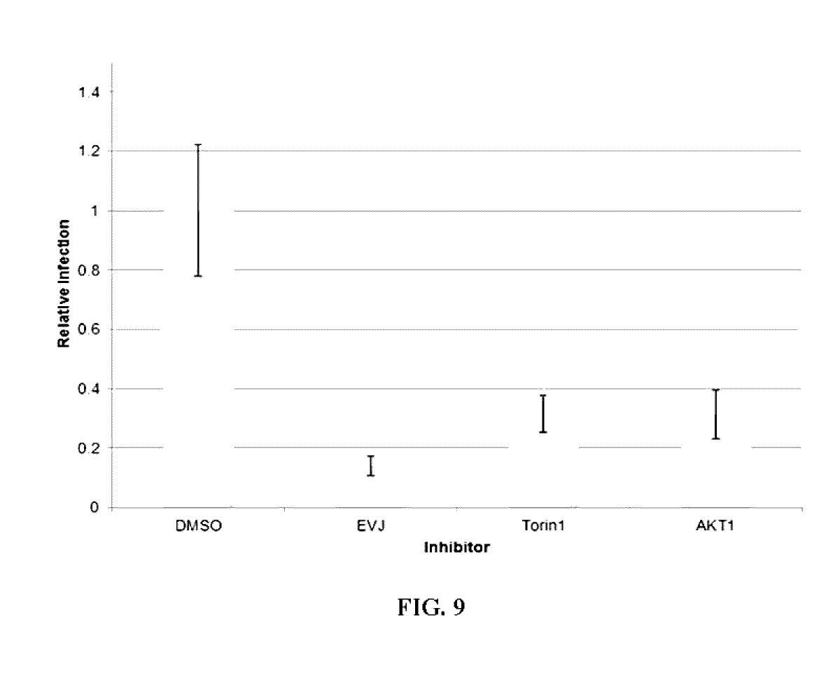

A further experiment is shown in FIG. 9. Briefly, THP-1 cells (2.5×10⁵ cells in 0.5 mL of c-RPMI (5×10⁵/mL)/well) were seeded into 24-well plates and differentiated with 0.1 μM PMA for 24 h. Cells were then pretreated for 12 h with inhibitors (10 μM EVJ, 250 nM torin1, or 10 μM AKT1). NLENG1/VSVg (ing) was added to each well and infection was in the presence of inhibitors for 72 h. Levels of infection were determined by flow cytometry (BD Biosciences FACSCalibur). The data show that PLD, mTor, and Akt inhibitors inhibit HIV-1 replication.

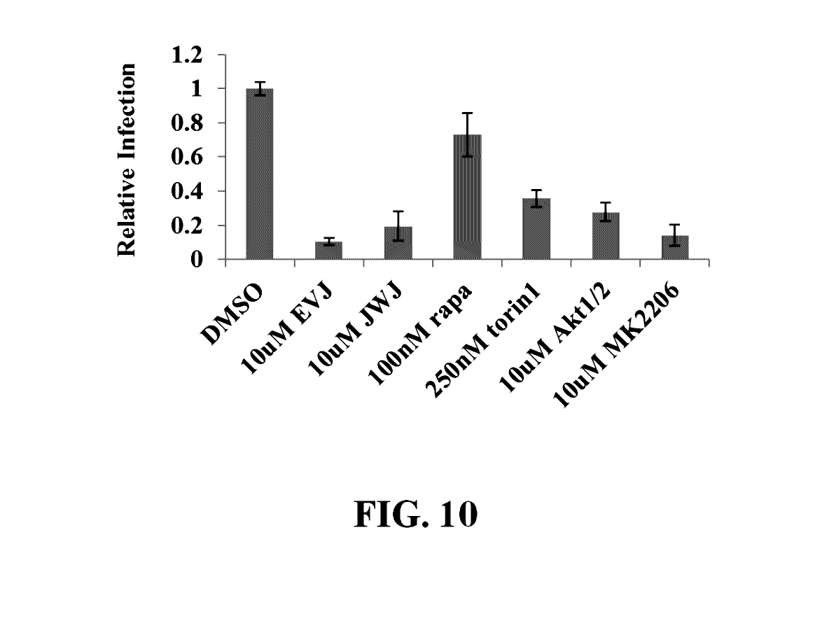

FIG. 10 shows an additional experiment. Briefly, THP-1 cells (2.5×10⁵ cells in 0.5 mL of c-RPMI (5×10⁵/mL)/well) were seeded into 24-well plates and differentiated with 0.1 μM PMA for 24 h. Cells were then pretreated for 4 h with inhibitors (10 μM EVJ, 10 μM JWJ, 100 nM rapamycin, 250 nM torin1, or 10 μM concentration of two AKT inhibitors for comparison). Cells were then infected with HIV-1-GFP in the continued presence of inhibitors. Levels of infection were determined by flow cytometry (BD Biosciences FACSCalibur) 72 h post-infection. The PLD2-preferring inhibitor JWJ inhibits HIV-1 replication under both conditions, whereas the PLD1-preferring inhibitor EVJ has a more robust effect on HIV-1 replication in PMA-differentiated THP-1 cells. This difference is likely due to an increase in PLD1 activity in PMA-treated THP-1 cells. Without wishing to be bound by theory, the data suggest that targeting both isoenzymes systematically is the most rational approach. It is also noteworthy that EVJ and JWJ both inhibit HIV-1 infection of myeloid derived U87.CD4.CXCR4 cells (data not shown). Deoxyribonucleotide (dNTP) levels are potently reduced by both PLD inhibitors, Akt inhibitor MK2206, or mTOR modulator torin1.

8. Role of PLD in Modulation of HIV-1 Infection

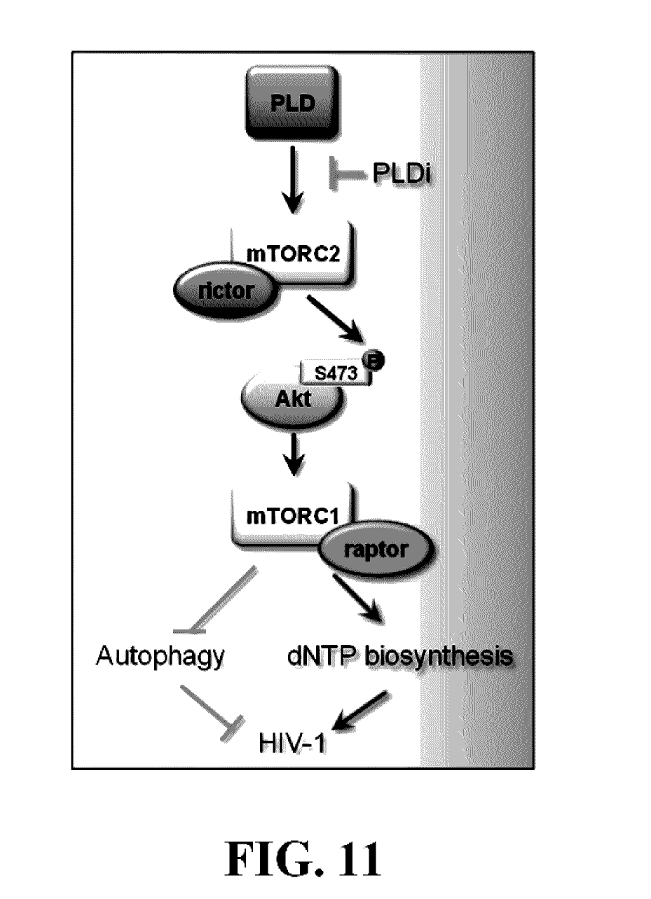

PLD inhibitors suppress mTOR activity in THP-1 cells and primary CD4+ human T-cells (data not shown). Interestingly, mTORC1 activity, assessed by pS6 (S235/236) levels, is suppressed in both cells types. When cells are pretreated with inhibitors and stimulated with agents known to activate mTORC2 (TLR2 ligands or SDF1), both pS6 and pAKT(S473) levels are reduced. Without wishing to be bound by theory, this data suggests that PLD activity falls upstream of mTORC1 activity. mTORC1 controls de novo nucleotide synthesis by controlling pyrimidine biosynthesis via phosphorylation of carbamoyl-phosphate synthetase 2, aspartate transcarbamylase, and dihydroorotase (CAD) and transcriptionally via modification of SREBP-1, which drives expression of many enzymes of the pentose phosphate pathway. It has been demonstrated previously that this pathway falls downstream of AKT activity. Without wishing to be bound by theory, FIG. 11 shows a model that illustrates the proposed role played by PLD in modulation of HIV-1 infection.

9. SILAC Proteomic Analysis of Signaling Pathways Modulated by PLD Inhibitors

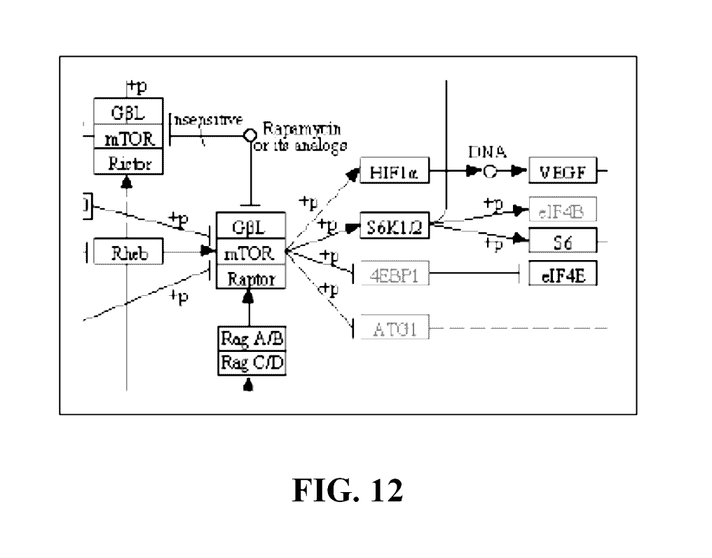

A SILAC proteomic analysis was conducted using the PLD1-preferring inhibitor EVJ on U87MG cells. The data identify signaling pathways responsible for the dNTP phenotype upon EVJ administration. 193 unique protein phosphorylation events changing in the cell were identified using the candidate peptide ID list with the most stringent, common practice statistics (95% confidence interval). Feeding the FASTA IDs into a network-analysis program, mTORC1 signaling is the predominant signaling pathway changed (FIG. 12).

Without wishing to be bound by theory, the prominence of mTORC1 changes in the network analysis further suggests an upstream signaling mechanism responsible for dNTP decreases. A list of noteworthy peptides was assembled (Table 2). Changes in phosphorylation sites on these peptides are intriguing, given their role in the regulation of activity of the proteins to which they belong. These changes are in good agreement with those observed in intracellular metabolite changes as measured by mass spectrometry (i.e., decreases in intracellular dNTP pools). Referring to Table 5, serines (bolded and underlined) indicate the phosphor-site changing according to peptide fragmentation. The change in the phosphorylation site on ser-1859 on CAD that is observed following EVJ treatment is of particular interest. The multifunctional protein CAD (aspartate carbamoyltransferase) enzyme catalyzes the first three steps of pyrimidine do novo synthesis.

TABLE 5

| | | Ratio | | |
|---|---|---|---|---|
| Peptide Sequence | Protein | (H/L) | Phosphosite | Known activation site? |
| GVHIHQAGGSPPASST | RAPTOR | 0.64 | Ser877 & | Ser877 |
| SSSSLTNDVAKQPVSR | | | Ser884 | hyperphosphorylation |
| | | | | increases mTOR activity |
| SLENETLNKEEDCHSP | dIF4B | 0.46 | Ser459 | Annotated activation sites; |
| TSKPPKPDQPLK | | | | upstream kinase unknown |
| DTYSDRSGSSSPDSEIT | CTPS1 | 0.63 | Ser574 & | Annotated activation sites; |
| ELKFPSIN | | | Ser575 | upstream kinase unknown |
| IHRASDPGLPAEEPKE | CAD | 0.76 | Ser1859 | Known activation site of |
| K | | | | catalytic activity |
| KFLMECRNSPVTK | 4EBP1 | 0.40 | Ser65 | Downstream site for |
| | | | | mTORC1 phosphorylation |

10. CAD Overexpression Rescues dNTP Phenotype

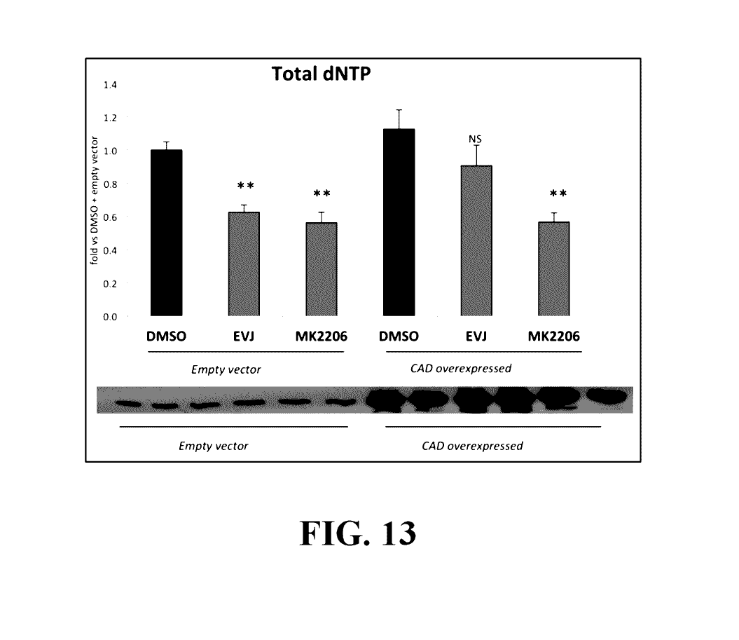

CAD over expression partially rescues the dNTP biosynthetic phenotype observed upon treatment with EVJ (FIG. 13). In contrast, the AKT inhibitor MK2206 continues to block dNTP levels. Without wishing to be bound by theory, these data suggest that there are both AKT-dependent and independent components to PLD-mediated regulation of dNTP levels.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for treating a subject for HIV infection, the method comprising the step of administering to the subject an effective amount of a phospholipase D (PLD) inhibitor, thereby treating the subject for HIV infection.

2. The method of claim 1, wherein the subject is a mammal.

3. The method of claim 1, wherein the subject is a human

4. The method of claim 1, wherein the effective amount is a therapeutically effective amount.

5. The method of claim 1, wherein the effective amount is a prophylactically effective amount.

6. The method of claim 1, wherein the effective amount of a phospholipase D inhibitor inhibits HIV replication.

7. The method of claim 1, wherein the effective amount of a phospholipase D inhibitor inhibits HIV integration.

8. The method of claim 1, wherein the phospholipase D inhibitor inhibits PLD1 and/or PLD2.

9. The method of claim 1, wherein the subject has been diagnosed with a need for treatment of the HIV infection prior to the administering step.

10. The method of claim 1, further comprising the step of identifying a subject in need of treatment of the HIV infection.

11. A pharmaceutical composition comprising an effective amount of a phospholipase D inhibitor, or a pharmaceutically acceptable prodrug, salt, solvate, or polymorph thereof; an effective amount of at least one compound selected from:
a) a HIV fusion/lysis inhibitor, or a pharmaceutically acceptable prodrug, salt, solvate, or polymorph thereof;
b) a HIV integrase inhibitor, or a pharmaceutically acceptable prodrug, salt, solvate, or polymorph thereof;
c) a HIV non-nucleoside reverse transcriptase inhibitor, or a pharmaceutically acceptable prodrug, salt, solvate, or polymorph thereof;
d) a HIV nucleoside reverse transcriptase inhibitor, or a pharmaceutically acceptable prodrug, salt, solvate, or polymorph thereof; and
e) a HIV protease inhibitor, or a pharmaceutically acceptable prodrug, salt, solvate; and
a pharmaceutically acceptable carrier.

12. The composition of claim 11, wherein the effective amount is a therapeutically effective amount.

13. The composition of claim 11, wherein the effective amount is a prophylactically effective amount.

14. The composition of claim 11, wherein the phospholipase D inhibitor inhibits PLD1 and/or PLD2.

15. The composition of claim 11, wherein the composition is formulated for oral administration.

16. The composition of claim 11, wherein the composition is formulated for intravenous administration.

17. A kit comprising a phospholipase D inhibitor, or a pharmaceutically acceptable prodrug, salt, solvate, or polymorph thereof, and one or more of:
a) at least one agent known to treat an HIV infection;
b) at least one agent known to treat an opportunistic infection associated with an HIV infection;
c) instructions for treating an HIV infection;
d) instructions for treating an opportunistic infection associated with an HIV infection;
e) instructions for administering the phospholipase D inhibitor in connection with treating an HIV infection; or
f) instructions for administering the phospholipase D inhibitor in connection with reducing the risk of HIV infection.

18. The kit of claim 17, wherein the phospholipase D inhibitor and the at least one agent are co-packaged.

19. The kit of claim 17, wherein the phospholipase D inhibitor and the at least one agent are co-formulated.

20. The kit of claim 17, further comprising a plurality of dosage forms, the plurality comprising one or more doses; wherein each dose comprises an effective amount of the phospholipase D inhibitor and the at least one agent.
